(19) United States
(12) Patent Application Publication
(10) Pub. No.: US 2024/0426793 A1
(43) Pub. Date: Dec. 26, 2024

(54) METHODS FOR CHROMATOGRAPHY AND CHROMATOGRAPHY MEDIUM REUSE

(71) Applicant: AMGEN INC., Thousand Oaks, CA (US)

(72) Inventors: Nicholas Anthony VECCHIARELLO, Charlottesville, VA (US); Venkatesh NATARAJAN, Arlington, MA (US); Andrew John MALONEY, West Roxbury, MA (US); Benjamin SMITH, Boulder, CO (US); Joseph Edward BASCONI, Cambridge, MA (US)

(73) Assignee: AMGEN INC., Thousand Oaks, CA (US)

(21) Appl. No.: 18/744,883

(22) Filed: Jun. 17, 2024

Related U.S. Application Data
(60) Provisional application No. 63/509,093, filed on Jun. 20, 2023.

(51) Int. Cl. G01N 30/14; G01N 30/02; G01N 30/80; G01N 30/88

(52) U.S. Cl. CPC G01N 30/14; G01N 30/80; G01N 30/88; G01N 2030/027; G01N 2030/8831; G01N 2030/8872

(57) ABSTRACT

Disclosed herein are methods for chromatography, including cleaning methods for chromatography medium that make use of a linear salt gradient. The disclosed methods may be used for frontal chromatography operations and for cleaning frontal chromatography media for reuse in biologics manufacturing.

### CROSS-REFERENCE TO RELATED APPLICATIONS

This application claims the benefit of priority to U.S. Provisional Patent Application No. 63/509,093, filed Jun. 20, 2023, which is hereby incorporated by reference in its entirety.

### FIELD

The present disclosure provides methods for chromatography, including cleaning methods for chromatography medium that make use of a linear salt gradient.

### BACKGROUND

In biologics drug substance manufacturing, high productivity upstream and downstream operations (i.e., operations capable of processing a high mass of drug substance) are critical for high-volume commercial manufacturing. Drug substance manufacturing operations must consistently provide high-quality products that meet or exceed attribute requirements for safety, potency, purity, and efficacy, at volumes sufficient to meet patient demand. In addition, drug substance manufacturing operations should be sustainable, robust, cost effective, and compliant with regulatory requirements, all while maintaining efficient processing within the footprint and time constraints of the manufacturing facility.

Upstream operations comprise culturing cells engineered to express a product of interest and harvesting the recombinant product. Downstream operations purify the recombinant product away from product- and process-related impurities and contaminants to meet product quality and yield requirements for the final bulk drug substance. Downstream operations from capture to polishing make use of a variety of preparative chromatography methods.

Contaminants and process- and product-related impurities are removed to varying degrees by different types of chromatography media, and downstream purification processes commonly use multiple types of chromatography media to meet product quality requirements. Capture operations, such as affinity chromatography, may remove process-related impurities and contaminants, while polishing chromatography operations can be employed to remove product-related impurities and contaminants, such as high molecular weight product-related species. Advances in chromatography technology have enabled increasing loading densities; accordingly, chromatography media may experience high levels of impurity saturation at the end of a cycle or batch.

Chromatography medium cleaning is an integral part of clinical and commercial manufacturing processes that affects product safety, chromatography medium longevity, and process robustness. Chromatography medium cleaning may mitigate potential carryover of impurities and contaminants from one cycle to another, as well as fouling, bioburden, and degradation of the resin within a packed column, thus enabling an increased number of lifetime cycles (i.e., the number of times that the chromatography medium may be recycled without significant degradation of performance that affects the yield, purity, or other attributes of the purified product).

Advances in cell line development as well as culture and harvest methods have led to higher titer product pools for downstream purification. Increased product titers, culture volumes, culture cell densities, and impurity levels associated with intensified upstream operations present challenges for downstream operations, including preparative chromatography operations. To enable efficient downstream purification, there remains a need in the art for additional chromatography operations that can achieve high loading densities and robustly separate impurities and contaminants from the product of interest over many cycles.

### SUMMARY

Some embodiments of the present disclosure relate to a method of cleaning a chromatography medium for reuse, comprising:
loading a composition comprising a protein and at least one impurity onto the chromatography medium;
collecting fractions comprising the protein; and
cleaning the chromatography medium using a linear salt gradient.

In some embodiments, the loading density exceeds the dynamic binding capacity of the chromatography medium for the protein. In some embodiments, the loading density is at least 200 g/L-r. In some embodiments, the loading density is at least 1000 g/L-r. In some embodiments, the loading density is in the range of 1000 g/L-r to 1500 g/L-r.

In some embodiments, the chromatography medium is used in frontal mode for the purification of the protein.

In some embodiments, the chromatography medium is loaded at or near saturation with the at least one impurity. In some embodiments, the at least one impurity comprises one or more high molecular weight species of the protein.

In some embodiments, the linear salt gradient comprises an increase in salt concentration from less than 50 mM to 500 mM. In some embodiments, the linear salt gradient comprises an increase in salt concentration from 0 mM or 20 mM to 500 mM.

In some embodiments, the linear salt gradient is an NaCl gradient, a KCl gradient, a CaCl₂ gradient, or an Na₂SO₄ gradient. In some embodiments, the linear salt gradient is an NaCl gradient.

In some embodiments, the linear salt gradient is generated using at least two buffers. In some embodiments, each of the at least two buffers used to generate the linear salt gradient is independently selected from acetate buffers, phosphate buffers, Tris buffers, and 2-(N-morpholino) ethanesulfonic acid buffers. In some embodiments, each of the at least two buffers used to generate the linear salt gradient comprises acetate. In some embodiments, each of the at least two buffers used to generate the linear salt gradient comprises acetate at a concentration of 50 mM. In some embodiments, the pH of each of the at least two buffers used to generate the linear salt gradient is greater than 3.6. In some embodiments, the pH of each of the at least two buffers used to generate the linear salt gradient is less than 5.6.

In some embodiments, the linear salt gradient is generated by Buffer A and Buffer B, wherein Buffer A comprises 50 mM acetate and 0 mM or 20 mM sodium chloride at a pH value of 5.0±0.1 and Buffer B comprises 50 mM acetate and 500 mM sodium chloride at a pH value of 5.0±0.1. In some embodiments, the linear salt gradient is generated using Buffer A and Buffer B in a gradient from 100% Buffer A (0% Buffer B) to 0% Buffer A (100% Buffer B), or 90% Buffer A (10% Buffer B) to 10% Buffer A (90% Buffer B), or 80% Buffer A (20% Buffer B) to 20% Buffer A (80% Buffer B).

In some embodiments, the slope of the linear salt gradient is less than or equal to 0.07 M salt/medium volume buffer.

In some embodiments, the gradient length is at least 7 medium volumes.

In some embodiments, the cleaning using the linear salt gradient is non-denaturing.

In some embodiments, the cleaning using the linear salt gradient is performed after at least one cycle. In some embodiments, the cleaning using the linear salt gradient is performed after at least one batch. In some embodiments, the cleaning using the linear salt gradient is performed prior to storage.

In some embodiments, the method further comprises cleaning the chromatography medium using a denaturing solution. In some embodiments, the denaturing solution comprises sodium hydroxide. In some embodiments, the denaturing solution comprises sodium hydroxide at a concentration of 1 M. In some embodiments, 3 medium volumes of the denaturing solution are passed through the chromatography medium.

In some embodiments, the cleaning using the denaturing solution is performed after at least one cycle. In some embodiments, the cleaning using the denaturing solution is performed after at least one batch. In some embodiments, the cleaning using the denaturing solution is performed prior to storage.

In some embodiments, the denaturing solution is used in an isocratic cleaning process.

In some embodiments, the chromatography medium is stored in a storage solution comprising sodium hydroxide at a concentration in the range of 0.1 M to 0.2 M. In some embodiments, the chromatography medium is stored in a storage solution comprising sodium hydroxide at a concentration of 0.1 M. In some embodiments, the chromatography medium is stored in a storage solution comprising sodium hydroxide at a concentration of 0.2 M.

In some embodiments, the chromatography medium is a cation exchange chromatography medium. In some embodiments, the chromatography medium is a cation exchange chromatography resin. In some embodiments, the chromatography medium is a cation exchange chromatography resin comprising tentacle ligands (e.g., Eshmuno® CP-FT resin).

In some embodiments, the chromatography medium is packed in a chromatography column.

In some embodiments, the chromatography medium is a cation exchange chromatography medium and is packed in a chromatography column. In some embodiments, the chromatography medium is a cation exchange chromatography resin and is packed in a chromatography column. In some embodiments, the chromatography medium is a cation exchange chromatography resin comprising tentacle ligands (e.g., Eshmuno® CP-FT resin) and is packed in a chromatography column.

In some embodiments, the method enables reuse of the chromatography medium for multiple (e.g., at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100; 5, 10, 15, 20, 25, 30, 35, 40, 45, 50, 55, 60, 65, 70, 75, 80, 85, 90, 95, 100) cycles. In some embodiments, the method enables reuse of the chromatography medium for at least 10 cycles. In some embodiments, the method enables reuse of the chromatography medium for at least 20 cycles. In some embodiments, the method enables reuse of the chromatography medium for at least 30 cycles. In some embodiments, the method enables reuse of the chromatography medium for at least 40 cycles. In some embodiments, the method enables reuse of the chromatography medium for at least 50 cycles. In some embodiments, the method enables reuse of the chromatography medium for at least 60 cycles. In some embodiments, the method enables reuse of the chromatography medium for at least 70 cycles. In some embodiments, the method enables reuse of the chromatography medium for at least 80 cycles. In some embodiments, the method enables reuse of the chromatography medium for at least 90 cycles. In some embodiments, the method enables reuse of the chromatography medium for at least 100 cycles.

In some embodiments, the linear velocity during the loading is in the range of 100 cm/hr to 250 cm/hr. In some embodiments, the linear velocity during the loading is in the range of 100 cm/hr to 200 cm/hr. In some embodiments, the linear velocity during the loading is in the range of 150 cm/hr to 250 cm/hr. In some embodiments, the linear velocity during the loading is in the range of 150 cm/hr to 200 cm/hr.

In some embodiments, the linear velocity during the loading is 150 cm/hr. In some embodiments, the linear velocity during the loading is 200 cm/hr.

In some embodiments, the linear velocity during the cleaning is in the range of 100 cm/hr to 250 cm/hr. In some embodiments, the linear velocity during the cleaning is in the range of 100 cm/hr to 200 cm/hr. In some embodiments, the linear velocity during the cleaning is in the range of 125 cm/hr to 250 cm/hr. In some embodiments, the linear velocity during the cleaning is in the range of 125 cm/hr to 200 cm/hr.

In some embodiments, the linear velocity during the cleaning is 133 cm/hr. In some embodiments, the linear velocity during the cleaning is 200 cm/hr.

Some embodiments of the present disclosure relate to a method of cleaning a chromatography medium for reuse, comprising:
loading a composition comprising a protein and at least one impurity onto the chromatography medium, wherein the loading density exceeds the dynamic binding capacity of the chromatography medium for the protein;
collecting fractions comprising the protein; and
cleaning the chromatography medium using a linear salt gradient, wherein:
   the linear salt gradient is an NaCl gradient, a KCl gradient, a CaCl₂ gradient, or an Na₂SO₄ gradient; and
   the linear salt gradient comprises an increase in salt concentration from less than 50 mM to 500 mM.

In some embodiments, the loading density is in the range of 1000 g/L-r to 1500 g/L-r.

In some embodiments, the chromatography medium is a cation exchange chromatography medium. In some embodiments, the chromatography medium is a cation exchange chromatography resin. In some embodiments, the chromatography medium is a cation exchange chromatography resin comprising tentacle ligands (e.g., Eshmuno® CP-FT resin).

In some embodiments, the chromatography medium is packed in a chromatography column.

In some embodiments, the chromatography medium is a cation exchange chromatography medium and is packed in a chromatography column. In some embodiments, the chromatography medium is a cation exchange chromatography resin and is packed in a chromatography column. In some embodiments, the chromatography medium is a cation exchange chromatography resin comprising tentacle ligands (e.g., Eshmuno® CP-FT resin) and is packed in a chromatography column.

In some embodiments, the linear salt gradient is an NaCl gradient.

In some embodiments, the slope of the linear salt gradient is less than or equal to 0.07 M salt/medium volume buffer. In some embodiments, the gradient length is at least 7 medium volumes. In some embodiments, the slope of the linear salt gradient is less than or equal to 0.07 M salt/medium volume buffer, and the gradient length is at least 7 medium volumes.

In some embodiments, the linear salt gradient is an NaCl gradient, the slope of the linear salt gradient is less than or equal to 0.07 M salt/medium volume buffer, and the gradient length is at least 7 medium volumes.

In some embodiments, the method enables reuse of the chromatography medium for multiple (e.g., at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100; 5, 10, 15, 20, 25, 30, 35, 40, 45, 50, 55, 60, 65, 70, 75, 80, 85, 90, 95, 100) cycles. In some embodiments, the method enables reuse of the chromatography medium for at least 10 cycles. In some embodiments, the method enables reuse of the chromatography medium for at least 20 cycles. In some embodiments, the method enables reuse of the chromatography medium for at least 30 cycles. In some embodiments, the method enables reuse of the chromatography medium for at least 40 cycles. In some embodiments, the method enables reuse of the chromatography medium for at least 50 cycles. In some embodiments, the method enables reuse of the chromatography medium for at least 60 cycles. In some embodiments, the method enables reuse of the chromatography medium for at least 70 cycles. In some embodiments, the method enables reuse of the chromatography medium for at least 80 cycles. In some embodiments, the method enables reuse of the chromatography medium for at least 90 cycles. In some embodiments, the method enables reuse of the chromatography medium for at least 100 cycles.

In some embodiments, the linear velocity during the loading is in the range of 100 cm/hr to 250 cm/hr. In some embodiments, the linear velocity during the loading is in the range of 100 cm/hr to 200 cm/hr. In some embodiments, the linear velocity during the loading is in the range of 150 cm/hr to 250 cm/hr. In some embodiments, the linear velocity during the loading is in the range of 150 cm/hr to 200 cm/hr.

In some embodiments, the linear velocity during the loading is 150 cm/hr. In some embodiments, the linear velocity during the loading is 200 cm/hr.

In some embodiments, the linear velocity during the cleaning is in the range of 100 cm/hr to 250 cm/hr. In some embodiments, the linear velocity during the cleaning is in the range of 100 cm/hr to 200 cm/hr. In some embodiments, the linear velocity during the cleaning is in the range of 125 cm/hr to 250 cm/hr. In some embodiments, the linear velocity during the cleaning is in the range of 125 cm/hr to 200 cm/hr.

In some embodiments, the linear velocity during the cleaning is 133 cm/hr. In some embodiments, the linear velocity during the cleaning is 200 cm/hr.

Some embodiments of the present disclosure relate to a method of cleaning a chromatography medium for reuse, comprising:
loading a composition comprising a protein and at least one impurity onto the chromatography medium, wherein the loading density exceeds the dynamic binding capacity of the chromatography medium for the protein;
collecting fractions comprising the protein; and
cleaning the chromatography medium using a linear salt gradient, wherein the linear salt gradient is generated by Buffer A and Buffer B, wherein Buffer A comprises 50 mM acetate and 0 mM or 20 mM sodium chloride at a pH value of 5.0±0.1 and Buffer B comprises 50 mM acetate and 500 mM sodium chloride at a pH value of 5.0±0.1.

In some embodiments, the loading density is in the range of 1000 g/L-r to 1500 g/L-r.

In some embodiments, the chromatography medium is a cation exchange chromatography medium. In some embodiments, the chromatography medium is a cation exchange chromatography resin. In some embodiments, the chromatography medium is a cation exchange chromatography resin comprising tentacle ligands (e.g., Eshmuno® CP-FT resin).

In some embodiments, the chromatography medium is packed in a chromatography column.

In some embodiments, the chromatography medium is a cation exchange chromatography medium and is packed in a chromatography column. In some embodiments, the chromatography medium is a cation exchange chromatography resin and is packed in a chromatography column. In some embodiments, the chromatography medium is a cation exchange chromatography resin comprising tentacle ligands (e.g., Eshmuno® CP-FT resin) and is packed in a chromatography column.

In some embodiments, the linear salt gradient is generated using Buffer A and Buffer B in a gradient from 100% Buffer A (0% Buffer B) to 0% Buffer A (100% Buffer B), or 90% Buffer A (10% Buffer B) to 10% Buffer A (90% Buffer B), or 80% Buffer A (20% Buffer B) to 20% Buffer A (80% Buffer B).

In some embodiments, the slope of the linear salt gradient is less than or equal to 0.07 M salt/medium volume buffer. In some embodiments, the gradient length is at least 7 medium volumes. In some embodiments, the slope of the linear salt gradient is less than or equal to 0.07 M salt/medium volume buffer, and the gradient length is at least 7 medium volumes.

In some embodiments, the method enables reuse of the chromatography medium for multiple (e.g., at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100; 5, 10, 15, 20, 25, 30, 35, 40, 45, 50, 55, 60, 65, 70, 75, 80, 85, 90, 95, 100) cycles. In some embodiments, the method enables reuse of the chromatography medium for at least 10 cycles. In some embodiments, the method enables reuse of the chromatography medium for at least 20 cycles. In some embodiments, the method enables reuse of the chromatography medium for at least 30 cycles. In some embodiments, the method enables reuse of the chromatography medium for at least 40 cycles. In some embodiments, the method enables reuse of the chromatography medium for at least 50 cycles. In some embodiments, the method enables reuse of the chromatography medium for at least 60 cycles. In some embodiments, the method enables reuse of the chromatography medium for at least 70 cycles. In some embodiments, the method enables reuse of the chromatography medium for at least 80 cycles. In some embodiments, the method enables reuse of the chromatography medium for at least 90 cycles. In some embodiments, the method enables reuse of the chromatography medium for at least 100 cycles.

In some embodiments, the linear velocity during the loading is in the range of 100 cm/hr to 250 cm/hr. In some embodiments, the linear velocity during the loading is in the range of 100 cm/hr to 200 cm/hr. In some embodiments, the linear velocity during the loading is in the range of 150 cm/hr to 250 cm/hr. In some embodiments, the linear velocity during the loading is in the range of 150 cm/hr to 200 cm/hr.

In some embodiments, the linear velocity during the loading is 150 cm/hr. In some embodiments, the linear velocity during the loading is 200 cm/hr.

In some embodiments, the linear velocity during the cleaning is in the range of 100 cm/hr to 250 cm/hr. In some embodiments, the linear velocity during the cleaning is in the range of 100 cm/hr to 200 cm/hr. In some embodiments, the linear velocity during the cleaning is in the range of 125 cm/hr to 250 cm/hr. In some embodiments, the linear velocity during the cleaning is in the range of 125 cm/hr to 200 cm/hr.

In some embodiments, the linear velocity during the cleaning is 133 cm/hr. In some embodiments, the linear velocity during the cleaning is 200 cm/hr.

Some embodiments of the present disclosure relate to a method of cleaning a chromatography medium for reuse, comprising:
loading a composition comprising a protein and at least one impurity onto the chromatography medium, wherein the loading density exceeds the dynamic binding capacity of the chromatography medium for the protein;
collecting fractions comprising the protein; and
cleaning the chromatography medium using a linear salt gradient, wherein the slope of the linear salt gradient is less than or equal to 0.07 M salt/medium volume buffer.

In some embodiments, the loading density is in the range of 1000 g/L-r to 1500 g/L-r.

In some embodiments, the chromatography medium is a cation exchange chromatography medium. In some embodiments, the chromatography medium is a cation exchange chromatography resin. In some embodiments, the chromatography medium is a cation exchange chromatography resin comprising tentacle ligands (e.g., Eshmuno® CP-FT resin).

In some embodiments, the chromatography medium is packed in a chromatography column.

In some embodiments, the chromatography medium is a cation exchange chromatography medium and is packed in a chromatography column. In some embodiments, the chromatography medium is a cation exchange chromatography resin and is packed in a chromatography column. In some embodiments, the chromatography medium is a cation exchange chromatography resin comprising tentacle ligands (e.g., Eshmuno® CP-FT resin) and is packed in a chromatography column.

In some embodiments, the linear salt gradient is an NaCl gradient, a KCl gradient, a CaCl₂ gradient, or an Na₂SO₄ gradient. In some embodiments, the linear salt gradient is an NaCl gradient.

In some embodiments, the gradient length is at least 7 medium volumes.

In some embodiments, the linear salt gradient is an NaCl gradient, and the gradient length is at least 7 medium volumes.

In some embodiments, the method enables reuse of the chromatography medium for multiple (e.g., at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100; 5, 10, 15, 20, 25, 30, 35, 40, 45, 50, 55, 60, 65, 70, 75, 80, 85, 90, 95, 100) cycles. In some embodiments, the method enables reuse of the chromatography medium for at least 10 cycles. In some embodiments, the method enables reuse of the chromatography medium for at least 20 cycles. In some embodiments, the method enables reuse of the chromatography medium for at least 30 cycles. In some embodiments, the method enables reuse of the chromatography medium for at least 40 cycles. In some embodiments, the method enables reuse of the chromatography medium for at least 50 cycles. In some embodiments, the method enables reuse of the chromatography medium for at least 60 cycles. In some embodiments, the method enables reuse of the chromatography medium for at least 70 cycles. In some embodiments, the method enables reuse of the chromatography medium for at least 80 cycles. In some embodiments, the method enables reuse of the chromatography medium for at least 90 cycles. In some embodiments, the method enables reuse of the chromatography medium for at least 100 cycles.

In some embodiments, the linear velocity during the loading is in the range of 100 cm/hr to 250 cm/hr. In some embodiments, the linear velocity during the loading is in the range of 100 cm/hr to 200 cm/hr. In some embodiments, the linear velocity during the loading is in the range of 150 cm/hr to 250 cm/hr. In some embodiments, the linear velocity during the loading is in the range of 150 cm/hr to 200 cm/hr.

In some embodiments, the linear velocity during the loading is 150 cm/hr. In some embodiments, the linear velocity during the loading is 200 cm/hr.

In some embodiments, the linear velocity during the cleaning is in the range of 100 cm/hr to 250 cm/hr. In some embodiments, the linear velocity during the cleaning is in the range of 100 cm/hr to 200 cm/hr. In some embodiments, the linear velocity during the cleaning is in the range of 125 cm/hr to 250 cm/hr. In some embodiments, the linear velocity during the cleaning is in the range of 125 cm/hr to 200 cm/hr.

In some embodiments, the linear velocity during the cleaning is 133 cm/hr. In some embodiments, the linear velocity during the cleaning is 200 cm/hr.

Some embodiments of the present disclosure relate to a method for controlling operating pressure during cleaning of a chromatography medium for reuse, comprising:
cleaning the chromatography medium using a linear salt gradient; and
cleaning the chromatography medium using a denaturing solution.

In some embodiments, the denaturing solution is used in an isocratic cleaning process.

In some embodiments, the chromatography medium is a cation exchange chromatography medium. In some embodiments, the chromatography medium is a cation exchange chromatography resin. In some embodiments, the chromatography medium is a cation exchange chromatography resin comprising tentacle ligands (e.g., Eshmuno® CP-FT resin).

In some embodiments, the chromatography medium is packed in a chromatography column.

In some embodiments, the chromatography medium is a cation exchange chromatography medium and is packed in a chromatography column. In some embodiments, the chromatography medium is a cation exchange chromatography resin and is packed in a chromatography column. In some embodiments, the chromatography medium is a cation exchange chromatography resin comprising tentacle ligands (e.g., Eshmuno® CP-FT resin) and is packed in a chromatography column.

In some embodiments, the method enables reuse of the chromatography medium for multiple (e.g., at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100; 5, 10, 15, 20, 25, 30, 35, 40, 45, 50, 55, 60, 65, 70, 75, 80, 85, 90, 95, 100) cycles. In some embodiments, the method enables reuse of the chromatography medium for at least 10 cycles. In some embodiments, the method enables reuse of the chromatography medium for at least 20 cycles. In some embodiments, the method enables reuse of the chromatography medium for at least 30 cycles. In some embodiments, the method enables reuse of the chromatography medium for at least 40 cycles. In some embodiments, the method enables reuse of the chromatography medium for at least 50 cycles. In some embodiments, the method enables reuse of the chromatography medium for at least 60 cycles. In some embodiments, the method enables reuse of the chromatography medium for at least 70 cycles. In some embodiments, the method enables reuse of the chromatography medium for at least 80 cycles. In some embodiments, the method enables reuse of the chromatography medium for at least 90 cycles. In some embodiments, the method enables reuse of the chromatography medium for at least 100 cycles.

Some embodiments of the present disclosure relate to a method for purifying a protein from a composition comprising the protein and at least one impurity, comprising
equilibrating a chromatography medium using an equilibration solution;
loading the composition comprising the protein and the at least one impurity onto the chromatography medium in frontal mode at a loading density of at least 500 g/L-r;
collecting fractions comprising the protein;
passing less than two medium volumes of a post-load wash solution through the chromatography medium and adding the eluate to the fractions;
cleaning the chromatography medium using a linear salt gradient; and
cleaning the chromatography medium using a denaturing solution.

In some embodiments, the loading density is in the range of 1000 g/L-r to 1500 g/L-r.

In some embodiments, fraction collection begins at 0.5 optical density (OD), A280 (absorbance at 280 nm). In some embodiments, fraction collection is stopped after less than or equal to 1 medium volume of the post-load wash solution is passed through the chromatography medium.

In some embodiments, the equilibration solution does not contain salt.

In some embodiments, the denaturing solution is used in an isocratic cleaning process.

In some embodiments, the chromatography medium is a cation exchange chromatography medium. In some embodiments, the chromatography medium is a cation exchange chromatography resin.

In some embodiments, the chromatography medium is packed in a chromatography column.

In some embodiments, the chromatography medium is a cation exchange chromatography medium and is packed in a chromatography column. In some embodiments, the chromatography medium is a cation exchange chromatography resin and is packed in a chromatography column.

Some embodiments of the present disclosure relate to a method for purifying a protein from a composition comprising the protein and at least one impurity, comprising:
initiating a cell culture in a bioreactor;
culturing cells engineered to express the protein;
harvesting cell culture broth comprising the protein;
loading the harvested cell culture broth onto an affinity chromatography medium in bind and elute mode;
collecting affinity chromatography elution fractions comprising the protein;
loading the affinity chromatography elution fractions onto a cation exchange (CEX) chromatography medium at a loading density of at least 500 g/L-r in frontal mode;
collecting CEX chromatography fractions comprising the protein;
cleaning the CEX chromatography medium using a linear salt gradient; and
cleaning the CEX chromatography medium using a denaturing solution.

In some embodiments, the affinity chromatography medium is a Protein A affinity chromatography medium.

In some embodiments, the affinity chromatography elution fractions are subjected to low pH viral inactivation. In some embodiments, the affinity chromatography elution fractions are subjected to low pH viral inactivation and neutralization post-viral inactivation. In some embodiments, the neutralization pH is at least 5.0 (such as, e.g., 5.0). In some embodiments, the protein is eluted from the affinity chromatography medium at a pH suitable for viral inactivation. In some embodiments, the protein is eluted from the affinity chromatography medium at a pH of less than or equal to 4.0. In some embodiments, the pH of the eluted protein is adjusted to a pH of at least 5.0 (such as, e.g., 5.0).

In some embodiments, the affinity chromatography elution fractions are loaded onto the CEX chromatography medium in a loading time sufficient to achieve viral inactivation. In some embodiments, the loading time is at least 30 minutes.

In some embodiments, the chromatography medium is a cation exchange chromatography resin. In some embodiments, the chromatography medium is a cation exchange chromatography resin comprising tentacle ligands (e.g., Eshmuno® CP-FT resin). In some embodiments, the CEX chromatography medium is packed in a chromatography column. In some embodiments, the chromatography medium is a cation exchange chromatography resin and is packed in a chromatography column. In some embodiments, the chromatography medium is a cation exchange chromatography resin comprising tentacle ligands (e.g., Eshmuno® CP-FT resin) and is packed in a chromatography column.

Some embodiments of the present disclosure relate to a method for improving step yield for a frontal chromatography operation, the method comprising: estimating an amount of a protein in a load solution; and determining a bed height for a frontal chromatography medium based on a target residence time, wherein the flow rate is a constant value in the range of 50 cm/hr to 250 cm/hr (such as, e.g., 50 cm/hr, 75 cm/hr, 100 cm/hr, 125 cm/hr, 150 cm/hr, 175 cm/hr, 200 cm/hr, 225 cm/hr, 250 cm/hr).

In some embodiments, the flow rate is in the range of 100 cm/hr to 250 cm/r. In some embodiments, the flow rate is in the range of 100 cm/hr to 200 cm/hr.

In some embodiments, the flow rate is 133 cm/hr or 200 cm/hr. In some embodiments, the flow rate is 133 cm/hr. In some embodiments, the flow rate is 200 cm/hr.

In some embodiments, a bed diameter for the frontal chromatography medium is held constant.

In some embodiments, the bed diameter is greater than 1 cm, greater than 2 cm, greater than 3 cm, greater than 4 cm, greater than 5 cm, greater than 10 cm, greater than 15 cm, greater than 20 cm, greater than 25 cm, greater than 30 cm, greater than 35 cm, greater than 40 cm, or greater than 45 cm. In some embodiments of methods described herein, the bed diameter is greater than 80 cm, greater than 100 cm, or greater than 120 cm.

In some embodiments, the bed diameter is greater than 1 cm. In some embodiments, the bed diameter is greater than 10 cm.

In some embodiments, the bed diameter is 1 cm, 2 cm, 3 cm, 4 cm, 5 cm, 10 cm, 15 cm, 20 cm, 25 cm, 30 cm, 35 cm, 40 cm, or 45 cm. In some embodiments, the bed diameter is 50 cm, 60 cm, 70 cm, 80 cm, 90 cm, 100 cm, 110 cm, 120 cm, 130 cm, 140 cm, 150 cm, 160 cm, 170 cm, 180 cm, 190, or 200 cm.

In some embodiments, the bed diameter is in the range of 1 cm to 20 cm. In some embodiments, the bed diameter is 1 cm, 10 cm, or 20 cm. In some embodiments, the bed diameter is 1 cm. In some embodiments, the bed diameter is 10 cm. In some embodiments, the bed diameter is 20 cm.

In some embodiments, the bed height is a constant value in the range of 5 cm to 30 cm. In some embodiments, the bed height is a constant value in the range of 10 cm to 20 cm.

In some embodiments, the amount of protein in the load solution is estimated based on monitoring performed in real-time, near real-time, and/or offline. In some embodiments, the amount of protein in the load solution is estimated based on historical data. In some embodiments, the estimate is performed after one or more cycles. In some embodiments, the estimate is performed after one or more batches.

In some embodiments, the frontal chromatography medium is a cation exchange chromatography resin. In some embodiments, the frontal chromatography medium is a cation exchange chromatography resin comprising tentacle ligands (e.g., Eshmuno® CP-FT resin). In some embodiments, the frontal chromatography medium is packed in a chromatography column. In some embodiments, the frontal chromatography medium is a cation exchange chromatography resin and is packed in a chromatography column. In some embodiments, the frontal chromatography medium is a cation exchange chromatography resin comprising tentacle ligands (e.g., Eshmuno® CP-FT resin) and is packed in a chromatography column.

Some embodiments of the present disclosure relate to a method for improving step yield for a frontal chromatography operation, the method comprising: estimating an amount of a protein in a load solution; and determining a flow rate for the frontal chromatography operation based on a target residence time, wherein the flow rate is in the range of 50 cm/hr to 250 cm/hr and a bed height for a frontal chromatography medium is a constant value in the range of 5 cm to 30 cm.

In some embodiments, a bed diameter for the frontal chromatography medium is held constant.

In some embodiments, the bed diameter is greater than 1 cm, greater than 2 cm, greater than 3 cm, greater than 4 cm, greater than 5 cm, greater than 10 cm, greater than 15 cm, greater than 20 cm, greater than 25 cm, greater than 30 cm, greater than 35 cm, greater than 40 cm, or greater than 45 cm. In some embodiments of methods described herein, the bed diameter is greater than 80 cm, greater than 100 cm, or greater than 120 cm.

In some embodiments, the bed diameter is greater than 1 cm. In some embodiments, the bed diameter is greater than 10 cm.

In some embodiments, the bed diameter is 1 cm, 2 cm, 3 cm, 4 cm, 5 cm, 10 cm, 15 cm, 20 cm, 25 cm, 30 cm, 35 cm, 40 cm, or 45 cm. In some embodiments, the bed diameter is 50 cm, 60 cm, 70 cm, 80 cm, 90 cm, 100 cm, 110 cm, 120 cm, 130 cm, 140 cm, 150 cm, 160 cm, 170 cm, 180 cm, 190, or 200 cm.

In some embodiments, the bed diameter is in the range of 1 cm to 20 cm. In some embodiments, the bed diameter is 1 cm, 10 cm, or 20 cm. In some embodiments, the bed diameter is 1 cm. In some embodiments, the bed diameter is 10 cm. In some embodiments, the bed diameter is 20 cm.

In some embodiments, the flow rate is in the range of 100 cm/hr to 250 cm/r. In some embodiments, the flow rate is in the range of 100 cm/hr to 200 cm/hr.

In some embodiments, the flow rate is 133 cm/hr or 200 cm/hr. In some embodiments, the flow rate is 133 cm/hr. In some embodiments, the flow rate is 200 cm/hr.

In some embodiments, the bed height is a constant value in the range of 10 cm to 20 cm.

In some embodiments, the frontal chromatography medium is a cation exchange chromatography resin. In some embodiments, the frontal chromatography medium is a cation exchange chromatography resin comprising tentacle ligands (e.g., Eshmuno® CP-FT resin). In some embodiments, the frontal chromatography medium is packed in a chromatography column. In some embodiments, the frontal chromatography medium is a cation exchange chromatography resin and is packed in a chromatography column. In some embodiments, the frontal chromatography medium is a cation exchange chromatography resin comprising tentacle ligands (e.g., Eshmuno® CP-FT resin) and is packed in a chromatography column.

In some embodiments, the amount of protein in the load solution is estimated based on monitoring performed in real-time, near real-time, and/or offline. In some embodiments, the amount of protein in the load solution is estimated based on historical data. In some embodiments, the estimate is performed after one or more cycles. In some embodiments, the estimate is performed after one or more batches.

### BRIEF DESCRIPTION OF THE DRAWINGS

FIG. 1 is a schematic depicting the basic operating sequence for a frontal CEX chromatography resin from equilibration to storage.

FIG. 2 is a graph depicting the maximum ΔP (change in column pressure) observed during isocratic strip cleaning (50 mM acetate, 1M CaCl₂, pH 5.0) of a frontal CEX chromatography column used to purify a mAb over multiple cycles with 750 g/L-r column loading and load material with pH 5.0 and 5.3 mS/cm conductivity.

FIG. 3 shows the ΔP profile and UV absorbance (A280, absorbance at 280 nm) for bench-scale equilibration, frontal load, wash, non-denaturing strip, and denaturing strip of a CEX resin cleaned using a non-denaturing linear salt gradient. Specifically, following the wash, the resin was subjected to a non-denaturing cleaning step comprising a linear salt gradient (0-100% Buffer B (Buffer A: 50 mM acetate, 0 M NaCl, pH 5.0, Buffer B: 50 mM acetate, 0.5 M NaCl pH 5.0) greater than or equal to 7 CV, followed by a denaturing step using 1 M NaOH. The resin was then stored in 0.2 M NaOH. Absorbance (280 nM) is shown by the thin line. AP is shown by the thick line.

FIG. 4 shows cycle-to-cycle pre-column pressure using linear salt gradient cleaning over multiple cycles (100 cycles).

FIG. 5 shows the step yield as a function of column loading for a frontal CEX chromatography step used to purify a mAb. The data include both pilot-scale and bench-scale chromatography operations, with load material for bench-scale data obtained from pilot-scale bioreactors (500 L to 2000 L).

FIG. 6 is a graph depicting the predicted step yield for a frontal CEX step performed at four model facilities with varying facility constraints using different target bed heights.

### DETAILED DESCRIPTION

In biologics manufacturing operations, polishing chromatography may be used to remove a diverse array of product- and process-related impurities, including high molecular weight (HMW) and low molecular weight (LMW) product-related species. For example, cation-exchange (CEX) chromatography operated in bind and elute mode is widely used for its high degree of chromatographic resolution for the product species of interest, robust clearance of host cell impurities and viruses, and reasonably high yields. However, removal of key impurities such as HMW product-related species generally decreases with increased loading density. The load capacity of polishing chromatography operations can constrain the throughput of the overall downstream process. In such cases, strategies to process an entire product batch within the facility's target run rate may require scaling up the diameter of the rate-limiting chromatography operation and/or parallel processing of the lot on multiple columns, both of which are expensive mitigations that may not be feasible within a facility's footprint and operating parameters. Additionally, these strategies can increase buffer volume requirements, along with energy and water consumption, and may provide less flexibility to accommodate improvements in upstream titer and/or productivity.

In bind and elute mode, a chromatography medium having some type of affinity for the product of interest is used. The product binds to the chromatography medium during loading. The bound product is then eluted and collected as the product pool. Certain impurities, such as product-related species, may bind to the chromatography medium during loading with greater or lesser affinity than the product of interest. Various strategies are employed to purify the desired product from co-binding impurities, including use of pre-elution wash(es) to desorb unwanted low-affinity impurities, gradient elution which gradually changes the composition of the mobile phase in order to improve the separation between the product of interest and impurities during elution, and designing the pool collection criteria to collect the product of interest without introducing low-affinity or high-affinity impurities.

To achieve increased loading density during a polishing chromatography step, alternate chromatographic operating modes such as flow-through chromatography, weak partitioning chromatography, overload chromatography, and frontal loading chromatography may be employed. In flow-through mode, chromatography medium and operation conditions favor binding the impurities and contaminants while the product of interest flows through, such that the effluent is enriched in the product of interest. In overload chromatography, the product is loaded onto the chromatography material beyond the dynamic binding capacity of the material. Both weak partitioning and frontal loading chromatography modes rely on competitive binding to the chromatography medium between the components in the load to separate the product of interest from impurities and contaminants. For example, frontal loading mode is characterized by continuous loading under conditions in which all the components in the load feed initially bind the chromatography medium. In an ideal operation, as loading proceeds, the bound components are displaced in order of increasing affinity for the chromatography medium (i.e., the stationary phase) until the medium is heavily saturated with those components of higher binding affinity. Illustratively, in a frontal loading mode cation exchange chromatography (CEX) operation designed to bind positively charged species, the product of interest (i.e., monomer) and low molecular weight (LMW) product-related impurities (i.e., truncated proteins expressed during culture, degraded proteins, and enzymatically clipped proteins) are usually retained less strongly and are quickly displaced by more positively charged components. The product of interest and any LMW impurities flow through into the effluent, which may be diverted to a downstream unit operation or collected as a product pool. A chromatography operation making use of frontal loading mode can remove HMW product-related species such as dimers, oligomers, and higher-order aggregates at high loading density, along with process-related impurities, such as host cell proteins (HCP), that have a higher binding affinity and are retained by the chromatography medium. As loading progresses, the effluent is enriched in the product of interest as the chromatography medium becomes more saturated by the higher affinity impurities. Beyond this saturation point, additional impurities loaded onto the resin are expected to flow through into the effluent along with the product of interest. Therefore, the saturation point for critical impurities designed to bind during the frontal loading chromatography step (such as, e.g., HMW product-related impurities) typically represents the practical upper limit for loading density.

High loading of chromatography medium in frontal loading mode can provide over an order of magnitude higher HMW impurity removal than loading in bind and elute mode. Higher loading may be desirable because of improved utilization of the chromatography medium (e.g., per unit volume of a packed chromatography column), reduction in buffer consumption, and improvements in productivity. The extent of these improvements depends on process parameters such as the allowable loading densities, the operating flow rate, bed height, and the feed stream concentration.

However, one limitation of frontal chromatography compared to bind and elute chromatography is that the loading and the yield of a frontal step are highly correlated. With increased loading beyond saturation by the product of interest, the fraction of the loaded product that does not bind to the resin increases, increasing the recovered product yield from a frontal chromatography operation. Conversely, the relative amount of product that remains bound to the resin decreases resulting in a non-linear yield versus loading relationship. As such, the overall process yield can be sensitive to anything that impacts the amount of protein available for loading, such as increased/decreased product titer in the load buffer.

Another limitation of frontal chromatography is the challenge high loading presents for chromatography reuse. Chromatography reuse is a process in which a chromatography medium is cleaned and/or regenerated for further use in the purification of the same product or a different product. High load densities may result in saturation or near saturation of the chromatography medium with impurities such as host-cell proteins, potentially leading to high column pressures during regeneration/wash and/or sanitization phases for the chromatography medium. Insufficient cleaning and/or high column pressures may reduce the number of cycles in which a chromatography medium can be reused and/or may impact its performance in subsequent cycles.

### NON-LIMITING EMBODIMENTS

Non-limiting embodiments of the present disclosure include, but are not limited to, the following:

E1. A method of cleaning a chromatography medium for reuse, comprising:
loading a composition comprising a protein and at least one impurity onto the chromatography medium;
collecting fractions comprising the protein; and
cleaning the chromatography medium using a linear salt gradient.

E2. The method of E1, wherein the loading density exceeds the dynamic binding capacity of the chromatography medium for the protein.

E3. The method of E1 or E2, wherein the loading density is at least 200 g/L-r.

E4. The method of any one of E1 to E3, wherein the loading density is at least 1000 g/L-r (e.g., in the range of 1000 g/L-r to 1500 g/L-r).

E5. The method of any one of E1 to E4, wherein the chromatography medium is used in frontal mode for the purification of the protein.

E6. The method of any one of E1 to E5, wherein the chromatography medium is loaded at or near saturation with the at least one impurity.

E7. The method of any one of E1 to E6, wherein the at least one impurity comprises one or more high molecular weight species of the protein.

E8. The method of any one of E1 to E7, wherein the linear salt gradient comprises an increase in salt concentration from less than 50 mM to 500 mM.

E9. The method of any one of E1 to E7, wherein the linear salt gradient comprises an increase in salt concentration from 0 mM or 20 mM to 500 mM.

E10. The method of any one of E1 to E9, wherein the linear salt gradient is an NaCl gradient, a KCl gradient, a CaCl₂ gradient, or an Na₂SO₄ gradient.

E11. The method of any one of E1 to E10, wherein the linear salt gradient is an NaCl gradient.

E12. The method of any one of E1 to E11, wherein the linear salt gradient is generated using at least two buffers.

E13. The method of E12, wherein each of the at least two buffers used to generate the linear salt gradient is independently selected from acetate buffers, phosphate buffers, Tris buffers, and 2-(N-morpholino) ethanesulfonic acid buffers.

E14. The method of E12 or E13, wherein each of the at least two buffers used to generate the linear salt gradient comprises acetate.

E15. The method of any one of E12 to E14, wherein each of the at least two buffers used to generate the linear salt gradient comprises acetate at a concentration of 50 mM.

E16. The method of any one of E12 to E15, wherein the pH of each of the at least two buffers used to generate the linear salt gradient is greater than 3.6.

E17. The method of any one of E1 to E16, wherein the pH of each of the at least two buffers used to generate the linear salt gradient is less than 5.6.

E18. The method of any one of E1 to E11, wherein the linear salt gradient is generated by Buffer A and Buffer B, wherein Buffer A comprises 50 mM acetate and 0 mM or 20 mM sodium chloride at a pH value of 5.0±0.1 and Buffer B comprises 50 mM acetate and 500 mM sodium chloride at a pH value of 5.0±0.1.

E19. The method of E18, wherein the linear salt gradient is generated using Buffer A and Buffer B in a gradient from 100% Buffer A (0% Buffer B) to 0% Buffer A (100% Buffer B), or 90% Buffer A (10% Buffer B) to 10% Buffer A (90% Buffer B), or 80% Buffer A (20% Buffer B) to 20% Buffer A (80% Buffer B).

E20. The method of any one of E1 to E19, wherein the slope of the linear salt gradient is less than or equal to 0.07 M salt/medium volume buffer.

E21. The method of any one of E1 to E20, wherein the gradient length is at least 7 medium volumes.

E22. The method of any one of E1 to E21, wherein the cleaning using the linear salt gradient is non-denaturing.

E23. The method of any one of E1 to E22, wherein the cleaning using the linear salt gradient is performed after at least one cycle.

E24. The method of any one of E1 to E22, wherein the cleaning using the linear salt gradient is performed after at least one batch.

E25. The method of any one of E1 to E22, wherein the cleaning using the linear salt gradient is performed prior to storage.

E26. The method of any one of E1 to E25, further comprising cleaning the chromatography medium using an isocratic cleaning process using a denaturing solution.

E27. The method of E26, wherein the denaturing solution comprises sodium hydroxide.

E28. The method of E26 or E27, wherein the denaturing solution comprises sodium hydroxide at a concentration of 1 M.

E29. The method of any one of E26 to E28, wherein 3 medium volumes of the denaturing solution are passed through the chromatography medium.

E30. The method of any one of E26 to E29, wherein the cleaning using the denaturing solution is performed after at least one cycle.

E31. The method of any one of E26 to E29, wherein the cleaning using the denaturing solution is performed after at least one batch.

E32. The method of any one of E26 to E29, wherein the cleaning using the denaturing solution is performed prior to storage.

E33. The method of E25 or E32, wherein the chromatography medium is stored in a storage solution comprising sodium hydroxide at a concentration in the range of 0.1 M to 0.2 M.

E34. A method for controlling operating pressure during cleaning of a chromatography medium for reuse, comprising:
cleaning the chromatography medium using a linear salt gradient; and
cleaning the chromatography medium using a denaturing solution.

E35. A method for purifying a protein from a composition comprising the protein and at least one impurity, comprising:
equilibrating a chromatography medium using an equilibration buffer;
loading the composition comprising the protein and the at least one impurity onto the chromatography medium in frontal mode at a loading density of at least 500 g/L-r;
collecting fractions comprising the protein;
passing less than 2 medium volumes of a post-load wash solution through the chromatography medium and adding the eluate to the fractions;
cleaning the chromatography medium using a linear salt gradient; and
cleaning the chromatography medium using a denaturing solution.

E36. The method of E35, wherein fraction collection begins at 0.5 OD, A280 (absorbance at 280 nm).

E37. The method of E35 or E36, wherein fraction collection is stopped after less than or equal to one medium volume of the post-load wash solution is passed through the chromatography medium.

E38. The method of any one of E35 to E37, wherein the equilibration buffer does not contain salt.

E39. A method for purifying a protein from a composition comprising the protein and at least one impurity, comprising:
initiating a cell culture in a bioreactor;
culturing cells engineered to express the protein;
harvesting cell culture broth comprising the protein;
loading the harvested cell culture broth onto an affinity chromatography medium in bind and elute mode;
collecting affinity chromatography elution fractions comprising the protein;
loading the affinity chromatography elution fractions onto a cation exchange (CEX) chromatography medium at a loading density of at least 500 g/L-r in frontal mode;
collecting CEX chromatography fractions comprising the protein;
cleaning the CEX chromatography medium using a linear salt gradient; and
cleaning the CEX chromatography medium using a denaturing solution.

E40. The method of E39, wherein the affinity chromatography medium is a Protein A affinity chromatography medium.

E41. The method of E39 or E40, wherein the affinity chromatography elution fractions are subjected to low pH viral inactivation and optional neutralization post-viral inactivation.

E42. The method of any one of E39 to E41, wherein the protein is eluted from the affinity chromatography medium at a pH suitable for viral inactivation.

E43. The method of any one of E39 to E42, wherein the protein is eluted from the affinity chromatography medium at a pH of less than or equal to 4.0.

E44. The method of E42 or E43, wherein the affinity chromatography elution fractions are loaded onto the CEX chromatography medium in a loading time sufficient to achieve viral inactivation.

E45. The method of E44, wherein the loading time is at least 30 minutes.

E46. The method of any one of E34 to E45, wherein the denaturing solution is an used in an isocratic cleaning process.

E47. The method of any one of E1 to E38, wherein the chromatography medium is a cation exchange (CEX) chromatography medium.

E48. The method of any one of E1 to E47, wherein the chromatography medium is packed in a chromatography column.

E49. A method of cleaning a chromatography medium for reuse, comprising:
loading a composition comprising a protein and at least one impurity onto the chromatography medium, wherein the loading density exceeds the dynamic binding capacity of the chromatography medium for the protein;
collecting fractions comprising the protein; and
cleaning the chromatography medium using a linear salt gradient.

E50. The method of E49, wherein the chromatography medium is a cation exchange (CEX) chromatography resin.

E51. The method of E49 or E50, wherein the chromatography medium is packed in a chromatography column.

E52. The method of any one of E49 to E51, wherein the loading density is at least 200 g/L-r.

E53. The method of any one of E49 to E52, wherein the loading density is at least 1000 g/L-r (e.g., in the range of 1000 g/L-r to 1500 g/L-r).

E54. The method of any one of E49 to E53, wherein the chromatography medium is loaded at or near saturation with the at least one impurity.

E55. The method of any one of E49 to E54, wherein the at least one impurity comprises one or more high molecular weight species of the protein.

E56. The method of any one of E49 to E55, wherein the linear salt gradient comprises an increase in salt concentration from less than 50 mM to 500 mM.

E57. The method of any one of E49 to E56, wherein the linear salt gradient is an NaCl gradient, a KCl gradient, a CaCl₂ gradient, or an Na₂SO₄ gradient.

E58. The method of any one of E49 to E57, wherein the linear salt gradient is an NaCl gradient.

E59. The method of any one of E49 to E58, wherein the linear salt gradient is generated using at least two buffers, wherein each of the at least two buffers used to generate the linear salt gradient is independently selected from acetate buffers, phosphate buffers, Tris buffers, and 2-(N-morpholino) ethanesulfonic acid buffers.

E60. The method of E59, wherein each of the at least two buffers used to generate the linear salt gradient comprises acetate.

E61. The method of E59 or E60, wherein the pH of each of the at least two buffers used to generate the linear salt gradient is greater than 3.6 and less than 5.6.

E62. The method of any one of E49 to E58, wherein the linear salt gradient is generated by Buffer A and Buffer B, wherein Buffer A comprises 50 mM acetate and 0 mM or 20 mM sodium chloride at a pH value of 5.0±0.1 and Buffer B comprises 50 mM acetate and 500 mM sodium chloride at a pH value of 5.0±0.1.

E63. The method of E62, wherein the linear salt gradient is generated using Buffer A and Buffer B in a gradient from 100% Buffer A (0% Buffer B) to 0% Buffer A (100% Buffer B), or 90% Buffer A (10% Buffer B) to 10% Buffer A (90% Buffer B), or 80% Buffer A (20% Buffer B) to 20% Buffer A (80% Buffer B).

E64. The method of any one of E49 to E63, wherein the slope of the linear salt gradient is less than or equal to 0.07 M salt/medium volume buffer.

E65. The method of any one of E49 to E64, wherein the gradient length is at least 7 medium volumes.

E66. The method of any one of E49 to E65, wherein the cleaning using the linear salt gradient is non-denaturing.

E67. The method of any one of E49 to E66, further comprising cleaning the chromatography medium using an isocratic cleaning process using a denaturing solution.

E68. The method of E67, wherein the denaturing solution comprises sodium hydroxide.

E69. The method of any one of E49 to E68, wherein the method enables reuse of the chromatography medium for multiple cycles.

E70. The method of any one of E49 to E68, wherein the method enables reuse of the chromatography medium for at least 25 cycles.

E71. A method for purifying a protein from a composition comprising the protein and at least one impurity, comprising:
equilibrating a chromatography medium using an equilibration buffer;
loading the composition comprising the protein and the at least one impurity onto the chromatography medium in frontal mode at a loading density of at least 500 g/L-r;
collecting fractions comprising the protein;
passing less than 2 medium volumes of a post-load wash solution through the chromatography medium and adding the eluate to the fractions;
cleaning the chromatography medium using a linear salt gradient; and
cleaning the chromatography medium using a denaturing solution.

E72. The method of E71, wherein fraction collection begins at 0.5 OD, A280 (absorbance at 280 nm).

E73. The method of E71 or E72, wherein fraction collection is stopped after less than or equal to one medium volume of the post-load wash solution is passed through the chromatography medium.

E74. The method of any one of E71 to E73, wherein the equilibration buffer does not contain salt.

E75. The method of any one of E71 to E74, wherein the denaturing solution is an used in an isocratic cleaning process.

E76. The method of any one of E71 to E75, wherein the chromatography medium is a cation exchange (CEX) chromatography resin.

E77. The method of any one of E71 to E76, wherein the chromatography medium is packed in a chromatography column.

E78. A method for improving step yield for a frontal chromatography operation, comprising: estimating an amount of a protein in a load solution; and determining a bed height for a frontal chromatography medium based on a target residence time, wherein the flow rate is a constant value in the range of 50 cm/hr to 250 cm/hr.

E79. A method for improving step yield for a frontal chromatography operation, comprising: estimating an amount of a protein in a load solution; and determining a flow rate for the frontal chromatography operation based on a target residence time, wherein the flow rate is in the range of 50 cm/hr to 250 cm/hr and a bed height for a frontal chromatography medium is a constant value in the range of 5 cm to 30 cm.

E80. The method of E78 or E79, wherein a bed diameter for the frontal chromatography medium is held constant.

E81. The method of any one of E78 to E80, wherein the amount of protein in the load solution is estimated based on monitoring performed in real-time, near real-time, and/or offline.

E82. The method of any one of E78 to E80, wherein the amount of protein in the load solution is estimated based on historical data.

E83. The method of any one of E78 to E82, wherein the estimate is performed after one or more cycles.

E84. The method of any one of E78 to E82, wherein the estimate is performed after one or more batches.

E85. The method of any one of E78 to E84, wherein the bed diameter is in the range of 1 cm to 20 cm.

E86. The method of any one of E78 to E85, wherein the bed diameter is 1 cm, 10 cm, or 20 cm.

E87. The method of any one of E78 to E86, wherein the bed diameter is 1 cm.

E88. The method of any one of E78 to E86, wherein the bed diameter is 10 cm.

E89. The method of any one of E78 to E86, wherein the bed diameter is 20 cm.

E90. The method of any one of E78 to E89, wherein the frontal chromatography medium is a cation exchange chromatography medium.

E91. The method of any one of E78 to E90, wherein the frontal chromatography medium is a cation exchange chromatography resin.

E92. The method of any one of E78 to E91, wherein the frontal chromatography medium is a cation exchange chromatography resin comprising tentacle ligands.

E93. The method of any one of E78 to E92, wherein the frontal chromatography medium is packed in a column.

E94. The method of any one of E1 to E93, wherein the protein is an antibody.

E95. The method of any one of E1 to E94, wherein the protein is a monoclonal antibody.

E96. The method of any one of E1 to E95, wherein the protein is an IgG2 antibody.

### Definitions

Where a range of values is provided herein, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges also encompassed within the disclosure, subject to any specifically excluded limit in the stated range. Where the stated range includes one or both of the limits, ranges excluding either or both of those included limits are also included in the disclosure.

As used herein, the terms “a” and “an” mean “one or more” unless specifically indicated otherwise. Additionally, “one or more” and “at least one” are used interchangeably herein. Furthermore, unless otherwise required by context, singular terms include pluralities and plural terms include the singular.

Throughout this specification and the claims which follow, unless the context requires otherwise, the term “comprise,” and variations such as “comprises” and “comprising,” will be understood to imply the inclusion of a stated integer or step or group of integers or steps but not the exclusion of any other integer or step or group of integer or step. When used herein, the term “comprising” can be substituted with the term “containing” or “including” or sometimes when used herein with the term “having”.

As used herein, “consisting of” excludes any element, step, or ingredient not specified in the embodiment feature or claim element. As used herein, “consisting essentially of” does not exclude materials or steps that do not materially affect the basic and novel characteristics of the embodiment feature or claim element.

In each instance herein, any of the terms “comprising,” “consisting essentially of,” “consisting of,” and their variations may be replaced with any of the other two terms or their variations.

As used herein, the term “affinity chromatography” (also referred to as “capture chromatography”) refers to a chromatography operation in which a biomolecule (e.g., a recombinant protein) is separated from a mixture based on a selective interaction between the biomolecule and another substance (i.e., a ligand). Affinity chromatography is commonly used in biomanufacturing processes to isolate and concentrate desired recombinant proteins from harvested cell culture fluid. In a typical affinity chromatography operation, a biomolecule in a moving phase selectively binds to or otherwise interacts with a stationary phase while the rest of the moving phase passes through the chromatography material. The biomolecule is then eluted from the stationary phase by changing the conditions in a manner that reduces the affinity between the ligand and the biomolecule. Non-limiting examples of affinity chromatography materials include Protein A, Protein G, Protein A/G, and Protein L materials. Additionally, immobilized metal affinity chromatography (IMAC) can be used to capture proteins that have or have been engineered to have affinity for metal ions.

In some embodiments, protein A affinity chromatography may be employed to capture a protein of interest. Protein A ligands are highly selective for a wide range of proteins containing an antibody Fc region and provide robust removal of process-related impurities with high target protein yields. Commercially available protein A materials include, but are not limited to, MABSELECT™ SURE Protein A, Protein A Sepharose FAST FLOW™, MABSELECT™ PrismA (Cytiva, Marborough, MA), PROSEP-A™ (Merck Millipore, U.K), TOYOPEARL® HC-650F Protein A (TosoHass Co., Philadelphia, PA), and AP Plus, Purolite, King of Prussia, PA).

As used herein, “bed height” is the height of chromatography medium used.

As used herein, “bed diameter” is the diameter of chromatography medium used. In some embodiments of methods described herein, the bed diameter is greater than 1 cm, greater than 2 cm, greater than 3 cm, greater than 4 cm, greater than 5 cm, greater than 10 cm, greater than 15 cm, greater than 20 cm, greater than 25 cm, greater than 30 cm, greater than 35 cm, greater than 40 cm, or greater than 45 cm. In some embodiments, the bed diameter is 1 cm, 2 cm, 3 cm, 4 cm, 5 cm, 10 cm, 15 cm, 20 cm, 25 cm, 30 cm, 35 cm, 40 cm, or 45 cm. In some embodiments of methods described herein, the bed diameter is greater than 80 cm, greater than 100 cm, or greater than 120 cm. In some embodiments, the bed diameter is 50 cm. 60 cm, 70 cm, 80 cm, 90 cm, 100 cm, 110 cm, 120 cm, 130 cm, 140 cm, 150 cm, 160 cm, 170 cm, 180 cm, 190, or 200 cm. In some embodiments, bed diameter is determined based on the amount of protein or contaminants in the load.

As used herein, the term “bioreactor” means any vessel useful for the growth of a mammalian cell culture. The term “fermenter,” as used herein, refers to any vessel useful for the growth of a bacterial cell culture and typically contains a more rigorous agitator and increased gas flow relative to a vessel used for the growth of a mammalian cell culture.

Non-limiting examples of bioreactors include stirred tank, airlift, fiber, microfiber, hollow fiber, ceramic matrix, fluidized bed, fixed bed, and/or spouted bed bioreactors. In some embodiments, an example bioreactor can perform one or more (e.g., one, two, three, all) of the following steps: feeding of nutrients and/or carbon sources, injection of suitable gas (such as, e.g., oxygen), inlet and outlet flow of fermentation or cell culture medium (e.g., perfusion of fresh cell culture medium in and removal of spent cell culture medium), separation of gas and liquid phases, maintenance of temperature, maintenance of oxygen and CO₂ levels, maintenance of pH level, agitation (e.g., stirring), and/or cleaning/sterilizing. Unless otherwise indicated by context, a bioreactor can be suitable for batch, semi fed-batch, fed-batch, perfusion, and/or continuous fermentation processes. Any suitable bioreactor diameter can be used. Unless otherwise indicated by context, in some embodiments, the bioreactor can have a volume between 100 mL and 50,000 L. Unless otherwise indicated, a bioreactor can be of any size so long as it is useful for the culturing of cells; typically, a bioreactor is sized appropriate to the volume of cell culture being grown inside of it. In non-limiting embodiments and unless otherwise indicated by context, a bioreactor may be at least 1 liter (L) or may be 2, 5, 10, 50, 100, 200, 250, 500, 1,000, 1500, 2000, 2,500, 5,000, 8,000, 10,000, 12,000 liters, 20,000 L or more, or any volume in between. The internal conditions of the bioreactor, including, but not limited to, pH, dissolved oxygen concentration, and temperature, can be controlled during the culturing period. Those of ordinary skill in the art will be aware of, and will be able to select, suitable bioreactors for use in the methods disclosed herein based on the relevant considerations.

As used herein, a “buffer” refers to a buffered solution that resists changes in pH by the action of its acid-base conjugate components.

As used herein, the term “cell culture” or “culture” refers to the growth and propagation of cells outside of a multicellular organism or tissue. Suitable culture conditions for mammalian and bacterial cells are known in the art. (See, e.g., Animal cell culture: A Practical Approach, D. Rickwood, ed., Oxford University Press, New York (1992).) Mammalian cells may be cultured in suspension or while attached to a solid substrate. In some embodiments, fluidized bed bioreactors, hollow fiber bioreactors, roller bottles, shake flasks, and/or stirred tank bioreactors, with or without microcarriers, may be used for cell culture. In some embodiments, 500 L to 2000 L bioreactors are used for cell culture (e.g., as part of a seed train). In some embodiments, 1000 L to 2000 L bioreactors are used for cell culture (e.g., as part of a seed train).

As used herein, the term “cell culturing medium” (also referred to as “media,” “culture medium,” “cell culture media,” “tissue culture media,” and the like) refers to any nutrient solution used for growing cells, e.g., bacterial or mammalian cells. Cell culturing medium generally provides one or more of the following components: an energy source (e.g., in the form of a carbohydrate, such as, e.g., glucose); one or more essential amino acids (e.g., all essential amino acids; the twenty basic amino acids plus cysteine); vitamins and/or other organic compounds typically required at low concentrations; lipids or free fatty acids; and trace elements, such as, e.g., inorganic compounds or naturally occurring elements that are typically required at very low concentrations, such as, e.g., concentrations in the micromolar range. As used herein, cell culturing medium encompasses nutrient solutions that are typically employed in and/or are known for use with any cell culture process, including, but not limited to, batch, extended batch, fed-batch, intensified, and/or perfusion or continuous culturing of cells.

As used herein, the term “cell density” refers to the number of cells in a given volume of culture medium. “Viable cell density” refers to the number of live cells in a given volume of culture medium, as determined by standard viability assays (such as, e.g., a trypan blue dye exclusion method). As used herein, the term “packed cell volume” (PCV), also referred to as “percent packed cell volume” (% PCV), is the ratio of the volume occupied by the cells, to the total volume of cell culture, expressed as a percentage (see Stettler, et al., (2006) Biotechnol Bioeng. December 20: 95 (6): 1228-33). Packed cell volume is a function of cell density and cell diameter; increases in packed cell volume could arise from increases in cell density or cell diameter or both. Packed cell volume is a measure of the solid content in the cell culture. Since host cells vary in size and cell cultures also contain dead and dying cells and other cellular debris, packed cell volume can describe with a greater degree of accuracy the solid content within a cell culture.

As used herein, a “column” or “chromatography column” refers to a device for separating components in a solution. The column consists of a stationary phase that adsorbs and separates components passing through in the liquid mobile phase. A solid adsorbent, such as a chromatography resin, is packed in a glass or metal column. Filtration-based adsorbents, such as membranes, and monoliths can serve as the stationary phase. The liquid phase comprises the components to be separated. (e.g., the product of interest, impurities, contaminants, and the like) in a buffer solution. As used herein, the “medium volume” of a chromatography medium is the total volume of chromatography medium used. For example, when the chromatography medium is packed in a column, the medium volume is the total volume of chromatography medium in the column, which may alternatively be referred to as the column volume. In all embodiments of the present disclosure, chromatography media may be packed in a chromatography column and references to “medium volumes” or “MVs” may be replaced by “column volumes” or “CVs.”

As used herein, a “cycle” comprises the steps from loading a chromatography medium with a loading solution comprising the product of interest and at least one impurity to collecting the purified product of interest exiting the chromatography column in the effluent, as well as post-collection cleaning and equilibration steps. There may be one or more cycles in a batch. As used herein, a “batch” comprises all the chromatography cycles necessary to process a fixed quantity of fluid containing the product of interest. The fluid may be derived from an elution pool or effluent from a cell culture harvest, affinity or polishing chromatography, viral inactivation, neutralization, depth filtration, virus filtration, ultrafiltration, diafiltration, or other upstream operation.

As used herein, the term “dynamic binding capacity,” in reference to a chromatography medium, refers to the amount of product, e.g., protein, the chromatography medium will bind under actual flow conditions before significant breakthrough of unbound product occurs.

As used herein, “load” refers to a composition loaded onto a chromatography medium.

As used herein, “loading”, “loading density”, “load factor”, and “column loading” refer to the quantity of product of interest, expressed in grams of component per liter of resin (g/L-r). The chromatography medium may be loaded with an effluent stream or pool containing the product of interest and at least one impurity. Additional buffers may be added to the effluent stream or pool such that the final load is at a desired volume, pH, conductivity, and/or formulation, for example.

As used herein, the term “expression vector” or “expression construct” refers to a recombinant DNA molecule containing a desired coding sequence and appropriate nucleic acid control sequences necessary for the expression of the operably linked coding sequence in a particular host cell, e.g., a mammalian host cell. Vectors can include viral vectors, nonepisomal mammalian vectors, plasmids, and other non-viral vectors. An expression vector can include sequences that affect or control transcription, translation, and, if introns are present, affect RNA splicing of a coding region operably linked thereto. “Operably linked” means that the components to which the term is applied are in a relationship that allows them to carry out their inherent functions. For example, a control sequence, e.g., a promoter, in a vector that is “operably linked” to a protein coding sequence is arranged such that normal activity of the control sequence leads to transcription of the protein coding sequence resulting in recombinant expression of the encoded protein.

As used herein, “fed-batch culture” refers to a form of suspension culture, specifically a method of culturing cells in which additional components are provided to the culture at a time or times subsequent to the beginning of the culture process. The provided components typically comprise nutritional supplements for the cells which have been depleted during the culturing process. Additionally or alternatively, the additional components may include supplementary components (such as, e.g., a cell-cycle inhibitory compound). In some embodiments, fed-batch cell culture medium formulations may be richer or more concentrated than basal cell culture medium formulations, which contain components essential for cell survival and growth and are typically used to initiate a cell culture. A fed-batch culture may be stopped at some point, and the cells and/or components in the medium may be harvested and optionally purified.

As used herein, a “growth phase” of a cell culture refers to the period of exponential cell growth (i.e., the log phase) where cells are generally rapidly dividing.

As used herein, the term “harvested cell culture fluid” or “harvested cell culture broth” refers to a solution which has been processed by one or more operations to separate cells, cell debris, or other large particulates from a protein of interest. Such operations include, but are not limited to, cooling, flocculation, acidification, centrifugation, neutralization, acoustic wave separation, and various forms of filtration (e.g., depth filtration, microfiltration, ultrafiltration, tangential flow filtration, and alternating tangential flow filtration). Harvested cell culture fluid includes cell culture lysates as well as cell culture supernatants. The harvested cell culture fluid may be further clarified to remove fine particulate matter and soluble aggregates by filtration with a membrane having a pore size between about 0.1 μm and about 0.5 μm, such as, e.g., a membrane having a pore size of about 0.22 μm.

As used herein, a “host cell” refers to a cell that has been transformed, or is capable of being transformed, with a nucleic acid and thereby expresses a gene of interest. The term includes the progeny of the parent cell, whether or not the progeny is identical in morphology or in genetic make-up to the original parent cell, so long as the gene of interest is present. A host cell that comprises a nucleic acid encoding a recombinant protein, e.g., operably linked to at least one expression control sequence (e.g. promoter or enhancer), is a “recombinant host cell.” A host cell, when cultured under appropriate conditions, may synthesize a recombinant protein that can subsequently be collected from the culture medium (if the host cell secretes it into the medium) or directly from the host cell producing it (if it is not secreted).

As used herein, “high molecular weight” or “HMW” species of a protein of interest refer to dimers, oligomers, and aggregates of the protein that have a molecular weight greater than the molecular weight of the intact, fully assembled form of the protein.

As used herein, the term “impurity” refers to a component other than the protein of interest, along with its associated buffer components. Impurities include, but are not limited to, process- and product-related impurities, such as, e.g., host cell proteins, leached resin materials (such as, e.g., leached protein A), nucleic acids, HMW species of the protein, LMW species of the protein, endotoxins, viral contaminants, cell culture media components, and the like.

As used herein, “low molecular weight” or “LMW” species of a protein of interest refer to fragments, truncated forms, or other incomplete variants of the protein that have a molecular weight less than the molecular weight of the intact, fully assembled form of the recombinant protein. LMW species can include, but are not limited to, proteolytic fragments, truncated forms resulting from cellular expression of mRNA splice variants, and single component polypeptides in the case of multi-polypeptide chain proteins (e.g., light chain or heavy chain only species when the recombinant protein is an antibody).

As used herein, a “perfusion” cell culture medium refers to a cell culture medium that is typically used in cell cultures that are maintained by perfusion or continuous culture methods and is sufficiently complete to support the cell culture during this process. In some embodiments, perfusion cell culture medium formulations may be richer or more concentrated than base cell culture medium formulations to accommodate the method used to remove the spent medium. In some embodiments, perfusion cell culture medium may be used during both the growth and production phases.

As used herein, the term “polishing chromatography” refers to a chromatography operation performed after a capture or affinity chromatography operation to remove remaining impurities and obtain a more highly purified composition and/or protein. Common impurities removed during polishing steps include, but are not limited to, product-related impurities (e.g., HMW and LMW species), host cell proteins, DNA, leached protein A, viral contaminants, and endotoxins. In addition, typical chromatography techniques used for polishing include, but are not limited to, ion exchange chromatography (IEX), hydrophobic interaction chromatography (HIC), and multimodal (or mixed mode) chromatography (MMC).

As used herein, “anion exchange chromatography” (AEX) refers to a form of ion exchange chromatography performed on a solid phase medium (e.g., resin or membrane) that is positively charged and has the capacity to exchange free anions with anions in an aqueous solution passed over or through the solid phase. AEX chromatography is used, for example, for viral clearance and impurity removal. Commercially available anion exchange media include, but are not limited to, sulphopropyl (SP) immobilized on agarose (e.g., Source 15 Q, Capto™ Q, Q-SEPHAROSE FAST FLOW™ (Cytiva), FRACTOGEL EMD TMAE™, FRACTOGEL EMD DEAE™, (EMD Merck), TOYOPEARL® Super Q® and TOYOPEARL® NH2-750F (Tosoh Bioscience), POROS HQ™, and POROS XQ™ (ThermoFisher).

As used herein, “cation exchange chromatography” (CEX) refers to a form of ion exchange chromatography performed on a solid phase medium (e.g., resin or membrane) that is negatively charged and has the capacity to exchange free cations with cations in an aqueous solution passed over or through the solid phase. The charge may be provided by attaching one or more charged ligands to the solid phase. e.g., via covalent linkage. Alternatively or additionally, the charge may be an inherent property of the solid phase (e.g., silica, which has an overall negative charge). CEX chromatography is typically used to remove high molecular weight (HMW) contaminants, process related impurities, and/or viral contaminants. Commercially available cation exchange media include, but are not limited to, sulphopropyl (SP) immobilized on agarose (e.g., SPSEPHAROSE FAST FLOW™, SP-SEPHAROSE FAST FLOW XL™ or SP-SEPHAROSE HIGH PERFORMANCE™ CAPTO S™, CAPTO SP ImpRes™, CAPTO S ImpAct™ (Cytiva), FRACTOGEL-SO₃™, FRACTOGEL-SE HICAP™, and FRACTOPREP™ (EMD Merck, Darmstadt, Germany), TOYOPEARL® XS, TOYOPEARL® HS (Tosoh Bioscience, King of Prussia, PA), UNOsphere™ (BioRad, Hercules, CA), S Ceramic Hyper™ DF (Pall, Port Washington, NY), POROS™ (ThermoFisher, Waltham, MA), ESHMUNO® CSP and ESHMUNO® CP-FT (Millipore Sigma, Darmstadt, Germany).

As used herein, “hydrophobic interaction chromatography” (HIC) refers to chromatography performed on a solid phase medium that makes use of the interaction between hydrophobic ligands and hydrophobic residues on the surface of a desired solute (e.g., a desired protein). Commercially available hydrophobic interaction chromatography media include, but are not limited to, Phenyl Sephrose™ (Cytiva), Tosoh Hexyl (Tosoh Bioscience), and Capto™ Phenyl (Cytiva).

As used herein, “mixed-mode or multi-modal chromatography” (MMC) refers to chromatography that makes use of more than one form of interaction between the stationary phase and analyte to achieve separation. MMC differs from single mode chromatography in that two or more types of interactions, such as, e.g., electrostatic, hydrogen bonding, and/or hydrophobic interactions, contribute significantly to the retention of solutes. Commercially available multi-modal chromatography media include, but are not limited to, Capto™ Adhere, Capto™ MMC Impress, Capto™ MMC, (Cytiva), PPA Hypercel, MEP Hypercell, HEA Hypercell (Pall Corporation, Port Washington, NY). Eshmuno HCX, (Merk Millipore), and TOYOPEARL® MX-Trp-650M (Tosoh Bioscience).

As used herein, a “production” cell culture medium refers to a cell culture medium that is typically used in a cell culture during the transition when exponential growth is ending and protein production takes over (i.e., “transition” and/or “product” phases) and is sufficiently complete to maintain a desired cell density, viability, and/or product titer during this phase. A production cell culture medium may be the same as or different than the cell culture medium used during the exponential growth phase of the cell culture.

As used herein, a “production phase” of a cell culture refers to the period of time during which logarithmic cell growth has ended and recombinant protein production is predominant.

As used herein, the term “recombinant protein” refers to a heterologous protein produced by a host cell transfected with a nucleic acid encoding the protein when the host cell is cultivated in cell culture.

As used herein, the term “purified,” when used in relation to a composition, refers to a composition wherein at least one impurity is present at a lower concentration in the purified composition relative to the composition as it existed prior to one or more unit operations. Additionally, a “purified” protein (e.g., a purified antibody) refers to a protein which has been increased in purity, such that it exists in a form that is more pure than it exists in its natural environment and/or when initially synthesized and/or amplified under laboratory conditions. Purity is a relative term and does not necessarily refer to absolute purity.

As used herein, “step yield” refers to the amount of product in a pool divided by the amount of product loaded. The amount of product in a pool is calculated as the “(pool volume)×(pool concentration)” and may be determined via UV measurements. The amount of product loaded is calculated as the “(amount loaded)×(load concentration)” and may be determined via UV measurements. For determination of both the pool and the load concentrations, it may be assumed that the predominant species that contributes to the UV signature is the product of interest or a derivative thereof, e.g., HMW impurities/aggregates have similar extinction coefficients and other impurities either do not contribute to the UV signature or are present at small enough amounts for their contribution to the UV signature to be negligible.

As used herein, the term “titrant” refers to a solution of known concentration that is added to another solution during a titration. An “acid titrant” refers to a titrant with a pH of less than 7, while a “base titrant” refers to a titrant with a pH of greater than 7.

As used herein, the term “unit operation” refers to a functional step that is performed as part of a process of purifying a protein of interest. Unit operations can be designed to achieve a single objective or multiple objectives, such as capture, acid precipitation, centrifugation, or chromatography steps. Unit operations can also include holding or storing steps between processing steps.

As used herein, the term “antibody” generally refers to a tetrameric immunoglobulin protein comprising two light chain polypeptides (about 25 kDa each) and two heavy chain polypeptides (about 50-70 kDa each). The term “light chain” or “immunoglobulin light chain” refers to a polypeptide comprising, from amino terminus to carboxyl terminus, a single immunoglobulin light chain variable region (VL) and a single immunoglobulin light chain constant domain (CL). The immunoglobulin light chain constant domain (CL) can be a human kappa (κ) or human lambda (λ) constant domain. The term “heavy chain” or “immunoglobulin heavy chain” refers to a polypeptide comprising, from amino terminus to carboxyl terminus, a single immunoglobulin heavy chain variable region (VH), an immunoglobulin heavy chain constant domain 1 (CH1), an immunoglobulin hinge region, an immunoglobulin heavy chain constant domain 2 (CH2), an immunoglobulin heavy chain constant domain 3 (CH3), and optionally an immunoglobulin heavy chain constant domain 4 (CH4). Heavy chains are classified as mu (μ), delta (Δ), gamma (γ), alpha (α), and epsilon (ε), and define the antibody's isotype as IgM, IgD, IgG, IgA, and IgE, respectively. The IgG-class and IgA-class antibodies are further divided into subclasses, namely, IgG1, IgG2, IgG3, and IgG4, and IgA1 and IgA2, respectively. The heavy chains in IgG, IgA, and IgD antibodies have three constant domains (CH1, CH2, and CH3), whereas the heavy chains in IgM and IgE antibodies have four constant domains (CH1, CH2, CH3, and CH4). The immunoglobulin heavy chain constant domains can be from any immunoglobulin isotype, including subtypes. The antibody chains are linked together via inter-polypeptide disulfide bonds between the CL domain and the CH1 domain (i.e., between the light and heavy chain) and between the hinge regions of the two antibody heavy chains.

Variable regions of immunoglobulin chains generally exhibit the same overall structure, comprising relatively conserved framework regions (FR) joined by three hypervariable regions, more often called “complementarity determining regions” or CDRs. The CDRs from the two chains of each heavy chain and light chain pair typically are aligned by the framework regions to form a structure that binds specifically to a specific epitope on the target protein. From N-terminus to C-terminus, naturally-occurring light and heavy chain variable regions both typically conform with the following order of these elements: FR1, CDR1, FR2, CDR2, FR3, CDR3, and FR4. A numbering system has been devised for assigning numbers to amino acids that occupy positions in each of these domains. This numbering system is defined in Kabat Sequences of Proteins of Immunological Interest (1987 and 1991, NIH, Bethesda, MD), or Chothia & Lesk, 1987, J. Mol. Biol. 196:901-917; Chothia et al., 1989, Nature 342:878-883. The CDRs and FRs of a given antibody may be identified using this system. Other numbering systems for the amino acids in immunoglobulin chains include IMGT® (the international ImMunoGencTics information system; Lefranc et al., Dev. Comp. Immunol. 29:185-203; 2005) and AHo (Honegger and Pluckthun, J. Mol. Biol. 309 (3): 657-670; 2001).

As used in the context of this disclosure, an “antigen-binding fragment.” used interchangeably herein with “binding fragment” or “fragment,” is a portion of an antibody that lacks at least some of the amino acids present in a full-length heavy chain and/or light chain, but which is still capable of specifically binding to an antigen. An antigen-binding fragment includes, but is not limited to, a single-chain variable fragment (scFv), a nanobody (e.g., VH domain of heavy chain only antibodies (e.g., camelid heavy chain antibodies); VHH fragment, see Cortez-Retamozo et al., Cancer Research, Vol. 64:2853-57, 2004), a Fab fragment, a Fab′ fragment, a F(ab′) 2 fragment, a Fv fragment, a Fd fragment, and a CDR fragment, and can be derived from any mammalian source, such as human, mouse, rat, rabbit, or camelid. Antigen-binding fragments may compete for binding of a target antigen with an intact antibody, and the fragments may be produced by the modification of intact antibodies (e.g., enzymatic or chemical cleavage) or synthesized de novo using recombinant DNA technologies or peptide synthesis. In some embodiments, the antigen-binding fragment comprises at least one CDR from an antibody that binds to the antigen, for example, the heavy chain CDR3 from an antibody that binds to the antigen. In other embodiments, the antigen-binding fragment comprises all three CDRs from the heavy chain of an antibody that binds to the antigen or all three CDRs from the light chain of an antibody that binds to the antigen. In still other embodiments, the antigen-binding fragment comprises all six CDRs from an antibody that binds to the antigen (three from the heavy chain and three from the light chain).

Papain digestion of antibodies produces two identical antigen-binding fragments, called “Fab” fragments, each with a single antigen-binding site, and a residual “Fc” fragment which contains all but the first domain of the immunoglobulin heavy chain constant region. The Fab fragment contains the variable domains from the light and heavy chains, as well as the constant domain of the light chain and the first constant domain (CH1) of the heavy chain. Thus, a “Fab fragment” is comprised of one immunoglobulin light chain (light chain variable region (VL) and constant region (CL)) and the CH1 domain and variable region (VH) of one immunoglobulin heavy chain. The heavy chain of a Fab molecule cannot form a disulfide bond with another heavy chain molecule. The “Fd fragment” comprises the VH and CH1 domains from an immunoglobulin heavy chain. The Fd fragment represents the heavy chain component of the Fab fragment.

The “Fc fragment” or “Fc domain” of an immunoglobulin generally comprises two constant domains, a CH2 domain and a CH3 domain, and optionally comprises a CH4 domain. The Fc domain may be an Fc domain from an IgG1, IgG2, IgG3, or IgG4 immunoglobulin. In some embodiments, the Fc domain comprises CH2 and CH3 domains from a human IgG1 or human IgG2 immunoglobulin. The Fc domain may retain effector function, such as C1q binding, complement dependent cytotoxicity (CDC), Fc receptor binding, antibody-dependent cell-mediated cytotoxicity (ADCC), and phagocytosis. In other embodiments, the Fc domain may be modified to reduce or eliminate effector function.

A “Fab′ fragment” is a Fab fragment having at the C-terminus of the CH1 domain one or more cysteine residues from the antibody hinge region.

A “F(ab′)₂ fragment” is a bivalent fragment including two Fab′ fragments linked by a disulfide bridge between the heavy chains at the hinge region.

The “Fv” fragment is the minimum fragment that contains a complete antigen recognition and binding site from an antibody. This fragment consists of a dimer of one immunoglobulin heavy chain variable region (VH) and one immunoglobulin light chain variable region (VL) in tight, non-covalent association. It is in this configuration that the three CDRs of each variable region interact to define an antigen binding site on the surface of the VH-VL dimer. A single light chain or heavy chain variable region (or half of an Fv fragment comprising only three CDRs specific for an antigen) has the ability to recognize and bind antigen, although at a lower affinity than the entire binding site comprising both VH and VL.

A “single-chain variable fragment” or “scFv fragment” comprises the VH and VL regions of an antibody, wherein these regions are present in a single polypeptide chain, and optionally comprising a peptide linker between the VH and VL regions that enables the Fv to form the desired structure for antigen binding (see e.g., Bird et al., Science, Vol. 242:423-426, 1988; and Huston et al., Proc. Natl. Acad. Sci. USA, Vol. 85:5879-5883, 1988).

A “nanobody” is the heavy chain variable region of a heavy-chain antibody. Such variable domains are the smallest fully functional antigen-binding fragment of such heavy-chain antibodies, with a molecular mass of only 15 kDa. See Cortez-Retamozo et al., Cancer Research 64:2853-57, 2004. Functional heavy-chain antibodies devoid of light chains are naturally occurring in certain species of animals, such as nurse sharks, wobbegong sharks, and Camelidae, such as camels, dromedaries, alpacas and llamas. The antigen-binding site is reduced to a single domain, the VHH domain, in these animals. These antibodies form antigen-binding regions using only heavy chain variable region, i.e., these functional antibodies are homodimers of heavy chains only (referred to as “heavy-chain antibodies” or “HCAbs”). Camelized VHH reportedly recombines with IgG2 and IgG3 constant regions that contain hinge, CH2, and CH3 domains and lack a CH1 domain. Camelized VHH domains have been found to bind to antigen with high affinity (Desmyter et al., J. Biol. Chem., Vol. 276:26285-90, 2001) and possess high stability in solution (Ewert et al., Biochemistry, Vol. 41:3628-36, 2002). Methods for generating antibodies having camelized heavy chains are described in, for example, U.S. Patent Publication Nos. 2005/0136049 and 2005/0037421. Alternative scaffolds can be made from human variable-like domains that more closely match the shark V-NAR scaffold and may provide a framework for a long penetrating loop structure. Human heavy-chain antibodies can be produced by transgenic animals expressing human immunoglobulin genes, such as UniAb™ antibodies produced by UniRat™ transgenic rats.

### Chromatography Medium

The disclosed methods utilize chromatography medium, including chromatography medium suitable for overload or frontal chromatography. Chromatography medium that may be used in methods of the present disclosure include, but are not limited to, ion exchange chromatography (IEX) medium, including cation exchange chromatography (CEX) medium and anion exchange chromatography (AEX) medium, multimodal or mixed-mode chromatography (MMC) medium, hydrophobic interaction chromatography (HIC) medium, and hydroxyapatite (HA) medium.

In some embodiments, the chromatography medium is a cation exchange chromatography medium. In some embodiments, the chromatography medium is a low ligand density cation exchange chromatography medium. In some embodiments, the chromatography medium is a cation exchange chromatography medium for frontal chromatography. In some embodiments, the chromatography medium is a cation exchange chromatography column.

In some embodiments, the chromatography medium is a cation exchange chromatography medium with an ionic density of less than 60 μeq/mL, less than 55 μeq/mL, less than 50 μeq/mL, less than 45 μeq/mL, or less than 40 μeq/mL. In some embodiments, the chromatography medium is a cation exchange chromatography medium with an ionic density of less than 55 μeq/mL, less than 50 μeq/mL, less than 45 μeq/mL, or less than 40 μeq/mL. In some embodiments, the chromatography medium is a cation exchange chromatography medium with an ionic density less than 50 μeq/mL, less than 45 μeq/mL, or less than 40 μeq/mL. In some embodiments, the chromatography medium is a cation exchange chromatography medium with an ionic density of less than 45 μeq/mL or less than 40 μeq/mL. In some embodiments, the chromatography medium is a cation exchange chromatography medium with an ionic density of less than 40 μeq/mL. Ionic density may be measured according to methods described in Stone, Matthew T., Kristen A. Cotoni, and Jayson L. Stoner. “Cation exchange frontal chromatography for the removal of monoclonal antibody aggregates.” Journal of Chromatography A 1599 (2019): 152-160.

In some embodiments, the chromatography medium is an anion exchange chromatography medium. In some embodiments, the chromatography medium is an anion exchange chromatography medium for frontal chromatography. In some embodiments, the chromatography medium is an anion exchange chromatography column.

In some embodiments, the chromatography medium is a MMC medium. In some embodiments, the chromatography medium is a MMC medium for frontal chromatography. In some embodiments, the chromatography medium is a MMC column.

In some embodiments, the chromatography medium is a HIC medium. In some embodiments, the chromatography medium is a HIC medium for frontal chromatography. In some embodiments, the chromatography medium is a HIC column.

In some embodiments, the chromatography medium is packed in a chromatography column.

In some embodiments, the chromatography medium is a membrane, a monolith, or a resin. In some embodiments, the chromatography medium is a membrane. In some embodiments, the chromatography medium is a monolith. In some embodiments, the chromatography medium is a resin.

In some embodiments, the chromatography medium has a bed diameter of 50 cm, 60 cm, 70 cm, 80 cm, 90 cm, 100 cm, 110 cm, 120 cm, 130 cm, 140 cm, 150 cm, 160 cm, 170 cm, 180 cm, 190 cm, or 200 cm.

In some embodiments, the bed diameter for the chromatography medium is determined based on the amount of product and/or contaminants in the load.

The bed height of a chromatography medium may be varied to modulate medium volume and achieve a target loading density based on incoming product mass, which may be measured directly or estimated based on historical titers. Varying the bed height may be a more feasible means for modulating the medium volume and consequent loading density than varying the bed diameter because manufacturing sites typically have limited options for changing hardware specifications, equipment availability, and plant layouts. Unlike changes to the column diameter, varying the bed height does not require modifications to equipment or alterations to a plant's layout and/or footprint.

In some embodiments, the chromatography medium has a bed height of at least 5 cm. In some embodiments, the chromatography medium has a bed height in the range of 5 cm to 30 cm. In some embodiments, the chromatography medium has a bed height in the range of 5 cm to 15 cm.

In some embodiments, the chromatography medium has a bed height of 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, 20 cm, 21 cm, 22 cm, 23 cm, 24 cm, 25 cm, 26 cm, 27 cm, 28 cm, 29 cm, or 30 cm. In some embodiments, the chromatography medium has a bed height of 10 cm, 11 cm, 12 cm, 13 cm, 14 cm, or 15 cm.

In some embodiments, the bed height for the chromatography medium is determined based on the amount of product and/or contaminants in the load. In some embodiments, the bed height for the chromatography medium is determined based on a target residence time. In some embodiments, the bed height for the chromatography medium is determined based on a target residence time, wherein the flow rate is a constant value in the range of 50 cm/hr to 250 cm/hr (such as, e.g., 50 cm/hr, 75 cm/hr, 100 cm/hr, 125 cm/hr, 150 cm/hr, 175 cm/hr, 200 cm/hr. 225 cm/hr, 250 cm/hr).

In some embodiments, the chromatography medium is a column with a volume of at least 1 mL, at least 2 mL, at least 3 mL, at least 4 mL, at least 5 mL, at least 6 mL, at least 7 mL, at least 8 mL, at least 9 mL, at least 10 mL, at least 15 mL, at least 20 mL, at least 25 mL, at least 30 mL, at least 40 mL, at least 50 mL, at least 75 mL, at least 100 mL, at least 200 mL, at least 300 mL, at least 400 mL, at least 500 mL, at least 600 mL, at least 700 mL, at least 800 mL, at least 900 mL, at least 1 L, at least 2 L, at least 3 L, at least 4 L, at least 5 L, at least 6 L, at least 7 L, at least 8 L, at least 9 L, at least 10 L, at least 25 L, at least 50 L, at least 100 L, at least 200 L, at least 300 L, at least 400 L, at least 500 L, at least 600 L, at least 700 L, at least 800 L, at least 900 L or at least 1000 L.

### Equilibration Solution

The chromatography medium may be equilibrated with an equilibration solution prior to the loading. Selection of an appropriate equilibration solution and/or equilibration solution concentration is within the ability of one ordinarily skilled in the art. In some embodiments, the equilibration solution comprises 50 mM acetate and has a pH value of 5.0.

In some embodiments, the equilibration solution comprises acetate. In some embodiments, the equilibration solution comprises acetate at a concentration in the range of 10 mM to 100 mM, 20 mM to 100 mM, 30 mM to 100 mM, 40 mM to 100 mM, 10 mM to 90 mM, 10 mM to 80 mM, 10 mM to 70 mM. 10 mM to 60 mM, 20 mM to 60 mM, or 30 mM to 60 mM.

In some embodiments, the equilibration solution comprises acetate at a concentration of 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM.

In some embodiments, the pH of the equilibration solution is in the range of 4.0 to 8.0, 5.0 to 8.0, 6.0 to 8.0, 7.0 to 8.0, 4.0 to 7.0, 4.0 to 6.0, 4.0 to 5.0, or 5.0 to 6.0. In some embodiments, the pH of the equilibration solution is 4.0, 4.5, 5.0, 5.5, 6.0, 6.5, 7.0, 7.5, or 8.0. In some embodiments, the pH of the equilibration solution is 5.0.

In some embodiments, 3 to 10, 3 to 9, 3 to 8, 3 to 7, 3 to 6, 3 to 5, 3 to 4, 4 to 10, 4 to 9, 4 to 8, 4 to 7, 4 to 6, 4 to 5, 5 to 10, 6 to 10, 6 to 9, 6 to 8, 6 to 7, 7 to 10, 7 to 9, 7 to 8, 8 to 10, 8 to 9, or 9 to 10 medium volumes (e.g., column volumes) of equilibration solution are passed through the chromatography medium prior to loading the composition. In some embodiments, 3, 4, 5, 6, 7, 8, 9, or 10 medium volumes (e.g., column volumes) of equilibration solution are passed through the chromatography medium prior to loading the composition.

### Load Solution

Some embodiments of the present disclosure involve loading a composition, such as a load solution, comprising a protein and one or more impurities onto a chromatography medium. The load solution may be derived from an effluent stream and/or product pool originating from a previous upstream unit operation, such as a cell culture harvest, capture chromatography, affinity chromatography, polishing chromatography, viral inactivation, neutralization, depth filtration, virus filtration, UF/DF, or other operation. Prior to loading, load feed dilution or conditioning may be performed to achieve a target pH, conductivity, buffer composition, and the like for the load solution. In some embodiments, the load solution is derived from an effluent stream or product pool from a high capacity chromatography medium capable of processing high column loading. Non-limiting examples of high capacity chromatography media include MabSelect™ PrismA™, Capto™ Q (Cytiva, Marlborough, MA), PRAESTO® Jetted resins (Purolite, King of Prussia, PA), Porous™ XQ (Waltham, MA), Eshmuno™ MQ (EMD Millipore, Burlington, MA), TOYOPEARL® GigaCap, and TOYOPEARL® NH2-750F (Tosoh Bioscience, Tokyo, Japan).

The concentration of the product of interest in the load solution may be determined by measuring UV absorbance at 280 nm and/or A300 to determine the load volume for each cycle of a frontal loading chromatography step. In some embodiments, the concentration of the product of interest is suitable to meet production scheduling and/or timelines. In some embodiments, the concentration of the product of interest in the load solution is greater than or equal to 5 g/L.

Selection of an appropriate load solution and/or load solution concentration is within the ability of one ordinarily skilled in the art to which this disclosure pertains. Such load solutions may comprise, but are not limited to comprising, acetate, citrate, phosphate, glycine, L-arginine, L-histidine, and 2-(N-morpholinojethanesulfonic acid (MES). In some embodiments, the load solution and/or load solution concentration is the same as the effluent stream and/or pool containing the product of interest from a previous upstream operation.

In some embodiments, the load solution comprises acetate. In some embodiments, the load solution comprises acetate at a concentration in the range of 25 mM to 200 mM. In some embodiments, the load solution comprises acetate at a concentration in the range of 50 mM to 100 mM. In some embodiments, the load solution comprises acetate at a concentration of at least 25 mM. In some embodiments, the load solution comprises acetate at a concentration of at least 30 mM. In some embodiments, the load solution comprises acetate at a concentration of at least 35 mM. In some embodiments, the load solution comprises acetate at a concentration of at least 40 mM. In some embodiments, the load solution comprises acetate at a concentration of at least 45 mM. In some embodiments, the load solution comprises acetate at a concentration of at least 50 mM. In some embodiments, the load solution comprises acetate at a concentration of at least 55 mM. In some embodiments, the load solution comprises acetate at a concentration of at least 60 mM. In some embodiments, the load solution comprises acetate at a concentration of at least 65 mM. In some embodiments, the load solution comprises acetate at a concentration of at least 70 mM. In some embodiments, the load solution comprises acetate at a concentration of at least 75 mM. In some embodiments, the load solution comprises acetate at a concentration of at least 80 mM. In some embodiments, the load solution comprises acetate at a concentration of at least 85 mM. In some embodiments, the load solution comprises acetate at a concentration of at least 90 mM. In some embodiments, the load solution comprises acetate at a concentration of at least 95 mM. In some embodiments, the load solution comprises acetate at a concentration of at least 100 mM. In some embodiments, the load solution comprises acetate at a concentration of at least 125 mM. In some embodiments, the load solution comprises acetate at a concentration of at least 150 mM. In some embodiments, the load solution comprises acetate at a concentration of at least 175 mM. In some embodiments, the load solution comprises acetate at a concentration of at least 200 mM.

In some embodiments, the load solution comprises acetate at a concentration of 50 mM, 51 mM, 52 mM, 53 mM, 54 mM, 55 mM, 56 mM, 57 mM, 58 mM, 59 mM, 60 mM, 65 mM, 70 mM, 75 mM, 80 mM, 85 mM, 90 mM, 95 mM, or 100 mM.

In some embodiments, the load solution comprises acetate at a concentration of 50 mM. In some embodiments, the load solution comprises acetate at a concentration of 51 mM. In some embodiments, the load solution comprises acetate at a concentration of 52 mM. In some embodiments, the load solution comprises acetate at a concentration of 53 mM. In some embodiments, the load solution comprises acetate at a concentration of 54 mM. In some embodiments, the load solution comprises acetate at a concentration of 55 mM. In some embodiments, the load solution comprises acetate at a concentration of 56 mM. In some embodiments, the load solution comprises acetate at a concentration of 57 mM. In some embodiments, the load solution comprises acetate at a concentration of 58 mM. In some embodiments, the load solution comprises acetate at a concentration of 59 mM. In some embodiments, the load solution comprises acetate at a concentration of 60 mM. In some embodiments, the load solution comprises acetate at a concentration of 65 mM. In some embodiments, the load solution comprises acetate at a concentration of 70 mM. In some embodiments, the load solution comprises acetate at a concentration of 75 mM. In some embodiments, the load solution comprises acetate at a concentration of 80 mM. In some embodiments, the load solution comprises acetate at a concentration of 85 mM. In some embodiments, the load solution comprises acetate at a concentration of 90 mM. In some embodiments, the load solution comprises acetate at a concentration of 95 mM. In some embodiments, the load solution comprises acetate at a concentration of 100 mM.

The pH of the load solution can impact binding and impurity clearance by modulating the affinity of the load components for the chromatography medium. In some embodiments, the pH of the load solution is in the range of 3.6. to 6.0. In some embodiments, the pH is in the range of 4.0 to 5.6. In some embodiments, the pH is in the range of 4.5 to 5.6. In some embodiments, the pH is in the range of 5.0 to 5.6. In some embodiments, the pH is in the range of 4.5 to 5.5. In some embodiments, the pH is at least 4.0. In some embodiments, the pH is at least 4.1. In some embodiments, the pH is at least 4.2. In some embodiments, the pH is at least 4.3. In some embodiments, the pH is at least 4.4. In some embodiments, the pH is at least 4.5. In some embodiments, the pH is at least 4.6. In some embodiments, the pH is at least 4.7. In some embodiments, the pH is at least 4.8. In some embodiments, the pH is at least 4.9. In some embodiments, the pH is at least 5.0. In some embodiments, the pH is at least 5.1. In some embodiments, the pH is at least 5.2. In some embodiments, the pH is at least 5.3. In some embodiments, the pH is at least 5.4. In some embodiments, the pH is 5.5. In some embodiments, the pH is 5.6. In some embodiments, the pH is 3.6, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or 6.0. In some embodiments, the pH of the load solution is at least 6.0. In some embodiments, formic acid is used maintain the pH of the load solution.

The conductivity of the load solution may impact binding and impurity clearance. Load feeds with load solutions having low conductivity may promote stronger protein binding during loading. Low conductivity is correlated with less conditioning and higher protein concentration, both of which may improve facility fit (i.e., enabling small overall pool volumes that facilitate a more intensified process). In some embodiments, the conductivity of the load solution is 10 mS/cm or less, such as 9 mS/cm or less, 8 mS/cm or less, 7 mS/cm or less, 6 mS/cm or less, or 5 mS/cm or less.

In some embodiments, the conductivity is in the range of 4 mS/cm to 10 mS/cm, such as, e.g., in the range of 4 mS/cm to 5 mS/cm. In some embodiments, the conductivity is 4.1 mS/cm, 4.2 mS/cm, 4.3 mS/cm, 4.4 mS/cm, 4.5 mS/cm, 4.6 mS/cm, 4.7 mS/cm, 4.8 mS/cm, 4.9 mS/cm, 5.0 mS/cm, 5.1 mS/cm, 5.2 mS/cm, 5.3 mS/cm, 5.4 mS/cm, 5.5 mS/cm, 5.6 mS/cm, 5.7 mS/cm, 5.8 mS/cm, 5.9 mS/cm, 6.0 mS/cm, 6.1 mS/cm, 6.2 mS/cm, 6.3 mS/cm, 6.4 mS/cm, 6.5 mS/cm, 6.6 mS/cm, 6.7 mS/cm, 6.8 mS/cm, 6.9 mS/cm, 7.0 mS/cm, 7.1 mS/cm, 7.2 mS/cm, 7.3 mS/cm, 7.4 mS/cm, 7.5 mS/cm, 7.6 mS/cm, 7.7 mS/cm, 7.8 mS/cm, 7.9 mS/cm, 8.0 mS/cm, 8.1 mS/cm, 8.2 mS/cm, 8.3 mS/cm, 8.4 mS/cm, 8.5 mS/cm, 8.6 mS/cm, 8.7 mS/cm, 8.8 mS/cm, 8.9 mS/cm, 9.0 mS/cm, 9.1 mS/cm, 9.2 mS/cm, 9.3 mS/cm, 9.4 mS/cm, 9.5 mS/cm, 9.6 mS/cm, 9.7 mS/cm, 9.8 mS/cm, 9.9 mS/cm, or 10.0 mS/cm.

In some embodiments, the conductivity is 4.0 mS/cm. In some embodiments, the conductivity is 4.1 mS/cm. In some embodiments, the conductivity is 4.2 mS/cm. In some embodiments, the conductivity is 4.3 mS/cm. In some embodiments, the conductivity is 4.4 mS/cm. In some embodiments, the conductivity is 4.5 mS/cm. In some embodiments, the conductivity is 4.6 mS/cm. In some embodiments, the conductivity is 4.7 mS/cm. In some embodiments, the conductivity is 4.8 mS/cm. In some embodiments, the conductivity is 4.9 mS/cm. In some embodiments, the conductivity is 5.0 mS/cm.

In some embodiments, the conductivity of the load solution may be modulated via the use of a low buffering capacity buffer. In some embodiments, the load solution has a low buffering capacity. In some embodiments, the conductivity of the load solution may be established by the conditions under which the load feed was derived. In some embodiments, an acid capable of establishing and maintaining a low buffering capacity for the load feed may be used. In some embodiments, the acid is capable of establishing and maintaining the low buffering capacity of a load feed originating from a low pH viral inactivation operation. In some embodiments, the acid is formic acid. The combination of a low buffering capacity buffer and formic acid may also be used to minimize the volume of base titrant (e.g., 2M Tris base or a similar base titrant) required to raise the pH to target conditions suitable for use on the chromatography medium (e.g., the cation exchange chromatography medium), thereby minimizing any increase in the conductivity of the load feed. Lowering the conductivity of the load feed may also be achieved by diluting the load feed with water or another suitable buffering solution.

### Loading Density

Frontal chromatography is characterized by significant binding of the product of interest initially (i.e., at low loading densities), followed by the displacement of the bound product of interest by higher-affinity impurities at higher loading densities, causing the displaced product of interest to flow through in the effluent, which is enriched in the product of interest and collected as a product pool. As such, the step yield for a frontal loading chromatography operation may vary non-linearly with respect to loading.

At low loading densities, a significant fraction of the product loaded onto the resin may remain bound and unrecoverable, leading to low yield. Conversely, as the loading density is increased and the resin becomes more saturated with impurities, an increasing fraction of the product will be displaced and flow through, leading to more linear yield increases.

Prior to the start of the chromatography operation, the product concentration of the load solution may be determined by measuring absorbance at 280 nm or 300 nm. The product concentration of the load solution may be used to determine the load volume for each cycle of the frontal chromatography step.

Some embodiments of the present disclosure involve loading a composition, such as a load solution, comprising a protein and one or more impurities onto a chromatography medium. In some embodiments, the loading density is at least 200 g/L-r. In some embodiments, the loading density is 1000 g/L-r or more. In some embodiments, the loading density is in the range of 1000 g/L-r to 1500 g/L-r. In some embodiments, the loading density is in the range of 500 g/L-r to 1,500 g/L-r. In some embodiments, the loading density is in the range of 500 g/L-r to 1.100 g/L-r. In some embodiments, the loading density is in the range of 500 g/L-r to 1.000 g/L-r. In some embodiments, the loading density is in the range of 500 g/L-r to 950 g/L-r. In some embodiments, the loading density is in the range of 500 g/L-r to 900 g/L-r. In some embodiments, the loading density is in the range of 500 g/L-r to 850 g/L-r. In some embodiments, the loading density is in the range of 500 g/L to 800 g/L-r. In some embodiments, the loading density is in the range of 500 g/L-r to 750 g/L-r. In some embodiments, the loading density is in the range of 500 g/L-r to 700 g/L-r. In some embodiments, the loading density is in the range of 500 g/L-r to 650 g/L-r. In some embodiments, the loading density is in the range of 500 g/L-r to 600 g/L-r. In some embodiments, the loading density is in the range of 500 g/L-r to 550 g/L-r. In some embodiments, the loading density is in the range of 700 g/L-r to 1.100 g/L-r. In some embodiments, the loading density is in the range of 700 g/L-r to 1.000 g/L-r. In some embodiments, the loading density is in the range of 700 g/L-r to 950 g/L-r. In some embodiments, the loading density is in the range of 700 g/L-r to 900 g/L-r. In some embodiments, the loading density is in the range of 700 g/L-r to 850 g/L-r. In some embodiments, the loading density is in the range of 700 g/L-r to 800 g/L-r. In some embodiments, the loading density is in the range of 700 g/L-r to 750 g/L-r.

In some embodiments, the loading density is at least 200, at least 300, at least 400, at least 500, at least 525, at least 550, at least 575, at least 600, at least 625, at least 650, at least 675, at least 700, at least 725, at least 750, at least 775, at least 800, at least 825, at least 850, at least 875, at least 900, at least 925, at least 950, at least 975, at least 1,000, at least 1,100, at least 1,200, at least 1,300, at least 1,400, at least 1,500, at least 2,000, or at least 3,000 g/L-r. In some embodiments, the loading density is at least 500 g/L-r. In some embodiments, the loading density is at least 525 g/L-r. In some embodiments, the loading density is at least 550 g/L-r. In some embodiments, the loading density is at least 575 g/L-r. In some embodiments, the loading density is at least 600 g/L-r. In some embodiments, the loading density is at least 625 g/L-r. In some embodiments, the loading density is at least 650 g/L-r. In some embodiments, the loading density is at least 675 g/L-r. In some embodiments, the loading density is at least 700 g/L-r. In some embodiments, the loading density is at least 725 g/L-r. In some embodiments, the loading density is at least 750 g/L-r. In some embodiments, the loading density is at least 775 g/L-r. In some embodiments, the loading density is at least 800 g/L-r. In some embodiments, the loading density is at least 825 g/L-r. In some embodiments, the loading density is at least 850 g/L-r. In some embodiments, the loading density is at least 875 g/L-r. In some embodiments, the loading density is at least 900 g/L-r. In some embodiments, the loading density is at least 925 g/L-r. In some embodiments, the loading density is at least 950 g/L-r. In some embodiments, the loading density is at least 975 g/L-r. In some embodiments, the loading density is at least 1.000 g/L-r. In some embodiments, the loading density is at least 1,100 g/L-r. In some embodiments, the loading density is at least 1.200 g/L-r. In some embodiments, the loading density is at least 1,300 g/L-r. In some embodiments, the loading density is at least 1,400 g/L-r. In some embodiments, the loading density is at least 1,500 g/L-r. In some embodiments, the loading density is at least 500, at least 600, at least 700, at least 800, at least 900, at least 1,000, at least 1,100, at least 1,500, at least 2,000, or at least 3,000 g/L-r.

In some embodiments, the loading density is 200, 300, 400, 500, 525, 550, 575, 600, 625, 650, 675, 700, 725, 750, 775, 800, 825, 850, 875, 900, 925, 950, 975, 1,000, 1,100, 1,200, 1,300, 1,400, 1,500, 2,000, or 3,000 g/L-r. In some embodiments, the loading density is 500, 600, 700, 800, 900, 1,000, 1,100, 1,500, 2,000, or 3,000 g/L-r.

In some embodiments, the loading density is sufficient to achieve a step yield in the range of 60% to 100%. In some embodiments, the loading density is sufficient to achieve a step yield of at least 70%. In some embodiments, the loading density is sufficient to achieve a step yield of at least 80%. In some embodiments, the loading density is sufficient to achieve a step yield of at least 90%. In some embodiments, the loading density is sufficient to achieve a step yield of at least 92.5%. In some embodiments, the loading density is sufficient to achieve a step yield of at least 95%. In some embodiments, the loading density is sufficient to achieve a step yield of at least 97.5%.

In some embodiments, the loading density is sufficient to achieve a concentration of high molecular weight species that is at least 0.5% lower in the product pool compared to the load feed. In some embodiments, the concentration of HMW species in the product pool is in the range of 1% to 10% lower in the product pool compared to the load feed. In some embodiments, the loading density is sufficient to achieve a concentration of high molecular weight species that is at least 1%, at least 1.5%, at least 2%, at least 2.5%, at least 3%, at least 3.5%, at least 4%, at least 4.5%, at least 5%, at least 6%, at least 7%, at least 7.5%, at least 8%, at least 8.5%, at least 9%, or at least 9.5% lower in the product pool compared to the load feed.

In some embodiments, the loading density is sufficient to achieve a concentration of high molecular weight species that is 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%.

In some embodiments, the concentration of HMW species is 1% lower in the product pool compared to the load feed. In some embodiments, the concentration of HMW species is 2% lower in the product pool than in the load feed. In some embodiments, the concentration of HMW species is 3% lower in the product pool than in the load feed. In some embodiments, the concentration of HMW species is 4% lower in the product pool than in the load feed. In some embodiments, the concentration of HMW species is 5% lower in the product pool than in the load feed. In some embodiments, the concentration of HMW species is 6% lower in the product pool than in the load feed. In some embodiments, the concentration of HMW species is 7% lower in the product pool than in the load feed. In some embodiments, the concentration of HMW species is 8% lower in the product pool than in the load feed. In some embodiments, the concentration of HMW species is 9% lower in the product pool than in the load feed. In some embodiments, the concentration of HMW species is 10% lower in the product pool than in the load feed.

In some embodiments, the concentration of HMW species is 1% to 10% lower in the product pool compared to the load feed.

In some embodiments, the loading density is sufficient to achieve a concentration of host cell protein impurities that is at least 1.5× lower in the product pool compared to the load feed. In some embodiments, the loading density is sufficient to achieve a concentration of HCP impurities that is in the range of 2× to 10× lower in the product pool compared to the load feed. In some embodiments, the loading density is sufficient to achieve a concentration of HCP impurities that is at least 2× lower in the product pool compared to the load feed. In some embodiments, the loading density is sufficient to achieve a concentration of HCP impurities that is at least 3× lower in the product pool compared to the load feed. In some embodiments, the loading density is sufficient to achieve a concentration of HCP impurities that is at least 4× lower in the product pool compared to the load feed. In some embodiments, the loading density is sufficient to achieve a concentration of HCP impurities that is at least 5× lower in the product pool compared to the load feed. In some embodiments, the loading density is sufficient to achieve a concentration of HCP impurities that is at least 6× lower in the product pool compared to the load feed. In some embodiments, the loading density is sufficient to achieve a concentration of HCP impurities that is at least 7× lower in the product pool compared to the load feed. In some embodiments, the loading density is sufficient to achieve a concentration of HCP impurities that is at least 8× lower in the product pool compared to the load feed. In some embodiments, the loading density is sufficient to achieve a concentration of HCP impurities that is at least 9× lower in the product pool compared to the load feed. In some embodiments, the loading density is sufficient to achieve a concentration of HCP impurities that is 10× lower in the product pool compared to the load feed.

### Collection of the Product of Interest

The effluent containing the product of interest from the frontal chromatography operation may be diverted for loading onto another unit operation or collected as a product pool. The effluent containing the product of interest may alternatively be collected as a fixed volume. The collection criteria may be any desired end point, such as, e.g., product and/or impurity concentration, volume, time, process, and/or production parameters.

In some embodiments, the effluent containing the product of interest may be collected as individual fractions over time. The fractions containing the product of interest may be kept as separate product pools or combined into a single product pool.

In some embodiments, the effluent is monitored by UV. Selection of appropriate UV absorbance criteria is within the ability of one of ordinary skill in the art. In some embodiments, the effluent is sent to waste until the UV absorbance reaches a fixed absorbance value and collection of the product begins. In some embodiments, the start collection criterion is based on absorbance. In some embodiments, the effluent is monitored at A280 (i.e., absorbance at 280 nm), and product collection starts at a fixed absorbance value. In some embodiments, product collection begins at a fixed absorbance value in the range of 0.4 AU/cm to 0.6 AU/cm at A280. In some embodiments, product collection begins at a fixed absorbance value in the range of 0.4 AU/cm to 0.5 AU/cm at A280. In some embodiments, product collection begins at a fixed absorbance value in the range of 0.5 AU/cm to 0.6 AU/cm at A280. In some embodiments, product collection begins at a fixed absorbance value of at least 0.5 AU/cm at A280. In some embodiments, product collection begins at a fixed absorbance value of 0.5 AU/cm at A280. In some embodiments, product collection begins at a fixed absorbance value of 0.6 AU/cm at A280.

Product quality for each product pool may optionally be determined for each run using methods and equipment known in the art.

As loading progresses, the effluent is enriched in the product of interest as the chromatography medium becomes saturated by higher affinity impurities. Beyond this saturation point, additional impurities loaded onto the chromatography medium are expected to flow through into the effluent along with the product of interest. The saturation point for critical impurities designed to bind during the frontal loading chromatography step (such as, e.g., HMW product-related impurities) typically represents the practical upper limit for column loading. In some embodiments, collection of the effluent stops prior to or at this upper limit.

In some embodiments, the collection of the effluent may be monitored by UV absorbance. Effluent collection may cease when the UV absorbance reaches a fixed percentage of peak maximum at a fixed UV absorbance.

In some embodiments, the UV absorbance is A280 (280 nm). In some embodiments, the stop collection criterion is 40-60% of peak maximum at A280. In some embodiments, the stop collection criterion is 40-50% of peak maximum at A280. In some embodiments, the stop collection criterion is 50-60% of peak maximum at A280. In some embodiments, the stop collection criterion is 40% of peak maximum at A280. In some embodiments, the stop collection criterion is 50% of peak maximum at A280. In some embodiments, the stop collection criterion is 60% of peak maximum at A280.

In some embodiments, the UV absorbance is A300. In some embodiments, the stop collection criterion is 20-50% of peak maximum at A300. In some embodiments, the stop collection criterion is 40-50% of peak maximum at A300. In some embodiments, the stop collection criterion is at least 20% of peak maximum at A300. In some embodiments, the stop collection criterion is at least 30% of peak maximum at A300. In some embodiments, the stop collection criterion is at least 40% of peak maximum at A300. In some embodiments, the stop collection criterion is at least 45% of peak maximum at A 300. In some embodiments, the stop collection criterion is at least 50% of peak maximum at A300.

The stop collection criterion may be based on a fixed optical density (OD). In some embodiments, the fixed OD is in the range of 0.2 to 3.0.

### Post-Load Wash

Once loading is completed, the chromatography medium may be subjected to an optional post-load wash to recover any remaining unbound product. The product-containing effluent from the post-load wash may be collected and stored as a separate product pool or collected and added to the product pool from the load. The wash effluent may be collected as individual fractions over time or as a fixed volume.

Selection of an appropriate post-load wash solution and/or post-load wash solution concentration is within the ability of one ordinarily skilled in the art. In some embodiments, the post-load wash solution is the same as the non-denaturing cleaning solution. In some embodiments, the post-load wash comprises acetate. In some embodiments, the post-load wash comprises acetate at a concentration in the range of 50 nM to 100 mM. In some embodiments, the post-load wash solution comprises a salt. In some embodiments, the post-load wash solution comprises sodium chloride. In some embodiments, the post-load wash solution comprises sodium chloride at a concentration of less than or equal to 20 mM.

In some embodiments, the conductivity of the post-load wash solution is greater than or equal to the conductivity of the load solution. In some embodiments, the conductivity of the post-load wash solution is greater than or equal to 5.0 mS/cm. In some embodiments, the conductivity of the post-load wash solution is in the range of 5.0 mS/cm to 10.0 mS/cm, such as, e.g., in the range of 5.0 mS/cm to 6.0 mS/cm. In some embodiments, the conductivity of the post-load wash solution is 5.0 mS/cm.

In some embodiments, the conductivity of the post-load wash solution is 5.0 mS/cm, 5.1 mS/cm. 5.2 mS/cm, 5.3 mS/cm, 5.4 mS/cm, 5.5 mS/cm, 5.6 mS/cm, 5.7 mS/cm, 5.8 mS/cm, 5.9 mS/cm, 6.0 mS/cm, 6.5 mS/cm, 7.0 mS/cm, 7.5 mS/cm, 8.0 mS/cm, 8.5 mS/cm, 9.0 mS/cm, 9.5 mS/cm, or 10.0 mS/cm. In some embodiments, the conductivity of the post-load wash solution is 5.1 mS/cm. In some embodiments, the conductivity of the post-load wash solution is 5.2 mS/cm. In some embodiments, the conductivity of the post-load wash solution is 5.3 mS/cm. In some embodiments, the conductivity of the post-load wash solution is 5.4 mS/cm. In some embodiments, the conductivity of the post-load wash solution is 5.5 mS/cm. In some embodiments, the conductivity of the post-load wash solution is 5.6 mS/cm. In some embodiments, the conductivity of the post-load wash solution is 5.7 mS/cm. In some embodiments, the conductivity of the post-load wash solution is 5.8 mS/cm. In some embodiments, the conductivity of the post-load wash solution is 5.9 mS/cm. In some embodiments, the conductivity of the post-load wash solution is 6.0 mS/cm. In some embodiments, the conductivity of the post-load wash solution is 6.5 mS/cm. In some embodiments, the conductivity of the post-load wash solution is 7.0 mS/cm. In some embodiments, the conductivity of the post-load wash solution is 7.5 mS/cm. In some embodiments, the conductivity of the post-load wash solution is 8.0 mS/cm. In some embodiments, the conductivity of the post-load wash solution is 8.5 mS/cm. In some embodiments, the conductivity of the post-load wash solution is 9.0 mS/cm. In some embodiments, the conductivity of the post-load wash solution is 9.5 mS/cm. In some embodiments, the conductivity of the post-load wash solution is 10.0 mS/cm.

In some embodiments, the pH of the post-load wash solution is greater than or equal to the pH of the load solution. In some embodiments, the pH of the post-load wash solution is greater than or equal to 5.0. In some embodiments, the pH is in the range of 5.0 to 5.6. In some embodiments, the pH is at least 5.0. In some embodiments, the pH is at least 5.1. In some embodiments, the pH is at least 5.2. In some embodiments, the pH is at least 5.3. In some embodiments, the pH is at least 5.4. In some embodiments, the pH is 5.5. In some embodiments, the pH is at least 5.6.

In some embodiments, the pH is 5.0, 5.1, 5.2, 5.3, 5.4, 5.5, or 5.6. In some embodiments, the pH is 5.0. In some embodiments, the pH is 5.1. In some embodiments, the pH is 5.2. In some embodiments, the pH is 5.3. In some embodiments, the pH is 5.4. In some embodiments, the pH is 5.5. In some embodiments, the pH is 5.6.

In some embodiments, the volume collected from the post-load wash is less than or equal to 1.0 medium volume (MV) of wash solution to limit impurities from entering the final product pool. In some embodiments, 0.1 MV to 1.0 MV of wash solution is applied to the column. In some embodiments, 0.2 MV of wash solution is applied to the column. In some embodiments, 0.3 MV of wash solution is applied to the column. In some embodiments, 0.4 MV of wash solution is applied to the column. In some embodiments, 0.5 MV of wash solution is applied to the column. In some embodiments, 0.6 MV of wash solution is applied to the column. In some embodiments, 0.7 MV of wash solution is applied to the column. In some embodiments, 0.8 MV of wash solution is applied to the column. In some embodiments, 0.9 MV of wash solution is applied to the column. In some embodiments, 1.0 MV of wash solution is applied to the column.

In some embodiments, the stop criteria for collecting product-containing effluent from the post-load wash is achieved when UV absorbance reaches a fixed percentage of the maximum observed during product loading. In some embodiments, the stop collection criterion is in the range of 75% to 85% of peak maximum at A300. In some embodiments, the stop collection criterion is in the range of 80% to 85% of peak maximum at A300. In some embodiments, the stop collection criterion is at least 75% of peak maximum at A300. In some embodiments, the stop collection criterion is at least 80% of peak maximum at A300. In some embodiments, the stop collection criterion is at least 85% of peak maximum at A300. In some embodiments, the stop collection criterion is 75%, 76%, 77%, 78%, 79%, 80%, 81%, 82%, 83%, 84%, or 85% of peak maximum at A300.

In some embodiments, the stop collection criterion is in the range of 40% to 60% of peak maximum at A300. In some embodiments, the stop collection criterion is in the range of 50% to 60% of peak maximum at A300. In some embodiments, the stop collection criterion is at least 40% of peak maximum at A300. In some embodiments, the stop collection criterion is at least 45% of peak maximum at A300. In some embodiments, the stop collection criterion is at least 50% of peak maximum at A300. In some embodiments, the stop collection criterion is at least 55% of peak maximum at A300. In some embodiments, the stop collection criterion is at least 60% of peak maximum at A300. In some embodiments, the stop collection criterion is 40%, 41%, 42%, 43%, 44%, 45%, 46%, 47%, 48%, 49%, 50%, 51%, 52%, 53%, 54%, 55%, 56%, 57%, 58%, 59%, or 60% of peak maximum at A300.

Alternatively, the stop collection criterion may be based on a fixed optical density (OD), such as, e.g., an OD in the range of 0.2 to 3.0.

### Cleaning the Chromatography Medium

Cleaning may prolong the functional lifetime of the chromatography medium, allowing its reuse over many cycles. Cleaning of a chromatography medium may consist of one or more non-denaturing cleaning steps and/or one or more denaturing regeneration cleaning steps. Non-denaturing cleaning steps strip bound impurities from the chromatography medium, while denaturing cleaning step remove additional bound impurities and sanitize the chromatography medium for reuse or storage.

Linear salt gradient cleaning methods described herein are useful for the non-denaturing cleaning of chromatography media that are highly saturated with product-related impurities (e.g., chromatography media operated in frontal chromatography mode with high loading densities; chromatography media operated in a mode in which non-linear yield vs. loading density is observed). In some embodiments, the chromatography medium is highly saturated with impurities at the end of a cycle of chromatography operation. In some embodiments, the chromatography medium was subjected to a high loading density during chromatography operation.

In some embodiments, the linear salt gradient slowly desorbs and elutes bound impurities relative to an isocratic cleaning step. During isocratic cleaning, bound impurities may desorb rapidly, potentially increasing column pressure beyond hardware capabilities. By contrast, linear salt gradient cleaning may stabilize operating pressures and reduce fouling of the chromatography medium. The linear salt gradient cleaning described herein can be used alone as a non-denaturing cleaning step or in combination with an isocratic denaturing cleaning step to further remove bound impurities and/or contaminants from a chromatography medium for reuse.

In some embodiments, the bound impurities comprise process-related impurities, which typically originate within the manufacturing process and include, but are not limited to, cell substrates such as host cell proteins, nucleic acids (e.g., chromosomal or extrachromosomal DNA, t-RNA, rRNA, or mRNA), lipids (e.g., cell wall material), cell culture components (e.g., media components, serum, inducers, antibiotics, surfactants, antifoam waste products), chromatographic media used in other purification operations (e.g., Protein A ligands), solvents, buffer components, adventitious agents such as endotoxins and viruses, or combinations thereof, as well as extractables and/or leachable (e.g., beta glucans in depth filters). In some embodiments, the bound impurities may also comprise product-related impurities, such as variants of the product of interest that do not share the same properties with respect to activity, efficacy, and/or safety as the product of interest and as such, their presence in the purified drug substance or the final drug product can have a negative impact on product quality, safety, and/or efficacy. Product-related impurities include high molecular weight (HMW) and low molecular weight (LMW) species. HMW species include, among others, dimers (e.g., homodimers), oligomers, and higher order aggregates. LMW species tend to have a less positive charge or a similar surface charge relative to the intact product of interest and elute with or before the product of interest. LMW species include, among others, truncated proteins expressed during culture, degraded proteins, and enzymatically clipped proteins. These impurities may result from structural heterogeneity that occurs during expression, e.g., as a result of process conditions, such as pH and/or shear. In some embodiments, the bound impurities comprise viruses and microorganisms, such as bacteria and fungi. Such bioburden must be reduced or removed during downstream processing as its presence in the final drug substance or drug product will render the material unusable.

Frequent cleaning helps control the accumulation of impurities and contaminants that could be carried over from one cycle to the next and possibly end up in the product stream. A chromatography medium can be cleaned following every chromatography batch in a manufacturing process. In some embodiments, at least one linear salt gradient non-denaturing cleaning step is performed following one or more cycles of the chromatography column. Cleaning with at least a linear salt gradient as described herein following a cycle may reduce fouling, minimize deterioration of the packed column, allow for recycling of the chromatography medium, and/or prolong the lifespan of the chromatography medium. At least one linear salt gradient non-denaturing cleaning step in combination with one or more denaturing cleaning steps may be performed together or separately after one or more cycles, after one or more batches, and/or prior to storage. In some embodiments, a linear salt gradient cleaning step is performed after each cycle.

### Linear Salt Gradient Buffers

Some embodiments of the present disclosure make use of a linear salt gradient as a non-denaturing cleaning step. Specifically, the salt gradient increases linearly from a low or no salt concentration to a higher salt concentration. In some embodiments, the linear salt gradient is generated using at least two buffers (such as, e.g., 2, 3, 4, 5, 6, 7, 8, 9, or 10 buffers). In some embodiments, the linear salt gradient is generated using two buffers. In some embodiments, the linear salt gradient comprises an increase in salt concentration from 0 mM to 2 M, or 0 mM to 1.9 M, or 0 mM to 1.8 M, or 0 mM to 1.7 M, or 0 mM to 1.6 M, or 0 mM to 1.5 M, or 0 mM to 1.4 M, or 0 mM to 1.3 M, or 0 mM to 1.2 M, or 0 mM to 1.1 M, or 0 mM to 1 M, or 0 mM to 950 mM, or 0 mM to 900 mM, or 0 mM to 850 mM, or 0 mM to 800 mM, or 0 mM to 750 mM, or 0 mM to 700 mM, or 0 mM to 650 mM, or 0 mM to 600 mM, or 0 mM to 550 mM, or 0 mM to 500 mM, or 0 mM to 450 mM, or 0 mM to 400 mM, or 0 mM to 350 mM, or 0 mM to 300 mM, or 0 mM to 250 mM, or 0 mM to 200 mM. In some embodiments, 0 mM in the any of the preceding ranges may be replaced by 5 mM. 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM.

In some embodiments, the linear salt gradient comprises an increase in salt concentration from 0 mM to 2 M. In some embodiments, the linear salt gradient comprises an increase in salt concentration from 0 mM to 1 M. In some embodiments, the linear salt gradient comprises an increase in salt concentration from 0 mM to 500 mM. In some embodiments, the linear salt gradient comprises an increase in salt concentration from 0 mM to 250 mM. In some embodiments, the linear salt gradient comprises an increase in salt concentration from 0 mM to 200 mM.

The linear salt gradients described herein may employ any salt suitable for desorbing impurities from a chromatography medium. For example, in some embodiments, the linear salt gradient is an NaCl gradient, a KCl gradient, a CaCl₂ gradient, or an Na₂SO₄ gradient. In some embodiments, the linear salt gradient is an NaCl gradient. In some embodiments, the linear salt gradient is a KCl gradient. In some embodiments, the linear salt gradient is a CaCl₂ gradient. In some embodiments, the linear salt gradient is an Na₂SO₄ gradient.

In some embodiments, the linear salt gradient is a NaCl gradient and comprises an increase in NaCl concentration from 0 mM to 2 M, or 0 mM to 1.9 M, or 0 mM to 1.8 M, or 0 mM to 1.7 M, or 0 mM to 1.6 M, or 0 mM to 1.5 M, or 0 mM to 1.4 M, or 0 mM to 1.3 M, or 0 mM to 1.2 M, or 0 mM to 1.1 M, or 0 mM to 1 M, or 0 mM to 950 mM, or 0 mM to 900 mM, or 0 mM to 850 mM, or 0 mM to 800 mM, or 0 mM to 750 mM, or 0 mM to 700 mM, or 0 mM to 650 mM, or 0 mM to 600 mM, or 0 mM to 550 mM, or 0 mM to 500 mM, or 0 mM to 450 mM, or 0 mM to 400 mM, or 0 mM to 350 mM, or 0 mM to 300 mM, or 0 mM to 250 mM, or 0 mM to 200 mM. In some embodiments, 0 mM in the any of the preceding ranges may be replaced by 5 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM. In some embodiments, the linear salt gradient comprises an increase in NaCl concentration from 0 mM to 2 M. In some embodiments, the linear salt gradient comprises an increase in NaCl concentration from 0 mM to 1 M. In some embodiments, the linear salt gradient comprises an increase in NaCl concentration from 0 mM to 500 mM. In some embodiments, the linear salt gradient comprises an increase in NaCl concentration from 0 mM to 250 mM. In some embodiments, the linear salt gradient comprises an increase in NaCl concentration from 0 mM to 200 mM.

In some embodiments, the linear salt gradient is a KCl gradient and comprises an increase in KCl concentration from 0 mM to 2 M, or 0 mM to 1.9 M, or 0 mM to 1.8 M, or 0 mM to 1.7 M, or 0 mM to 1.6 M, or 0 mM to 1.5 M, or 0 mM to 1.4 M, or 0 mM to 1.3 M, or 0 mM to 1.2 M, or 0 mM to 1.1 M, or 0 mM to 1 M, or 0 mM to 950 mM, or 0 mM to 900 mM, or 0 mM to 850 mM, or 0 mM to 800 mM, or 0 mM to 750 mM, or 0 mM to 700 mM, or 0 mM to 650 mM, or 0 mM to 600 mM, or 0 mM to 550 mM, or 0 mM to 500 mM, or 0 mM to 450 mM, or 0 mM to 400 mM, or 0 mM to 350 mM, or 0 mM to 300 mM, or 0 mM to 250 mM, or 0 mM to 200 mM. In some embodiments, 0 mM in the any of the preceding ranges may be replaced by 5 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM. In some embodiments, the linear salt gradient comprises an increase in KCl concentration from 0 mM to 2 M. In some embodiments, the linear salt gradient comprises an increase in KCl concentration from 0 mM to 1 M. In some embodiments, the linear salt gradient comprises an increase in KCl concentration from 0 mM to 500 mM. In some embodiments, the linear salt gradient comprises an increase in KCl concentration from 0 mM to 250 mM. In some embodiments, the linear salt gradient comprises an increase in KCl concentration from 0 mM to 200 mM.

In some embodiments, the linear salt gradient is a CaCl₂ gradient and comprises an increase in CaCl₂ concentration from 0 mM to 2 M, or 0 mM to 1.9 M, or 0 mM to 1.8 M, or 0 mM to 1.7 M, or 0 mM to 1.6 M, or 0 mM to 1.5 M, or 0 mM to 1.4 M, or 0 mM to 1.3 M, or 0 mM to 1.2 M, or 0 mM to 1.1 M, or 0 mM to 1 M, or 0 mM to 950 mM, or 0 mM to 900 mM, or 0 mM to 850 mM, or 0 mM to 800 mM, or 0 mM to 750 mM, or 0 mM to 700 mM, or 0 mM to 650 mM, or 0 mM to 600 mM, or 0 mM to 550 mM, or 0 mM to 500 mM, or 0 mM to 450 mM, or 0 mM to 400 mM, or 0 mM to 350 mM, or 0 mM to 300 mM, or 0 mM to 250 mM, or 0 mM to 200 mM. In some embodiments, 0 mM in the any of the preceding ranges may be replaced by 5 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM. In some embodiments, the linear salt gradient comprises an increase in CaCl₂ concentration from 0 mM to 2 M. In some embodiments, the linear salt gradient comprises an increase in CaCl₂ concentration from 0 mM to 1 M. In some embodiments, the linear salt gradient comprises an increase in CaCl₂ concentration from 0 mM to 500 mM. In some embodiments, the linear salt gradient comprises an increase in CaCl₂ concentration from 0 mM to 250 mM. In some embodiments, the linear salt gradient comprises an increase in CaCl₂ concentration from 0 mM to 200 mM.

In some embodiments, the linear salt gradient is a Na₂SO₄ gradient and comprises an increase in Na₂SO₄ concentration from 0 mM to 2 M, or 0 mM to 1.9 M, or 0 mM to 1.8 M, or 0 mM to 1.7 M, or 0 mM to 1.6 M, or 0 mM to 1.5 M, or 0 mM to 1.4 M, or 0 mM to 1.3 M, or 0 mM to 1.2 M, or 0 mM to 1.1 M, or 0 mM to 1 M, or 0 mM to 950 mM, or 0 mM to 900 mM, or 0 mM to 850 mM, or 0 mM to 800 mM, or 0 mM to 750 mM, or 0 mM to 700 mM, or 0 mM to 650 mM, or 0 mM to 600 mM, or 0 mM to 550 mM, or 0 mM to 500 mM, or 0 mM to 450 mM, or 0 mM to 400 mM, or 0 mM to 350 mM, or 0 mM to 300 mM, or 0 mM to 250 mM, or 0 mM to 200 mM. In some embodiments, 0 mM in the any of the preceding ranges may be replaced by 5 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM. In some embodiments, the linear salt gradient comprises an increase in Na₂SO₄ concentration from 0 mM to 2 M. In some embodiments, the linear salt gradient comprises an increase in Na₂SO₄ concentration from 0 mM to 1 M. In some embodiments, the linear salt gradient comprises an increase in Na₂SO₄ concentration from 0 mM to 500 mM. In some embodiments, the linear salt gradient comprises an increase in Na₂SO₄ concentration from 0 mM to 250 mM. In some embodiments, the linear salt gradient comprises an increase in Na₂SO₄ concentration from 0 mM to 200 mM.

Depending on the pH of the chromatography operation, suitable buffers for generating the salt gradient include, but are not limited to, acetate buffers, phosphate buffers, sulfate buffers, carbonate buffers, piperazine buffers, imidazole buffers, Tris buffers, MES buffers, and combinations of any of the foregoing. In some embodiments, at least one buffer used to generate the salt gradient has substantially the same composition as the post-load wash solution.

In some embodiments, at least one buffer used to generate the salt gradient comprises acetate. In some embodiments, the at least one buffer comprises at least 5 mM, at least 10 mM, at least 15 mM, at least 20 mM, at least 25 mM, at least 30 mM, at least 35 mM, at least 40 mM, at least 45 mM, at least 50 mM, at least 55 mM, at least 60 mM, at least 65 mM, at least 70 mM, at least 75 mM, at least 80 mM, at least 85 mM, at least 90 mM, at least 95 mM, at least 100 mM, at least 150 mM, at least 200 mM, at least 250 mM, at least 300 mM, at least 350 mM, at least 400 mM, at least 450 mM, at least 500 mM, at least 550 mM, at least 600 mM, at least 650 mM, at least 700 mM, at least 750 mM, at least 800 mM, at least 850 mM, at least 900 mM, at least 950 mM, or at least 1 M acetate.

In some embodiments, each buffer used to generate the salt gradient comprises acetate. In some embodiments, each buffer comprises at least 5 mM, at least 10 mM, at least 15 mM, at least 20 mM, at least 25 mM, at least 30 mM, at least 35 mM, at least 40 mM, at least 45 mM, at least 50 mM, at least 55 mM, at least 60 mM, at least 65 mM, at least 70 mM, at least 75 mM, at least 80 mM, at least 85 mM, at least 90 mM, at least 95 mM, at least 100 mM, at least 150 mM, at least 200 mM, at least 250 mM, at least 300 mM, at least 350 mM, at least 400 mM, at least 450 mM, at least 500 mM, at least 550 mM, at least 600 mM, at least 650 mM, at least 700 mM, at least 750 mM, at least 800 mM, at least 850 mM, at least 900 mM, at least 950 mM, or at least 1 M acetate.

In some embodiments, at least one buffer used to generate the salt gradient comprises 25 mM. 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, 150 mM, 200 mM, 250 mM, 300 mM, 350 mM, 400 mM, 450 mM, 500 mM, 550 mM, 600 mM, 650 mM, 700 mM, 750 mM, 800 mM, 850 mM, 900 mM, 950 mM, or 1M acetate. In some embodiments, each buffer used to generate the salt gradient comprises 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, 150 mM, 200 mM, 250 mM, 300 mM, 350 mM, 400 mM, 450 mM, 500 mM, 550 mM, 600 mM, 650 mM, 700 mM, 750 mM, 800 mM, 850 mM, 900 mM, 950 mM, or 1 M acetate.

In some embodiments, at least one buffer used to generate the salt gradient comprises acetate at a concentration of at least 500 mM. In some embodiments, at least one buffer used to generate the salt gradient comprises acetate at a concentration of at least 100 mM. In some embodiments, at least one buffer used to generate the salt gradient comprises acetate at a concentration in the range of 50 mM to 100 mM. In some embodiments, at least one buffer used to generate the salt gradient comprises acetate at a concentration of at least 50 mM. In some embodiments, at least one buffer used to generate the salt gradient comprises acetate at a concentration of at least 55 mM. In some embodiments, at least one buffer used to generate the salt gradient comprises acetate at a concentration of at least 60 mM. In some embodiments, at least one buffer used to generate the salt gradient comprises acetate at a concentration of at least 65 mM. In some embodiments, at least one buffer used to generate the salt gradient comprises acetate at a concentration of at least 70 mM. In some embodiments, at least one buffer used to generate the salt gradient comprises acetate at a concentration of at least 75 mM. In some embodiments, at least one buffer used to generate the salt gradient comprises acetate at a concentration of at least 80 mM. In some embodiments, at least one buffer used to generate the salt gradient comprises acetate at a concentration of at least 85 mM. In some embodiments, at least one buffer used to generate the salt gradient comprises acetate at a concentration of at least 90 mM. In some embodiments, at least one buffer used to generate the salt gradient comprises acetate at a concentration of at least 95 mM.

In some embodiments, at least one buffer used to generate the salt gradient comprises acetate at a concentration of 100 mM. In some embodiments, at least one buffer used to generate the salt gradient comprises acetate at a concentration of 50 mM, 51 mM, 52 mM, 53 mM, 54 mM, 55 mM, 56 mM, 57 mM, 58 mM, 59 mM, 60 mM, 65 mM, 70 mM, 75 mM, 80 mM, 85 mM, 90 mM, 95 mM, or 100 mM. In some embodiments, at least one buffer used to generate the salt gradient comprises acetate at a concentration of 50 mM. In some embodiments, at least one buffer used to generate the salt gradient comprises acetate at a concentration of 51 mM. In some embodiments, at least one buffer used to generate the salt gradient comprises acetate at a concentration of 52 mM. In some embodiments, at least one buffer used to generate the salt gradient comprises acetate at a concentration of 53 mM. In some embodiments, at least one buffer used to generate the salt gradient comprises acetate at a concentration of 54 mM. In some embodiments, at least one buffer used to generate the salt gradient comprises acetate at a concentration of 55 mM. In some embodiments, at least one buffer used to generate the salt gradient comprises acetate at a concentration of 56 mM. In some embodiments, at least one buffer used to generate the salt gradient comprises acetate at a concentration of 57 mM. In some embodiments, at least one buffer used to generate the salt gradient comprises acetate at a concentration of 58 mM. In some embodiments, at least one buffer used to generate the salt gradient comprises acetate at a concentration of 59 mM. In some embodiments, at least one buffer used to generate the salt gradient comprises acetate at a concentration of 60 mM. In some embodiments, at least one buffer used to generate the salt gradient comprises acetate at a concentration of 65 mM. In some embodiments, at least one buffer used to generate the salt gradient comprises acetate at a concentration of 70 mM. In some embodiments, at least one buffer used to generate the salt gradient comprises acetate at a concentration of 75 mM. In some embodiments, at least one buffer used to generate the salt gradient comprises acetate at a concentration of 80 mM. In some embodiments, at least one buffer used to generate the salt gradient comprises acetate at a concentration of 85 mM. In some embodiments, at least one buffer used to generate the salt gradient comprises acetate at a concentration of 90 mM. In some embodiments, at least one buffer used to generate the salt gradient comprises acetate at a concentration of 95 mM. In some embodiments, at least one buffer used to generate the salt gradient comprises acetate at a concentration of 100 mM.

In some embodiments, each buffer used to generate the salt gradient independently comprises acetate at a concentration of at least 500 mM. In some embodiments, each buffer used to generate the salt gradient independently comprises acetate at a concentration of at least 100 mM. In some embodiments, each buffer used to generate the salt gradient independently comprises acetate at a concentration in the range of 50 mM to 100 mM. In some embodiments, each buffer used to generate the salt gradient independently comprises acetate at a concentration of at least 50 mM. In some embodiments, each buffer used to generate the salt gradient independently comprises acetate at a concentration of at least 55 mM. In some embodiments, each buffer used to generate the salt gradient independently comprises acetate at a concentration of at least 60 mM. In some embodiments, each buffer used to generate the salt gradient independently comprises acetate at a concentration of at least 65 mM. In some embodiments, each buffer used to generate the salt gradient independently comprises acetate at a concentration of at least 70 mM. In some embodiments, each buffer used to generate the salt gradient independently comprises acetate at a concentration of at least 75 mM. In some embodiments, each buffer used to generate the salt gradient independently comprises acetate at a concentration of at least 80 mM. In some embodiments, each buffer used to generate the salt gradient independently comprises acetate at a concentration of at least 85 mM. In some embodiments, each buffer used to generate the salt gradient independently comprises acetate at a concentration of at least 90 mM. In some embodiments, each buffer used to generate the salt gradient independently comprises acetate at a concentration of at least 95 mM.

In some embodiments, each buffer used to generate the salt gradient comprises acetate at a concentration of at least 500 mM. In some embodiments, each buffer used to generate the salt gradient comprises acetate at a concentration of at least 100 mM. In some embodiments, each buffer used to generate the salt gradient comprises acetate at a concentration in the range of 50 mM to 100 mM. In some embodiments, each buffer used to generate the salt gradient comprises acetate at a concentration of at least 50 mM. In some embodiments, each buffer used to generate the salt gradient comprises acetate at a concentration of at least 55 mM. In some embodiments, each buffer used to generate the salt gradient comprises acetate at a concentration of at least 60 mM. In some embodiments, each buffer used to generate the salt gradient comprises acetate at a concentration of at least 65 mM. In some embodiments, each buffer used to generate the salt gradient comprises acetate at a concentration of at least 70 mM. In some embodiments, each buffer used to generate the salt gradient comprises acetate at a concentration of at least 75 mM. In some embodiments, each buffer used to generate the salt gradient comprises acetate at a concentration of at least 80 mM. In some embodiments, each buffer used to generate the salt gradient comprises acetate at a concentration of at least 85 mM. In some embodiments, each buffer used to generate the salt gradient comprises acetate at a concentration of at least 90 mM. In some embodiments, each buffer used to generate the salt gradient comprises acetate at a concentration of at least 95 mM.

In some embodiments, each buffer used to generate the salt gradient comprises acetate at a concentration of 100 mM. In some embodiments, each buffer used to generate the salt gradient comprises acetate at a concentration of 50 mM, 51 mM, 52 mM, 53 mM, 54 mM, 55 mM, 56 mM, 57 mM, 58 mM, 59 mM, 60 mM, 65 mM, 70 mM, 75 mM, 80 mM, 85 mM, 90 mM, 95 mM, or 100 mM. In some embodiments, each buffer used to generate the salt gradient comprises acetate at a concentration of 50 mM. In some embodiments, each buffer used to generate the salt gradient comprises acetate at a concentration of 51 mM. In some embodiments, each buffer used to generate the salt gradient comprises acetate at a concentration of 52 mM. In some embodiments, each buffer used to generate the salt gradient comprises acetate at a concentration of 53 mM. In some embodiments, each buffer used to generate the salt gradient comprises acetate at a concentration of 54 mM. In some embodiments, each buffer used to generate the salt gradient comprises acetate at a concentration of 55 mM. In some embodiments, each buffer used to generate the salt gradient comprises acetate at a concentration of 56 mM. In some embodiments, each buffer used to generate the salt gradient comprises acetate at a concentration of 57 mM. In some embodiments, each buffer used to generate the salt gradient comprises acetate at a concentration of 58 mM. In some embodiments, each buffer used to generate the salt gradient comprises acetate at a concentration of 59 mM. In some embodiments, each buffer used to generate the salt gradient comprises acetate at a concentration of 60 mM. In some embodiments, each buffer used to generate the salt gradient comprises acetate at a concentration of 65 mM. In some embodiments, each buffer used to generate the salt gradient comprises acetate at a concentration of 70 mM. In some embodiments, each buffer used to generate the salt gradient comprises acetate at a concentration of 75 mM. In some embodiments, each buffer used to generate the salt gradient comprises acetate at a concentration of 80 mM. In some embodiments, each buffer used to generate the salt gradient comprises acetate at a concentration of 85 mM. In some embodiments, each buffer used to generate the salt gradient comprises acetate at a concentration of 90 mM. In some embodiments, each buffer used to generate the salt gradient comprises acetate at a concentration of 95 mM. In some embodiments, each buffer used to generate the salt gradient comprises acetate at a concentration of 100 mM.

In some embodiments, each buffer used to generate the salt gradient comprises the same amount of acetate. In some embodiments, each buffer used to generate the salt gradient has the same composition, excluding the amount of salt in the buffer.

In some embodiments, the linear salt gradient cleaning is performed at a pH similar to or compatible with the chromatography operation. In some embodiments, the cleaning is performed at a pH of at least 3.6. In some embodiments, the pH is in the range of 3.6 to 6.0. In some embodiments, the pH is in the range of 4.0 to 5.6. In some embodiments, the pH is in the range of 4.5 to 5.6. In some embodiments, the pH is in the range of 5.0 to 5.6. In some embodiments, the pH is in the range of 5.5 to 5.6. In some embodiments, the pH is at least 4.0. In some embodiments, the pH is at least 4.1. In some embodiments, the pH is at least 4.2. In some embodiments, the pH is at least 4.3. In some embodiments, the pH is at least 4.4. In some embodiments, the pH is at least 4.5. In some embodiments, the pH is at least 4.6. In some embodiments, the pH is at least 4.7. In some embodiments, the pH is at least 4.8. In some embodiments, the pH is at least 4.9. In some embodiments, the pH is at least 5.0. In some embodiments, the pH is at least 5.1. In some embodiments, the pH is at least 5.2. In some embodiments, the pH is at least 5.3. In some embodiments, the pH is at least 5.4. In some embodiments, the pH is 5.5. In some embodiments, the pH is 5.6. In some embodiments, the pH is 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or 6.0.

In some embodiments, each buffer used to generate the salt gradient has the same pH.

In some embodiments, the linear salt gradient cleaning is performed at a conductivity substantially similar to the conductivity of the post-load wash buffer.

In some embodiments, the linear salt gradient is generated by two buffers: Buffer A and Buffer B. In some embodiments, the linear salt gradient is generated using Buffer A and Buffer B in a gradient from 100% Buffer A (0% Buffer B) to 0% Buffer A (100% Buffer B). In some embodiments, the linear salt gradient is generated using Buffer A and Buffer B in a gradient from 90% Buffer A (10% Buffer B) to 10% Buffer A (90% Buffer B). In some embodiments, the linear salt gradient is generated using Buffer A and Buffer B in a gradient from 80% Buffer A (20% Buffer B) to 20% Buffer A (80% Buffer B).

In some embodiments, Buffer A comprises 50 mM acetate and 0 mM sodium chloride at a pH value of 5.0±0.1. In some embodiments, Buffer B comprises 50 mM acetate and 500 mM sodium chloride at a pH value of 5.0±0.1. In some embodiments, Buffer A comprises 50 mM acetate and 0 mM sodium chloride at a pH value of 5.0±0.1 and Buffer B comprises 50 mM acetate and 500 mM sodium chloride at a pH value of 5.0±0.1.

In some embodiments, Buffer A comprises 50 mM acetate and 20 mM sodium chloride at a pH value of 5.0±0.1. In some embodiments, Buffer A comprises 50 mM acetate and 20 mM sodium chloride at a pH value of 5.0±0.1 and Buffer B comprises 50 mM acetate and 500 mM sodium chloride at a pH value of 5.0±0.1.

### Linear Salt Gradient Length and Slope

The slope of a salt gradient is determined by the change in salt concentration (M) divided by the gradient length (in medium or column volumes (“MV”)): M/MV. When performing a salt gradient, there is a tradeoff between the slope of the gradient and the amount of buffer consumed. Steeper slopes are associated with faster impurity desorption and lower buffer requirements but potentially greater pressure spikes during cleaning. By contrast, shallower slopes are associated with slower impurity desorption and higher buffer requirements but reduced risk of pressure spikes.

In some embodiments, the slope of the linear salt gradient is less than or equal to 0.07 M salt/MV buffer. In some embodiments, the slope of the linear salt gradient is less than or equal to 0.065 M salt/MV buffer. In some embodiments, the slope of the linear salt gradient is less than or equal to 0.0625 M salt/MV buffer. In some embodiments, the slope of the linear salt gradient is less than or equal to 0.06 M salt/MV buffer. In some embodiments, the slope of the linear salt gradient is less than or equal to 0.0575 M salt/MV buffer. In some embodiments, the slope of the linear salt gradient is less than or equal to 0.055 M salt/MV buffer. In some embodiments, the slope of the linear salt gradient is less than or equal to 0.0525 M salt/MV buffer. In some embodiments, the slope of the linear salt gradient is less than or equal to 0.05 M salt/MV buffer.

In some embodiments, the gradient length is at least 7.0 MV of buffer (i.e., at least 7.0 medium volumes (e.g., column volumes (“CVs”)) of buffer are passed through the chromatography medium during the linear salt gradient cleaning). In some embodiments, the gradient length is at least 7.1 MV. In some embodiments, the gradient length is at least 8.0 MV.

In some embodiments, the gradient length is in the range of 7.0 MVs to 8.0 MVs. In some embodiments, the gradient length is in the range of 7.1 MVs to 8.0 MVs.

In some embodiments, the gradient length is 7.0 MV, 7.1 MV, 7.2 MV, 7.3 MV, 7.4 MV, 7.5 MV, 7.6 MV, 7.7 MV, 7.8 MV, 7.9 MV, or 8.0 MV. In some embodiments, the gradient length is 7.0 MV. In some embodiments, the gradient length is 7.1 MV. In some embodiments, the gradient length is 7.2 MV. In some embodiments, the gradient length is 7.3 MV. In some embodiments, the gradient length is 7.4 MV. In some embodiments, the gradient length is 7.5 MV. In some embodiments, the gradient length is 7.6 MV. In some embodiments, the gradient length is 7.7 MV. In some embodiments, the gradient length is 7.8 MV. In some embodiments, the gradient length is 7.9 MV. In some embodiments, the gradient length is 8.0 MV.

In some embodiments, the gradient length is greater than 8.0 MV. In some embodiments, the gradient length is 9 MV. In some embodiments, the gradient length is 10.0 MV.

In some embodiments, the slope of the linear salt gradient is less than or equal to 0.07 M salt/MV buffer and the gradient length is at least 7.1 MV (such, as, e.g., at least 8 MVs). In some embodiments, the slope of the linear salt gradient is less than or equal to 0.065 M salt/MV buffer and the gradient length is at least 7.1 MV.

In some embodiments, the slope of the linear salt gradient is less than or equal to 0.065 M salt/MV buffer and the gradient length is 8 MV.

### Denaturing Cleaning

Denaturing cleaning steps, including isocratic denaturing cleaning steps, can reduce fouling, minimize deterioration of a packed column, enable recycling of the chromatography medium, and/or prolong the lifetime of the chromatography medium. Chromatography medium cleaning methods provided herein may further include one or more denaturing cleaning steps (e.g., isocratic denaturing cleaning steps) that, in addition to removing impurities, also reduce or remove contaminants that contribute to the bioburden load.

In some embodiments, one or more denaturing cleaning (e.g., isocratic denaturing cleaning) steps are performed after each cycle of the chromatography medium. In some embodiments, one or more denaturing cleaning (e.g., isocratic denaturing cleaning) steps are performed after two or more cycles of the chromatography medium. In some embodiments, one or more denaturing cleaning (e.g., isocratic denaturing cleaning) steps are performed following a chromatography batch. In some embodiments, one or more denaturing cleaning (e.g., isocratic denaturing cleaning) steps are performed prior to storage. One or more linear salt gradient non-denaturing cleaning steps and one or more one or more denaturing cleaning (e.g., isocratic denaturing cleaning) may be performed together or separately after one or more cycles (i.e., after the same cycle or different cycles), after one or more batches, and/or prior to storage. In some embodiments, at least one denaturing (e.g., isocratic denaturing) cleaning step is performed in combination with (e.g., following) a non-denaturing linear salt gradient cleaning step. In some embodiments, two or more denaturing cleaning (e.g., isocratic denaturing cleaning) steps are performed following a non-denaturing linear salt gradient cleaning step. In some embodiments, at least one denaturing cleaning (e.g., isocratic denaturing cleaning) step is performed following two or more non-denaturing linear salt gradient cleaning steps. In some embodiments, at least one denaturing cleaning (e.g., isocratic denaturing cleaning) step is performed in combination with (e.g., following) a non-denaturing linear salt gradient cleaning step after one or more batches. In some embodiments, at least one denaturing cleaning (e.g., isocratic denaturing cleaning) step is performed in combination with (e.g., following) a non-denaturing linear salt gradient cleaning step prior to storage.

Denaturing cleaning steps, e.g., isocratic denaturing cleaning steps, are commonly performed under harsher conditions than the non-denaturing linear salt gradient cleaning steps described herein. Accordingly, denaturing cleaning steps may cause degradation of the chromatography medium, so care should be taken when selecting operating conditions. In some embodiments, sodium hydroxide is used for the denaturing cleaning step. In some embodiments, sodium hydroxide at a concentration in the range of 0.5 M to 1.5 M is used in a denaturing solution. In some embodiments, 0.5 M sodium hydroxide is used in a denaturing solution. In some embodiments, 1 M sodium hydroxide is used in a denaturing solution. In some embodiments, 1.5 M sodium hydroxide is used in a denaturing solution.

In some embodiments, the contact time (i.e., the number of medium volumes of denaturing solution/flow rate) for the denaturing cleaning step (e.g., the isocratic denaturing cleaning step) is up to 8 hours. In some embodiments, the contact time is up to 7 hours, up to 6 hours, up to 5 hours, up to 4 hours, up to 3 hours, up to 2 hours, or up to 1 hour. In some embodiments, the contact time is 1 hour, 2 hours, 3 hours, 4 hours, 5 hours, 6 hours, 7 hours, or 8 hours.

In some embodiments, up to 5 medium volumes of a denaturing solution are passed through the chromatography medium. In some embodiments, up to 4 medium volumes of a denaturing solution are passed through the chromatography medium. In some embodiments, up to 3 medium volumes of a denaturing solution are passed through the chromatography medium. In some embodiments, up to 1 medium volume of a denaturing solution is passed through the chromatography medium.

In some embodiments, 1 medium volume, 2 medium volumes, 3 medium volumes, 4 medium volumes, or 5 medium volumes of a denaturing solution is/are passed through the chromatography medium. In some embodiments, 1 medium volume of a denaturing solution is passed through the chromatography medium. In some embodiments, 2 medium volumes of a denaturing solution are passed through the chromatography medium. In some embodiments, 3 medium volumes of a denaturing solution are passed through the chromatography medium. In some embodiments, 4 medium volumes of a denaturing solution are passed through the chromatography medium. In some embodiments, 5 medium volumes of a denaturing solution are passed through the chromatography medium.

In some embodiments, the denaturing solution is used in an isocratic cleaning process.

### Chromatography Medium Storage

Following a desired number of cycles or batches, the chromatography medium may be prepared for short- or long-term storage. In some embodiments, the chromatography medium is prepared for storage following completion of one or more batches. In some embodiments, the chromatography medium is prepared for storage after one or more cycles. In some embodiments, the chromatography medium is prepared for storage by subjecting the chromatography medium to one or more cycles using a load solution without protein.

In some embodiments, the chromatography medium is stored in a storage solution (e.g., a storage solution comprising sodium hydroxide). In some embodiments, the storage solution comprises sodium hydroxide at a concentration in the range of 0.1M to 0.2M. In some embodiments, the storage solution comprises sodium hydroxide at a concentration of 0.1M. In some embodiments, the storage solution comprises sodium hydroxide at a concentration of 0.125M. In some embodiments, the storage solution comprises sodium hydroxide at a concentration of 0.150M. In some embodiments, the storage solution comprises sodium hydroxide at a concentration of 0.175M. In some embodiments, the storage solution comprises sodium hydroxide at a concentration of 0.2M.

In some embodiments, at least 2 medium volumes of the storage solution are applied to the chromatography medium. In some embodiments, at least 3 medium volumes of the storage solution are applied to the chromatography medium. In some embodiments, at least 4 medium volumes of the storage solution are applied to the chromatography medium. In some embodiments, up to 5 medium volumes of the storage solution are applied to the chromatography medium.

In some embodiments, 1, 2, 3, 4, or 5 medium volume(s) of the storage solution is/are applied to the chromatography medium. In some embodiments, 1 medium volume of the storage solution is applied to the chromatography medium. In some embodiments, 2 medium volumes of the storage solution are applied to the chromatography medium. In some embodiments, 3 medium volumes of the storage solution are applied to the chromatography medium. In some embodiments, 4 medium volumes of the storage solution are applied to the chromatography medium. In some embodiments, 5 medium volumes of the storage solution are applied to the chromatography medium.

### Product Quality

Product quality monitoring can optionally be performed in real-time, near real-time, and/or offline for each step in a biomanufacturing process. In some embodiments, product quality is assessed by the presence or absence of clipping, degradation, deamidation, and/or aggregation in the purified product. Clipping refers to the partial cleaving of expressed protein, usually by proteolysis. Degradation refers to the disintegration of a larger entity, such as a peptide or protein, into two smaller entities, where one entity may be significantly larger than the other entity or entities. Deamidation refers to any chemical reaction in which an amine functional group in the side chain of an amino acid (e.g., asparagine or glutamine) is removed or converted to another functional group. Aggregation refers to aggregates of proteins, e.g., as high molecular weight (HMW) species. A non-limiting example product quality is the essential absence or a significant reduction of high molecular weight species. In particular, a non-limiting example product quality is high molecular weight content in the range of 10% to 50%.

Product quality monitoring and measurements can be performed using techniques known to those skilled in the art with commercially available equipment and reagents. Such methods include, but are not limited to, enzyme-linked immunosorbent assays (ELISA) for detecting impurities such as leached Protein A and host cell proteins, gel electrophoresis methods, and quantitative polymerase chain reaction methods for nucleic acids, such as quantitative PCR (qPCR). Size exclusion high performance liquid chromatography (SE-HPLC) and capillary electrophoresis methods, including reduced capillary electrophoresis (rCE-SDS), can be used to measure high molecular weight species (e.g., aggregates) and low molecular weight species, including product monomer, fragments, and unassembled components. Charge variants may be assessed using techniques such as cation exchange high performance liquid chromatography (CEX-HPLC).

### Additional Chromatography Operations

In some embodiments of the present disclosure, one or more chromatography operations can be performed upstream and/or downstream of the frontal chromatography operations described above. In some embodiments, at least one affinity chromatography operation is followed by a single polishing chromatography operation performed in a mode that exhibits a non-linear relationship between step yield and loading density. In some embodiments, at least one affinity chromatography operation is followed by a single polishing chromatography operation performed in frontal mode. In some embodiments, one or more additional polishing chromatography unit operations may be performed before or after the frontal chromatography unit operation. The one or more additional polishing chromatography unit operations may be performed in bind and elute mode, flow through mode, weak partitioning chromatography mode, overload mode, and/or frontal mode. In some embodiments, the frontal chromatography operation is followed by one or more polishing chromatography operations performed in flow through or weak partitioning chromatography modes. In some embodiments, a cation exchange chromatography unit operation is performed in a mode that exhibits a non-linear relationship between step yield and loading density. In some embodiments, a cation exchange chromatography unit operation is performed in frontal mode.

Non-limiting examples of suitable chromatography media for additional polishing chromatography operations include ion exchange chromatography (IEX) media, including cation exchange chromatography (CEX) and anion exchange chromatography (AEX) media, multimodal or mixed-mode chromatography (MMC) media, hydrophobic interaction chromatography (HIC) media, and hydroxyapatite (HA) media.

In addition to chromatography operations, other unit operations can be included in methods described herein, including, but not limited to, filtration operations, such as depth filtration or viral filtration, and viral inactivation operations, including low pH and detergent viral inactivation.

### Host Cells

Cell lines (also referred to as “cells” or “host cells”) used in the present disclosure are genetically engineered to express a protein of commercial or scientific interest. Cells may be suitable for adherent, monolayer, and/or suspension culture, transfection, and expression of recombinant proteins, such as, e.g., antibodies. The cells can be used, for example, with batch, fed batch, and perfusion, or continuous culture methods. Such cells are typically cell lines obtained or derived from mammals and are able to grow and survive when placed in either monolayer culture or suspension culture in medium containing appropriate nutrients and/or other factors, such as those described herein. Host cells are typically selected that can express and secrete proteins, or that can be molecularly engineered to express and secrete, large quantities of a particular protein, more particularly, a glycoprotein of interest, into the culture medium. The selection of an appropriate host cell for expressing a recombinant protein will depend upon various factors, such as desired expression levels, polypeptide modifications that are desirable or necessary for activity (such as glycosylation or phosphorylation), and ease of folding into a biologically active molecule. In some embodiments of the methods of the present disclosure, the host cell is a mammalian host cell.

Cell lines are typically derived from a lineage arising from a primary culture that can be maintained in culture for an unlimited time. The cells can contain introduced, e.g., via transformation, transfection, infection, or injection, expression vectors (constructs), such as plasmids and the like, that harbor coding sequences, or portions thereof, encoding the proteins for expression and production in the culturing process. Such expression vectors contain the necessary elements for the transcription and translation of the inserted coding sequence. Methods which are well known to and practiced by those skilled in the art can be used to construct expression vectors containing sequences encoding the desired proteins and polypeptides, as well as the appropriate transcriptional and translational control elements. These methods include, but are not limited to, in vitro recombinant DNA techniques, synthetic techniques, and in vivo genetic recombination. Such techniques are described in J. Sambrook et al., 2012, Molecular Cloning, A Laboratory Manual, 4^{th} edition Cold Spring Harbor Press, Plainview, N. Y. or any of the previous editions; F. M. Ausubel et al., 2013, Current Protocols in Molecular Biology, John Wiley & Sons, New York, N.Y, or any of the previous editions; Kaufman, R. J., Large Scale Mammalian Cell Culture, 1990, all of which are incorporated herein for any purpose.

Suitable host cells include, but are not limited to, those that are commercially available, for example, from culture collections such as the DSMZ (Deutsche Sammlung von Mikroorganismen and Zellkulturen GmbH, Braunschweig, Germany) or the American Type Culture Collection (ATCC).

Example host cells include, but are not limited to, prokaryote, yeast, or higher eukaryote cells. Prokaryotic host cells include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacillus, such as B. subtilis and B. licheniformis, Pseudomonas, and Streptomyces. In some embodiments, eukaryotic microbes such as filamentous fungi or yeast are suitable cloning or expression hosts for recombinant polypeptides. Saccharomyces cerevisiae, or common baker's yeast, is the most commonly used among lower eukaryotic host microorganisms. However, a number of other genera, species, and strains are commonly available and useful herein, such as Pichia, e.g. P. pastoris, Schizosaccharomyces pombe; Kluyveromyces, Yarrowia; Candida; Trichoderma reesia; Neurospora crassa; Schwanniomyces, such as Schwanniomyces occidentalis; and filamentous fungi, such as, e.g., Neurospora, Penicillium, Tolypocladium, and Aspergillus hosts such as A. nidulans and A. niger.

Vertebrate host cells are also suitable hosts for expressing recombinant proteins. Mammalian cell lines suitable as hosts for recombinant protein expression are well-known in the art and include, but are not limited to, immortalized cell lines available from the American Type Culture Collection (ATCC), including, but not limited to, Chinese hamster ovary (CHO) cells, including CHOK1 cells (ATCC CCL61), DXB-11, DG-44, and Chinese hamster ovary cells/-DHFR (CHO, Urlaub et al., Proc. Natl. Acad. Sci. USA 77:4216, 1980); monkey kidney CV1 line transformed by SV40 (COS-7, ATCC CRL 1651); human embryonic kidney line (293 or 293 cells subcloned for growth in suspension culture, (Graham et al., J. Gen Virol. 36:59, 1977); baby hamster kidney cells (BHK, ATCC CCL 10); mouse sertoli cells (TM4, Mather, Biol. Reprod. 23:243-251, 1980); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human hepatoma cells (Hep G2, HB 8065); mouse mammary tumor (MMT 060562, ATCC CCL51); TRI cells (Mather et al., Annals N.Y Acad. Sci. 383:44-68, 1982); MRC 5 cells or FS4 cells; mammalian myeloma cells, and a number of other cell lines. In some embodiments, the host cells are selected from CHO cells.

In some embodiments, the host cells are eukaryotic cells, such as, e.g., mammalian cells. The mammalian cells can be, for example, human or rodent or bovine cell lines or cell strains. Examples of such cells, cell lines, or cell strains include, but are not limited to, mouse myeloma (NSO)-cell lines, Chinese hamster ovary (CHO)-cell lines, FIT 1080, H9, HepG2, MCF7, MDBK Jurkat, NIH3T3, PC12, BF1K (baby hamster kidney cell), VERO, SP2/0, YB2/0, Y0, C127, L cell, COS, e.g., COS1 and COS7, QC1-3, HEK-293, VERO, PER.C6, HeLa, EB1, EB2, EB3, oncolytic, or hybridoma-cell lines. In some embodiments, the mammalian cells are CHO-cell lines. In some embodiments, the mammalian cells are CHO cells. In some embodiments, the mammalian cells are selected from CHO-K1 cells, CHO-K1 SV cells, DG44 CHO cells, DUXB11 CHO cells, CHOS cells, CHO GS knock-out cells, CHO FUT8 GS knock-out cells, CHOZN cells, and CHO derived cells. In some embodiments, a CHO GS knock-out cell (such as, e.g., a GSKO cell) is, for example, a CHO-K1 SV GS knockout cell. Additionally, the CHO FUT8 knockout cell is, for example, the Potelligent® CHOK1 SV (Lonza, Inc.). In some embodiments, the eukaryotic cells can also be avian cells, cell lines, or cell strains, such as, e.g., EBx® cells, EB14, EB24, EB26, EB66, or EBv13.

CHO cells, including CHOK1 cells (ATCC CCL61), are widely used to produce complex recombinant proteins. In some embodiments, the dihydrofolate reductase (DHFR)-deficient mutant cell lines (Urlaub et al., 1980, Proc Natl Acad Sci USA 77:4216-4220), DXB11 and DG-44, are desirable CHO host cell lines because the efficient DHFR selectable and amplifiable gene expression system allows high level recombinant protein expression in these cell lines (Kaufman R. J., 1990, Meth Enzymol 185:537-566). Also included are the glutamine synthase (GS)-knockout CHOK1SV cell lines, making use of glutamine synthetase (GS)-based methionine sulfoximine (MSX) selection. Other suitable CHO host cells for use in a bioreactor of a manufacturing process of the present disclosure include, but are not limited to, the following (ECACC accession numbers in parenthesis): CHO (85050302); CHO (PROTEIN FREE) (00102307); CHO-K1 (85051005); CHO-K1/SF (93061607); CHO/dhFr-(94060607); CHO/dhFr-AC-free (05011002); and RR-CHOKI (92052129).

Large-scale production of proteins for commercial applications may be carried out in suspension culture. Therefore, mammalian host cells used to generate proteins of interest can, but need not, be adapted to growth in suspension culture. A variety of host cells adapted to growth in suspension culture are known, including mouse myeloma NSO cells and CLIO cells from CFIO-S, DG44, and DXB11 cell lines. Other suitable cell lines include, but are not limited to, mouse myeloma SP2/0 cells, baby hamster kidney BF1K-21 cells, human PER.C6® cells, human embryonic kidney F1EK-293 cells, and cell lines derived or engineered from any of the cell lines disclosed herein.

In some embodiments, the eukaryotic cells are selected from lower eukaryotic cells, such as, e.g., yeast cells (e.g., Pichia genus (e.g., Pichia pastoris, Pichia methanolica, Pichia kluyveri, and Pichia angusta), Komagataella genus (e.g., Komagataella pastoris, Komagataella pseudopastoris, or Komagataella phaffii), cells of the Saccharomyces genus (e.g., Saccharomyces cerevisae, Saccharomyces kluyveri, Saccharomyces uvarum), cells of the Kluyveromyces genus (e.g., Kluyveromyces lactis, Kluyveromyces marxianus), cells of the Candida genus (e.g., Candida utilis, Candida cacaoi, Candida boidinii), cells of the Geotrichum genus (e.g., Geotrichum fermentans), Hαη-senula polymorpha, Yarrowia lipolytica, or Schizosaccharomyces pombe. In some embodiments, the eukaryotic cells are selected from Pichia pastoris strains. Non-limiting examples of Pichia pastoris strains include X33, GS115, KM71, KM71H, and CBS7435.

In some embodiments, the eukaryotic cells are selected from fungal cells (e.g., cells of Aspergillus (such as, e.g., A. niger, A. fumigatus, A. orzyac, A. nidula), Acremonium (such as, e.g., A. thermophilum), Chaetomium (such as, e.g., C. thermophilum), Chrysosporium (such as, e.g., C. thermophile), Cordyceps (such as, e.g., C. militaris), Corynascus, Ctenomyces, Fusarium (such as, e.g., F. oxysporum), Glomerella (such as, e.g., G. graminicola), Hypocrea (such as, e.g., H. jecorina), Magnaporthe (such as, e.g., M. orzyac), Myceliophthora (such as, e.g., M. thermophile), Nectria (such as, e.g., N. heamatococca), Neurospora (such as, e.g., N. crassa), Penicillium, Sporotrichum (such as, e.g., S. thermophile), Thielavia (such as, e.g., T. terrestris, T. heterothallica), Trichoderma (such as, e.g., T. reesei), or Verticillium (such as, e.g., V. dahlia)).

In some embodiments, the eukaryotic cells are selected from insect cells (such as, e.g., Sf9, Mimic™ Sf9, Sf21, High Five™ (BT1-TN-5B1-4), or BT1-Ea88 cells), algae cells (such as, e.g., of the genus Amphora, Bacillariophyceae, Dunaliella, Chlorella, Chlamydomonas, Cyanophyta (cyanobacteria), Nannochloropsis, Spirulina, or Ochromonas), and plant cells (such as, e.g., cells from monocotyledonous plants (such as, e.g., maize, rice, wheat, or Setaria), or cells from a dicotyledonous plants (such as, e.g., cassava, potato, soybean, tomato, tobacco, alfalfa, Physcomitrella patens or Arabidopsis)).

To generate host cell lines (e.g., mammalian cell lines) engineered to express a recombinant protein of interest, one or more nucleic acids encoding the recombinant protein (or components thereof in the case of multi-chain proteins) is initially inserted into one or more expression vectors. Nucleic acid control sequences useful in expression vectors for expression in mammalian cells include promoters, enhancers, and termination and polyadenylation signals. A secretory signal peptide sequence can also, optionally, be encoded by the expression vector, operably linked to the coding sequence of interest, so that the expressed protein can be secreted by the recombinant host cell, for more facile isolation of the recombinant protein from the cell, if desired. Vectors may also include one or more selectable marker genes to facilitate selection of host cells into which the vectors have been introduced. In some embodiments, vectors are used that employ protein-fragment complementation assays using protein reporters, such as dihydrofolate reductase (see, for example, U.S. Pat. No. 6,270,964). Suitable mammalian expression vectors are known in the art and are also commercially available.

Typically, vectors used in any of the host cells will contain sequences for plasmid maintenance and for cloning and expression of exogenous nucleotide sequences. Such sequences will typically include one or more of the following nucleotide sequences: a promoter, one or more enhancer sequences, an origin of replication, transcriptional and translational control sequences, a transcriptional termination sequence, a complete intron sequence containing a donor and acceptor splice site, a native or heterologous signal peptide sequence (leader sequence or signal peptide) for polypeptide secretion, a ribosome binding site, a polyadenylation sequence, a polylinker region for inserting the polynucleotide encoding the polypeptide to be expressed, and a selectable marker element. Vectors may be constructed from a starting vector such as a commercially available vector, and additional elements may be individually obtained and ligated into the vector.

### Culture Methods

Various culture methods may be used to produce a protein of interest, including, but not limited to, batch culture, fed-batch culture, and perfusion culture.

Batch culture is a discontinuous method where cells are grown in a fixed volume of culture media for a short period of time followed by a full harvest. Cultures grown using the batch method experience an increase in cell density until a maximum cell density is reached, followed by a decline in viable cell density as the media components are consumed and levels of metabolic by-products (such as lactate and ammonia) accumulate. Harvest typically occurs at the point when the maximum cell density is achieved (e.g., 5×10⁶ cells/mL or greater, depending on media formulation, cell line, etc.). The batch process is the simplest culture method; however, viable cell density is limited by nutrient availability and once the cells are at maximum density, the culture declines and production decreases. There is no ability to extend a production phase in batch culture because the accumulation of waste products and nutrient depletion rapidly lead to culture decline, typically around 3 to 7 days.

Fed-batch culture improves on the batch process by providing bolus or continuous media feeds to replenish those media components that have been consumed. Since fed-batch cultures receive additional nutrients throughout the run, they have the potential to achieve higher cell densities (>10 to 30×10⁶ cells/mL, depending on media formulation, cell line, etc.) and increased product titers, when compared to the batch method. Unlike the batch process, a biphasic culture can be created and sustained by manipulating feeding strategies and media formulations to distinguish the period of cell proliferation to achieve a desired cell density (the growth phase) from the period of suspended or slow cell growth (the production phase). As such, fed-batch cultures have the potential to achieve higher product titers compared to batch cultures. Typically, a batch method is used during the growth phase and a fed-batch method used during the production phase, but a fed-batch feeding strategy can be used throughout the entire process. However, unlike the batch process, bioreactor volume is a limiting factor which limits the amount of feed. Also, as with the batch method, metabolic by-product accumulation will lead to culture decline, which limits the duration of the production phase, often around 10 to 21 days. Fed-batch cultures are discontinuous, and harvest typically occurs when metabolic by-product levels or culture viability reach predetermined levels. When compared to a batch culture, in which no feeding occurs, a fed-batch culture can produce greater amounts of recombinant protein. (See, e.g., U.S. Pat. No. 5,672,502.)

Perfusion methods offer potential improvements over the batch and fed-batch methods by adding fresh media and simultaneously removing spent media during culture. Typical perfusion cultures begin with a batch culture start-up lasting for a day or two followed by continuous, step-wise, and/or intermittent addition of fresh feed media to the culture and simultaneous removal of spent media with the retention of cells and additional high molecular weight compounds such as proteins (based on the filter molecular weight cutoff) throughout the growth and production phases of the culture. Various methods, such as sedimentation, centrifugation, or filtration, can be used to remove spent media, while maintaining cell density. Non-limiting example filtration methods include tangential flow filtration (TFF), such as recirculating flow filtration and alternating tangential flow (ATF) filtration. Alternating tangential flow is maintained by pumping medium through hollow-fiber filter modules. See e.g. U.S. Pat. No. 6,544,424; Furey, 2002, Gen. Eng. News. 22 (7): 62-63.

Perfusion can be continuous, stepwise, intermittent, or a combination of any or all of any of these. Perfusion rates can be less than a working volume to many working volumes per day. The cells are retained in the culture, and the spent medium that is removed is substantially free of cells or has significantly fewer cells than the culture. Recombinant proteins expressed by the cell culture can also be retained in the culture.

Another type of perfusion culture, lean perfusion, operates at a high cell density during the growth phase(s) and a low cell density during the production phase(s). A low medium exchange rate sufficient to ensure removal of waste byproducts to avoid culture toxicity while delivering sufficient nutrients via the fresh media to maintain essential cellular functions is maintained throughout the culture, as described in U.S. Provisional Application No. 63/403,896.

Typical large scale commercial cell culture strategies strive to reach high cell densities, 40-90 (+)×10⁶ cells/mL, such as, e.g., about 40×10⁶ cells/mL or about 50×10⁶ cells/mL, where almost a third to over one-half of the reactor volume is biomass. With perfusion culture, extreme cell densities of >1×10⁸ cells/mL have been achieved. A potential advantage of perfusion processes is that the production culture can be maintained for longer periods than batch or fed-batch culture methods. However, increased media preparation, use, storage, and disposal are necessary to support a long-term perfusion culture, particularly for a culture with high cell density, which also needs even more nutrients. In addition, higher cell densities can cause problems during production, such as, e.g., maintaining dissolved oxygen levels and problems with increased gassing, including supplying more oxygen and removing more carbon dioxide, which could result in more foaming and the need for alterations to antifoam strategies; as well as during harvest and downstream processing where the efforts required to remove the excessive cell material can result in loss of product, negating the benefit of increased titer due to increased cell mass.

Suitable culture conditions, including temperature, dissolved oxygen content, agitation rate, and the like, for mammalian cells are known in the art and may vary based on the phase or stage of the cell culture. In some embodiments, the methods disclosed herein further comprise taking samples during the cell culture processes and evaluating the samples to quantitatively and/or qualitatively monitor characteristics of the recombinant protein and/or the cell culture process. In some embodiments, the samples are quantitatively and/or qualitatively monitored using process analytical techniques. For examples, dissolved oxygen levels may be monitored during the cell culture processes using methods known in the art, such as, e.g., a basic chemical analysis method (titration method), an electrochemical analysis method (diaphragm electrode method), and a photochemical analysis method (fluorescence method).

During protein production, it is desirable to have a controlled system where cells are grown for a desired time or to a desired density and then the physiological state of the cells is switched to a growth-limited or arrested, high productivity state where the cells use energy and substrates to produce the recombinant protein in favor of increasing cell density. For commercial scale cell culture and the manufacture of biological therapeutics, the ability to limit or arrest cell growth and to maintain the cells in a growth-limited or arrested state during the production phase is very desirable. Such methods include, for example, temperature shifts, use of chemical inducers of protein production, nutrient limitation or starvation, and cell cycle inhibitors, either alone or in combination. Illustratively, a typical cell culture undergoes a growth phase, a period of exponential growth where cell density is increased. During the growth phase, cells are cultured in a cell culture medium containing the necessary nutrients and additives under conditions (generally at about a temperature of 25°-40° C., in a humidified, controlled atmosphere) such that optimal growth is achieved for the particular cell line. Cells are typically maintained in the growth phase for a period of between one and eight days, e.g., between three to seven days, e.g., seven days. The length of the growth phase for a particular cell line can be determined by a person of ordinary skill in the art and will generally be the period of time sufficient to allow the particular cells to reproduce to a viable cell density within a range of about 20%-80% of the maximal possible viable cell density if the culture was maintained under the growth conditions. The growth phase is followed by a transition phase when exponential cell growth is slowing and protein production starts to increase. This marks the start of the stationary phase, a production phase, where cell density typically levels off and product titer increases. During the production phase, the medium is generally supplemented to support continued recombinant protein production.

In certain embodiments of the methods of the present disclosure, the culture conditions may be adjusted to facilitate the transition from the growth phase of the cell culture to the production phase. For instance, a growth phase of the cell culture may occur at a higher temperature than a production phase of the cell culture. In some embodiments, a growth phase may occur at a first temperature from about 35° C. to about 38° C., and a production phase may occur at a second temperature from about 29° C. to about 37° C., optionally from about 30° C. to about 36° C. or from about 30° C. to about 34° C. In some embodiments, a shift in temperature from about 35° C. to about 37° C. to a temperature of about 31° C. to about 33° C. may be employed to facilitate the transition from the growth phase of the culture to the production phase. Chemical inducers of protein production, such as, for example, caffeine, butyrate, and hexamethylene bisacetamide (HMBA), may be added at the same time as, before, and/or after a temperature shift, or in place of a temperature shift. If inducers are added after a temperature shift, they can be added from one hour to five days after the temperature shift, optionally from one to two days after the temperature shift.

Additionally, any cell culture media capable of supporting growth of the appropriate host cell in culture can be used. Typically, the cell culture medium contains a buffer, salts, energy source, amino acids, vitamins, and trace essential elements. Cell culture media, which may be further supplemented with other components to maximize cell growth, cell viability, and/or recombinant protein production in a particular cultured host cell, are commercially available and include RPMI-1640 Medium, RPMI-1641 Medium, Dulbecco's Modified Eagle's Medium (DMEM), Minimum Essential Medium Eagle, F-12K Medium, Ham's F12 Medium, Iscove's Modified Dulbecco's Medium, McCoy's 5A Medium, Leibovitz's L-15 Medium, and serum-free media such as EX-CELL™ 300 Series, among others, which can be obtained from the American Type Culture Collection or SAFC Biosciences, as well as other vendors. Cell culture media can be serum-free, protein-free, growth factor-free, and/or peptone-free media. Cell culture media may also be enriched by the addition of nutrients or other supplements, which may be used at greater than usual, recommended concentrations. In certain embodiments, the culture medium used in the methods of the present disclosure is a chemically defined medium, which refers to a cell culture medium in which all of the components have known chemical structures and concentrations. Chemically defined media are typically serum-free and do not contain hydrolysates or animal-derived components.

Various media formulations can be used during the life of the culture, for example, to facilitate the transition from one stage (e.g., the growth stage or phase) to another (e.g., the production stage or phase) and/or to optimize conditions during cell culture (e.g. concentrated media provided during a perfusion culture). A growth medium formulation can be used to promote cell growth and minimize protein expression. A production medium formulation can be used to promote production of the recombinant protein of interest and maintenance of the cells, with minimal new cell growth. A feed medium is typically a cell culture medium containing more concentrated components such as nutrients and amino acids, which are consumed during the course of the production phase of the cell culture. A feed medium may be used to supplement and maintain an active culture, particularly a culture operated in fed batch, semi-perfusion, or perfusion mode. Such a concentrated feed medium can contain most of the components of the cell culture medium at, for example, about 5×, 6×, 7×, 8×, 9×, 10×, 12×, 14×, 16×, 20×, 30×, 50×, 100×, 200×, 400×, 600×, 800×, or even about 1000× of their normal amount.

In some embodiments of the methods of the present disclosure, a mammalian cell is cultured for a defined period of time during which the recombinant protein is expressed and secreted by the mammalian cell. This period of time (i.e., the duration of the production phase of the cell culture) is at least 3 days, at least 7 days, at least 10 days, or at least 15 days. In certain embodiments, the duration of the production phase of the cell culture is about 7 days to 28 days, 10 days to 30 days, 7 days to 14 days, 10 days to 18 days, 3 days to 15 days, 5 days to 8 days, 12 days to 15 days, 12 days to 18 days, or 15 days to 21 days. In some embodiments, the duration of the production phase of the cell culture is 7 days, 8 days, 9 days, 12 days, 15 days, 18 days, or 21 days.

In some embodiments, certain methods of the present disclosure comprise a production phase with a viable cell density of at least 100×10⁵ cells/mL, for example, between about 100×10⁵ cells/mL and about 10×10⁷ cells/mL, between about 250×10⁵ cells/mL and about 900×10⁵ cells/mL, between about 300×10⁵ cells/mL and 800×10⁵ cells/mL, or between about 450×10⁵ cells/mL and 650×10⁵ cells/mL. Cell density may be measured using a hemacytometer, a Coulter counter, or an automated cell analyzer (e.g. Cedex automated cell counter). Viable cell density may be determined by staining a culture sample with Trypan blue, which is taken up only by dead cells. Viable cell density is then determined by counting the total number of cells, dividing the number of stained cells by the total number of cells, and taking the reciprocal.

### Bioreactors

Cells may be cultured in suspension or in an adherent form, attached to a solid substrate. Cells can be established with or without microcarriers.

In some embodiments, cells may be cultured in a bioreactor. The bioreactor may comprise a disposable container, e.g., made of a plastic material, or a reusable container, e.g., made of glass or stainless steel. In some embodiments, cell culture methods of the present disclosure are conducted in stainless-steel bioreactors, such as, e.g., built-in-place large-scale stainless-steel bioreactors.

In some embodiments, the bioreactor can have a volume in the range of 100 mL to 50,000 L. Unless otherwise indicated, a bioreactor can be of any size so long as it is useful for the culturing of cells; typically, a bioreactor is sized appropriate to the volume of cell culture being grown inside of it. In non-limiting embodiments and unless otherwise indicated by context, a bioreactor may be at least 500 L, 1,000 L, 1,500 L, 2,000 L, 2,500 L, 5,000 L, 8,000 L, 10,000 L, 12,000 L, 18,000 L, 20,000 L more, or any volume in between.

The internal conditions of the bioreactor, including, but not limited to, pH and temperature, can be controlled during the culturing period. Those of ordinary skill in the art will be aware of, and will be able to select, suitable bioreactors for use in cell culture methods disclosed herein based on the relevant considerations.

In some embodiments, a bioreactor can perform one or more (e.g., one, two, three, all) of the following steps: feeding of nutrients and/or carbon sources; injection of suitable gas (such as, e.g., oxygen); inlet and outlet flow of cell culture medium; separation of gas and liquid phases; maintenance of temperature; maintenance of oxygen and CO₂ levels; maintenance of pH level, agitation (e.g., stirring); and/or cleaning/sterilizing.

### Harvest

During and/or following a production phase of a cell culture, the culture is harvested. For example, the contents of a bioreactor may be partially harvested one or more times during a production phase. Alternatively, the contents of a bioreactor may be fully harvested at the conclusion of a production phase. The harvesting operation fully or partially clarifies and/or separates the protein of interest away from at least one impurity with which it is found in the cell culture fluid, such as, e.g., remaining cell culture media, cells, cell debris, undesired cell, or media components, and/or product- and/or process-related impurities. Methods for harvesting recombinant proteins from suspension cell cultures are known in the art and include, but are not limited to, precipitation, such as acid precipitation, accelerated sedimentation such as flocculation, separation using gravity, centrifugation, acoustic wave separation, filtration including membrane filtration, ultrafiltration, microfiltration, tangential flow filtration, alternative tangential flow filtration, depth filtration, and alluvial filtration (for example, see U.S. Pat. Nos. 9,371,554, 11,384,378).

The harvested cell culture fluid (HCCF) can be stored in surge tanks, holding tanks, bags, or other containers that are adapted to provide feed to a downstream filtration or chromatography operation and are appropriate for the infrastructure and/or process requirements. HCCF may also be fed directly and continuously to a downstream operation.

### Affinity Chromatography

Affinity chromatography is commonly used to perform an initial separation of a protein of interest away from contaminants and impurities, such as product- and/or process-related impurities, in a crude or clarified load stream or pool. Affinity chromatography makes use of agents that bind and/or interact in some manner with at least one desired protein, impurity, and/or contaminant.

In some embodiments, harvested cell culture fluid is subjected to an affinity chromatography operation to isolate and concentrate a desired protein, e.g., a desired protein having a Fc (fragment crystallizing) component, from the harvested cell culture fluid.

Non-limiting examples of affinity chromatography media include Staphylococcus aureus proteins such as Protein A, Protein G, Protein A/G, and Protein L; substrate-binding capture mechanisms; antibody- or antibody fragment-binding capture mechanisms; aptamer-binding capture mechanisms; cofactor-binding capture mechanisms; and the like. Immobilized metal affinity chromatography (IMAC) can be used to capture proteins that have or have been engineered to have affinity for metal ions. Protein A affinity chromatography is typically used in first-line, bulk purification operations for Fc (Fragment, crystallizable) region-containing proteins. Protein A ligands are highly selective for a wide range of proteins and provide high target protein yields and robust removal of process-related impurities. Suitable Protein A methods and materials are widely known and available.

In some embodiments, the Protein A chromatography medium is a high capacity chromatography medium, such as MABSELECT™ PrismA. In some embodiments, the Protein A chromatography medium is loaded up to a loading density of 65 g/L-r.

An affinity chromatography operation may consist of a single independent skid that is operated one or more times to obtain a desired volume of product pool. An affinity chromatography operation may also include two or more independent skids that are operated simultaneously in parallel. An non-limiting example of an automated, parallel chromatography system is described in WO2022/191971 and WO2022/081939. In some embodiments, two or more affinity chromatography columns may be operated in series in a single skid. There are a variety of multi-column cycling strategies, such as closed loop simulated moving bed multi-column systems, sequential multi-column chromatography, an example of which is periodic counter-current chromatography (PCC), and the like. Affinity chromatography operations with multiple skids may make use of the same or similar chromatography media.

Affinity chromatography, such as Protein A affinity chromatography, is typically preformed to clarify harvested cell culture fluid at a neutral pH. The affinity chromatography medium may be equilibrated with a suitable buffer prior to being contacted with load material containing the recombinant protein to be purified. A stream or pool containing the product of interest is directly loaded onto the affinity chromatography medium under conditions that promote binding of the protein.

Once the product of interest is loaded and bound, the chromatography medium is optionally washed with one or more wash buffers prior to the elution step. The wash step(s) can be performed to bind any product of interest that is still on the column but not yet bound to the chromatography medium, flush out load material from the interstitial spaces, remove impurities that have bound to and/or are within the chromatography medium, impurities that have bound to the product of interest. The wash can also be used to prepare the column for elution. Multiple wash buffers may be used depending on the purpose and number of the wash steps. Where multiple wash steps are used, the composition and/or concentration of wash buffer formulations may be the same or different as needed. Washes are performed at appropriate pH, typically at neutral pH, but can be performed at higher or lower pH as needed or desired.

The bound product of interest may be eluted from the chromatography medium by altering the buffer conditions. Various elution buffer formulations are known and used, depending on the characteristics of the product of interest. Elution from affinity chromatography medium is typically performed via an isocratic gradient performed under low pH conditions. In some embodiments, elution from the affinity chromatography is by a linear pH gradient. In some embodiments, the affinity chromatography medium is a Protein A affinity chromatography medium.

Cell culture harvest material may be directly and continuously loaded onto one or more affinity chromatography skids via an effluent stream from a harvest operation or bulk loaded from harvest hold tanks or pools.

### Viral Inactivation

Unit operations directed towards inactivating, reducing, and/or eliminating viral contaminants may include processes that mitigate viral risk by manipulating the environment and/or through the use of filtration. Viral mitigation measures are important to ensure the safety of protein therapeutics and may be performed one or more times throughout a downstream purification process. Viral contaminants can arise from a variety of sources including the use of reagents of animal origin, adventitious viral contaminants in host cell lines, or system failures at GMP manufacturing sites. Viruses are classified as enveloped and non-enveloped viruses. With enveloped viruses, the envelope allows the virus to identify, bind, enter, and infect target host cells. As such, enveloped viruses are susceptible to inactivation methods. Various methods can be employed for viral inactivation including heat inactivation/pasteurization, UV and gamma ray irradiation, use of high intensity broad spectrum white light, addition of chemical inactivating agents, surfactants, and solvent/detergent treatments. Surfactants, such as detergents, solubilize membranes and therefore can be very effective in specifically inactivating enveloped viruses, see, e.g., WO2020/190985.

Another method for achieving viral inactivation is incubation at low pH (e.g., pH<4). Low pH viral inactivation can be followed by neutralization that readjusts the acidic viral inactivated solution to a pH more compatible for the following downstream operations. Low pH viral inactivation is typically performed following purification of the harvest cell culture fluid with affinity chromatography, in particular affinity chromatography that makes use of a substrate binding ligand from Staphylococcus aureus, such as Protein A chromatography, since elution is typically performed at a low pH. Bulk viral inactivation takes place in one or more hold tanks; an non-limiting example process for an automated two tank low pH inactivation and neutralization operation is described in WO2022/099162. Low pH viral inactivation may also be followed by filtration, such as depth filtration, for clarification of the neutralized fluid and/or sterile filtration.

Another method is to elute a Protein A affinity chromatography medium at pH sufficient for viral inactivation. The acidified elution pool or stream serves as the load feed for a polishing chromatography operation in frontal loading mode, with the loading time, rather than the flow between unit operations, being adjusted to meet at least the minimal residence time required for effective viral inactivation of the contents of the frontal chromatography eluted product pool. This process eliminates the need for hold tanks and allows for a continuous, connected flow between chromatography unit operations. Additionally, this process minimizes buffer volumes and eliminates further post-elution processing, creating a more streamlined, efficient, and robust process. These conditions are not only effective for viral inactivation but may increase product yield and decrease product-related impurities in the product pool from the frontal chromatography operation. The acidified elution pool or stream can be passed through one or more depth and/or sterilization filters prior to frontal loading.

This method may also be advantageous over either a bulk pool or continuous in-line viral inactivation strategy in that it supports a continuous process by maintaining a constant flow from one unit operation to another. Constant flow minimizes the time a product of interest is exposed to tubing or other similar apparatus, potentially reduces exposure to shear and other fluid forces, and more accurately and flexibly tracks the time spent at low pH. In some embodiments, the residence time is at least 30 minutes. In some embodiments, the residence time is in the range of at least 60 minutes to at least 90 minutes. In some embodiments, the residence time is at least 30 minutes, at least 45 minutes, at least 60 minutes, at least 75 minutes, at least 90 minutes, at least 120 minutes, at least 180 minutes, at least 240 minutes, at least 300 minutes, or at least 360 minutes. In some embodiments, the residence time is at least 30 minutes. In some embodiments, the residence time is at least 45 minutes. In some embodiments, the residence time is at least 60 minutes. In some embodiments, the residence time is at least 75 minutes. In some embodiments, the residence time is at least 90 minutes. In some embodiments, the residence time is at least 120 minutes.

In some embodiments, the residence time from lowering the pH of the affinity chromatography eluate containing the product of interest to collecting the frontal loading product pool containing the product of interest is 1 hour, 2 hours, 3 hours, 4 hours, 5 hours, 6 hours, 7 hours, 8 hours, 9 hours, 10 hours, 12 hours, 16 hours, 18 hours, 20 hours, 24 hours, 30 hours, 36 hours, 42 hours, or 48 hours.

In other embodiments, the pH of the Protein A chromatography effluent may also be adjusted to effectively inactivate viruses while in-line between unit operations instead of in a hold tank. This can be done using any of a variety of known methods, such as in-line mixers, tortuous paths, coiled flow invertors, or other mechanisms that mix and maintain the pH in a fluid flow.

The pH of the Protein A eluate pool or effluent stream is adjusted with any suitable acid at a concentration suitable to meet safety guidelines or regulations for biopharmaceuticals as established by relevant regulatory agencies. In some embodiments, the Protein A eluate pool or effluent stream is adjusted to a pH in the range of 3.0 to 4.0 prior to use as a loading feed for frontal loading chromatography. In some embodiments, the pH is adjusted to a pH in the range of 3.3 to 4.0. In some embodiments, the pH is in the range of 3.3 to 4.0. In some embodiments, the pH is in the range of 3.4 to 4.0. In some embodiments, the pH is in the range of 3.5 to 4.0. In some embodiments, the pH is in the range of 3.6 to 4.0. In some embodiments, the pH is in the range of 3.7 to 4.0. In some embodiments, the pH is in the range of 3.8 to 4.0. In some embodiments, the pH is in the range of 3.9 to 4.0. In some embodiments, the pH is in the range of 3.3 to 3.6. In some embodiments, the pH is in the range of 3.4 to 3.6. In some embodiments, the pH is in the range of 3.5 to 3.6. In some embodiments, the pH is 3.3, 3.4, 3.5, 3.6, 3.7, 3.8, 3.9, or 4.0. In some embodiments, the pH is 3.3. In some embodiments, the pH is 3.6. In some embodiments, the pH is 3.8. In some embodiments, the pH is 4.0.

### Virus Filtration and UF/DF

Non-enveloped viruses are more difficult to inactivate without risk to the protein of interest and are commonly removed by filtration methods. Viral filtration can be performed using micro- or nano-filters, such as those available from PLAVONA® 20 and BioEx (Asahi Kasei, Chicago, IL), VIROSART® CPV, HC, HF, and Media (Sartorius, Goettingen, Germany), VIRESOLVE® Pro and NFP (MilliporeSigma, Burlington, MA), Pegasus™ Prime, VF DV20, DV 50, and SV4 (Pall Biotech, Port Washington, NY), CUNO Zeta Plus VR. (3M, St. Paul, Mn). Viral filtration may occur at one or more steps in the downstream process. Viral filtration may precede or follow an ultrafiltration/diafiltration (UF/DF) operation but may also take place following UF/DF.

Downstream processes typically include at least one ultrafiltration/diafiltration (UF/DF) operation for product concentration and buffer exchange. A UF/DF operation may take place at one or more stages in a downstream process. Typically a UF/DF operation is performed prior to bulk storage of the drug substance to establish the drug substance at a desired concentration and buffer formulation.

In some embodiments, the product pool from the UF/DF operation is directly fed to a fill/finish operation. One or more stability-enhancing excipients may optionally be added directly to the UF/DF retentate feed tank containing the formulated purified protein resulting in formulated drug substance or added to the UF/DF eluate pool prior to fill/finish. An example of a continuous drug substance to drug product operation is provided in WO2020/159838.

Filters for use in a UF/DF operation are well-known and common in the art and are commercially available from many sources. There are many types of UF/DF materials available, including but not limited to, regenerated cellulose Pellicon (MilliporeSigma, Danvers, MA), stabilized cellulose, SARTOCON® Slice, SARTOCON® ECO Hydrosart® (Sartorius, Goettingen, Germany), and polyethersulfone (PES) membrane, Omega (Pall Corporation, Port Washington, NY).

### Proteins

The terms “polypeptide.” “protein,” and “product” are used interchangeably throughout and refer to a molecule comprising two or more amino acid residues joined to each other by peptide bonds. Polypeptides, proteins, and products described herein also include macromolecules having one or more deletions from, insertions to, and/or substitutions of the amino acid residues of a native sequence, that is, a polypeptide, protein, or product produced by a naturally-occurring and non-recombinant cell; or is produced by a genetically-engineered or recombinant cell, and comprise molecules having one or more deletions from, insertions to, and/or substitutions of the amino acid residues of the amino acid sequence of a native protein. The polypeptides, proteins, or products provided herein also include amino acid polymers in which one or more amino acids are chemical analogs of a corresponding naturally-occurring amino acids and polymers. The polypeptides, proteins, or products disclosed herein may also include modifications including, but not limited to, glycosylation, lipid attachment, sulfation, gamma-carboxylation of glutamic acid residues, hydroxylation, and ADP-ribosylation.

The polypeptides, proteins, or products purified according to chromatography methods described herein can be of scientific and/or commercial interest, including protein-based therapeutics. Products of interest include, but are not limited to, secreted proteins, non-secreted proteins, intracellular proteins, or membrane-bound proteins. Polypeptides, proteins, and products of interest can be produced by cell lines (e.g., host cells) using cell culture methods described herein and may be referred to as “recombinant proteins.” The expressed protein(s) may be produced intracellularly or secreted into the culture medium from which it can be recovered and/or collected. The term “isolated protein” or “isolated recombinant protein” refers to a polypeptide or product of interest that is purified away from polypeptides, proteins, or other impurities that would interfere with its therapeutic, diagnostic, prophylactic, research, and/or other use. Products of interest include, but are not limited to, proteins that exert a therapeutic effect by binding a target, such as, e.g., a target among those listed herein, including targets derived therefrom, targets related thereto, and modifications thereof.

Products of interest may include, but are not limited to, “antigen-binding proteins.” An “antigen-binding protein” refers to a protein or polypeptide that comprises an antigen-binding region or antigen-binding portion that has affinity for another molecule to which it binds (e.g., the antigen).

Antigen-binding proteins include, but are not limited to, antibodies, peptibodies, antibody fragments, antibody derivatives, antibody analogs, fusion proteins (including, e.g., single chain variable fragments (scFvs), double-chain (divalent) scFvs, and IgGscFv- (see, e.g., Orcutt et al., 2010, Protein Eng Des Sel 23:221-228)), hetero-IgG (see, e.g., Liu et al., 2015, J Biol Chem 290:7535-7562), muteins, and XMAB® (Xencor, Inc., Monrovia, CA). Also included are bispecific T cell engager molecules (BITE® molecules), bispecific T cell engagers having extensions, chimeric antigen receptors (CARs, CAR Ts), and T cell receptors (TCRs).

In some embodiments, products of interest may include colony stimulating factors, such as granulocyte colony-stimulating factor (G-CSF). Such G-CSF agents include, but are not limited to, NEUPOGEN® (filgrastim) and NEULASTA® (pegfilgrastim).

Also included are erythropoiesis stimulating agents (ESA), such as, EPOGEN® (epoctin alfa), ARANESP® (darbepoetin alfa), DYNEPO® (epoetin delta), MIRCERA® (methyoxy polyethylene glycol-epoetin beta), HEMATIDE®, MRK-2578, INS-22, RETACRIT® (epoctin zeta), NEORECORMON® (epoetin beta), SILAPO® (epoetin zeta), BINOCRIT® (epoetin alfa), epoetin alfa Hexal, ABSEAMED® (epoetin alfa), RATIOEPO® (epoctin theta), EPORATIO® (epoetin theta), BIOPOIN® (epoetin theta), epoetin alfa, epoetin beta, epoetin zeta, epoetin theta, and epoetin delta, epoetin omega, epoetin iota, tissue plasminogen activator, and GLP-1 receptor agonists, as well as variants or analogs thereof and biosimilars of any of the foregoing.

In some embodiments, products of interest bind to one of more of the following, alone or in any combination: CD proteins including, but not limited to, CD3, CD4, CD5, CD7, CD8, CD19, CD20, CD22, CD25, CD30, CD33, CD34, CD38, CD40, CD70, CD123, CD133, CD138, CD171, and CD174, HER receptor family proteins, including, for instance, HER2, HER3, HER4, and the EGF receptor, EGFRvIII, cell adhesion molecules, for example, LFA-1, Mol, p150,95, VLA-4, ICAM-1, VCAM, and alpha v/beta 3 integrin, growth factors, including but not limited to, for example, vascular endothelial growth factor (“VEGF”): VEGFR2, growth hormone, thyroid stimulating hormone, follicle stimulating hormone, luteinizing hormone, growth hormone releasing factor, parathyroid hormone, mullerian-inhibiting substance, human macrophage inflammatory protein (MIP-1-alpha), erythropoietin (EPO), nerve growth factor, such as NGF-beta, platelet-derived growth factor (PDGF), fibroblast growth factors, including, for instance, aFGF and bFGF, epidermal growth factor (EGF), Cripto, transforming growth factors (TGF), including, among others, TGF-α and TGF-β, including TGF-β1, TGF-β2, TGF-β3, TGF-β4, or TGF-β5, insulin-like growth factors-I and -II (IGF-I and IGF-II), des (1-3)-IGF-I (brain IGF-I), and osteoinductive factors, insulins and insulin-related proteins, including, but not limited to, insulin, insulin A-chain, insulin B-chain, proinsulin, and insulin-like growth factor binding proteins; (coagulation and coagulation-related proteins, such as, among others, factor VIII, tissue factor, von Willebrand factor, protein C, alpha-1-antitrypsin, plasminogen activators, such as urokinase and tissue plasminogen activator (“t-PA”), bombazine, thrombin, thrombopoietin, and thrombopoietin receptor, colony stimulating factors (CSFs), including the following, among others, M-CSF, GM-CSF, and G-CSF, other blood and serum proteins, including but not limited to albumin, IgE, and blood group antigens, receptors and receptor-associated proteins, including, for example, flk2/flt3 receptor, obesity (OB) receptor, growth hormone receptors, and T-cell receptors; neurotrophic factors, including but not limited to, bone-derived neurotrophic factor (BDNF) and neurotrophin-3, -4, -5, or -6 (NT-3, NT-4, NT-5, or NT-6); relaxin A-chain, relaxin B-chain, and prorelaxin, interferons, including for example, interferon-alpha, -beta, and -gamma, interleukins (ILs), e.g., IL-1 to IL-10, IL-12, IL-15, IL-17, IL-23, IL-12/IL-23, IL-2Ra, IL1-R1, IL-6 receptor, IL-4 receptor and/or IL-13 to the receptor, IL-13RA2, or IL-17 receptor, IL-1RAP; viral antigens, including but not limited to, an AIDS envelope viral antigen, lipoproteins, calcitonin, glucagon, atrial natriuretic factor, lung surfactant, tumor necrosis factor-alpha and -beta, enkephalinase, BCMA, IgKappa, ROR-1, ERBB2, mesothelin, RANTES (regulated on activation normally T-cell expressed and secreted), mouse gonadotropin-associated peptide, DNase, FR-alpha, inhibin, and activin, integrin, protein A or D, rheumatoid factors, immunotoxins, bone morphogenetic protein (BMP), superoxide dismutase, surface membrane proteins, decay accelerating factor (DAF), AIDS envelope, transport proteins, homing receptors, MIC (MIC-a, MIC-B), ULBP 1-6, EPCAM, addressins, regulatory proteins, immunoadhesins, antigen-binding proteins, somatropin, CTGF, CTLA4, cotaxin-1, MUC1, CEA, c-MET, Claudin-18, GPC-3, EPHA2, FPA, LMP1, MG7, NY-ESO-1, PSCA, ganglioside GD2, ganglioside GM2, BAFF, OPGL (RANKL), myostatin, Dickkopf-1 (DKK-1), Ang2, NGF, IGF-1 receptor, hepatocyte growth factor (HGF), TRAIL-R2, c-Kit, B7RP-1, PSMA, NKG2D-1, programmed cell death protein 1 and ligand, PD1 and PDL1, mannose receptor/hCGβ, hepatitis-C virus, mesothelin dsFv[PE38] conjugate, Legionella pneumophila (Ily), IFN gamma, interferon gamma induced protein 10 (IP10), IFNAR, TALL-1, thymic stromal lymphopoietin (TSLP), proprotein convertase subtilisin/Kexin Type 9 (PCSK9), stem cell factors, Flt-3, calcitonin gene-related peptide (CGRP), OX40L, α4β7, platelet specific (platelet glycoprotein IIb/IIIb (PAC-1), transforming growth factor beta (TFGβ), Zona pellucida sperm-binding protein 3 (ZP-3), TWEAK, platelet derived growth factor receptor alpha (PDGFRα), sclerostin, and biologically active fragments or variants of any of the foregoing.

In some embodiments, products of interest include abciximab, acapatamab, adalimumab, adecatumumab, aflibercept, alemtuzumab, alirocumab, anakinra, atacicept, basiliximab, belimumab, bemarituzumab, bevacizumab, biosozumab, blinatumomab, brentuximab vedotin, brodalumab, cantuzumab mertansine, canakinumab, cetuximab, certolizumab pegol, conatumumab, daclizumab, denosumab, eculizumab, edrecolomab, cfalizumab, cfaveleukin alfa, epratuzumab, erenumab, etanercept, evolocumab, galiximab, ganitumab, gemtuzumab, golimumab, ibritumomab infliximab, ipilimumab, lerdelimumab, letikafusp, lumiliximab, lxdkizumab, mapatumumab, motesanib diphosphate, muromonab-CD3, natalizumab, nesiritide, nimotuzumab, nivolumab, ocrelizumab, ofatumumab, omalizumab, oprelvekin, ordesekimab, palivizumab, panitumumab, pembrolizumab, pertuzumab, pexelizumab, ranibizumab, rilotumumab, rituximab, rocatinlimab, romiplostim, romosozumab, rozibafusp alfa, sargamostim, tarlatamab, tezepelumab, tocilizumab, tositumomab, tiuxetan, trastuzumab, ustekinumab, vedolizumab, visilizumab, volociximab, zanolimumab, and zalutumumab, as well as biosimilars of any of the foregoing.

### EXAMPLES

The following examples are given for the purpose of illustrating various embodiments of the disclosure and are not meant to limit the present disclosure in any fashion. One skilled in the art will readily appreciate that the present disclosure is well-adapted to carry out the objects and obtain the ends and advantages mentioned, as well as those objects, ends, and advantages inherent herein. Changes therein and other uses which are encompassed within the spirit of the disclosure as defined by the scope of the claims will occur to those skilled in the art.

### Example 1. Non-Denaturing Cleaning Methods

Non-limiting design requirements for an effective column cleaning method include robust impurity removal and well-controlled column pressure during cleaning. Impurity removal is important to prevent product carryover and resin fouling in order to prolong resin lifetime and achieve a high number of reuse cycles. Well-controlled column pressure is important for process safety and scalability. Column pressure control during cleaning after frontal chromatography is particularly important because this operating mode leads to a significant amount of bound product and/or bound impurities after the product is loaded, resulting in the column being saturated or near-saturation at typical frontal loadings. Rapid desorption of the bound components can lead to a highly concentrated bolus of product and/or impurities desorbing and migrating through the column, with associated spikes in the pressure drop across the column (Δ pressure=inlet pressure-outlet pressure).

Non-denaturing column cleaning methods were tested for their ability to clean a frontal CEX chromatography column effectively while maintaining stable column pressure. The CEX resin Eshmuno® CP-FT was used for all evaluations, which were conducted according to the following procedures:

### Load Preparation

For each procedure, the load material for frontal CEX chromatography consisted of filtered, neutralized viral inactivated pool (FVIP) containing a monoclonal antibody (mAb1, an IgG2 antibody). The FVIP was prepared by using Protein A chromatography to purify harvest cell culture fluid, with 50 mM sodium acetate used as the Protein A elution buffer. The Protein A pool was subjected to low pH viral inactivation (VI), consisting of titration with 10% acetic acid to pH 3.6±0.1 and neutralization with 2M Tris base to pH 5.0±0.1, unless otherwise noted. The acidified pool was either held for a time duration in the range of 60 minutes to 90 minutes prior to neutralization or immediately neutralized with minimal hold (i.e., mock VI). The VI operation was performed at a temperature in the range of 15° C. to 25° C. VI was followed by depth filtration with a cellulose based-depth filter to generate the final FVIP, i.e., the load for the frontal loading CEX chromatography. The conductivity of the FVIP load varied across these studies, as described below.

### Column Operation

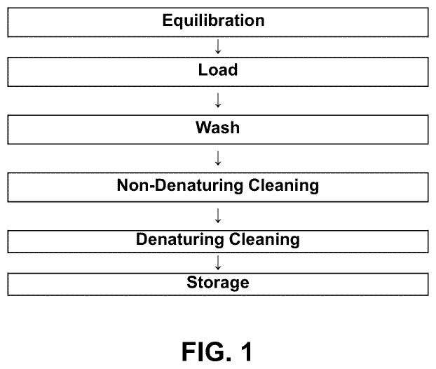

FIG. 1 summarizes the basic operating sequence for a frontal CEX chromatography resin from equilibration to storage. The tested non-denaturing strip phases followed a post-load wash phase and preceded regeneration cleaning with 1 M NaOH and column storage in 0.2 M NaOH. Table 1 provides additional details on the loading and strip cleaning procedures tested. The criteria for the initial evaluation of the strip cleaning procedures consisted of controlling the pressure drop over the column, ΔP, during the strip phase as well achieving near complete cleaning over the prescribed duration of the strip phase.

TABLE 1
Operating Conditions

| | Non-Denaturing | |
|---|---|---|
| Procedure | Cleaning Step | Scale and Operational Details |
| 1 | Isocratic: 10 mM | Bench-scale 1 cm dia × 17 cm |
| | acetate, 2M | bed height column |
| | NaCl, pH 5.5 | Load: FVIP (pH: 4.5, |
| | Duration: <1 CV | conductivity: 4.4 mS/cm) |
| | | Loading: 40 g/L-r |
| | | Product collection: N/A |
| 2 | Isocratic: 50 mM | Bench-scale 1 cm dia × 20 cm |
| | acetate, 1M | bed height column |
| | CaCl₂, pH 5.0 | (cycled 20 times) |
| | Duration: <1 CV | Load: FVIP (pH: 4.6 to 5.2, |
| | | conductivity: 4.3 to |
| | | 7.3 mS/cm) |
| | | Loading: 350 to 900 g/L-r |
| | | Product collection: |
| | | Start collect: 0.5 Optical |
| | | Density (OD) at A280 |
| | | (280 nm absorbance); |
| | | End collect: 1.0 OD at A280 |
| 3 | Linear gradient: | Bench-scale 1 cm dia × 15 cm |
| | 0-100% Buffer B | bed height column |
| | Duration: 10 CVs | (cycled 100 times) |
| | Buffer A: 50 mM | Load: FVIP (pH: 4.6 to 4.9, |
| | acetate, pH 5.0 | conductivity: 6 to 7 mS/cm) |
| | Buffer B: 50 mM | Loading: 300 to 1200 g/L-r |
| | acetate, 500 mM | Product collection: |
| | NaCl, pH 5.0 | Start collect: 0.5 Optical |
| | | Density (OD) at A280 |
| | | (280 nm absorbance); |
| | | End collect: 1.0 OD at A280 |

Procedure 1 utilized an isocratic strip cleaning phase consisting of 10 mM acetate, 2M sodium chloride, pH 5.5. This procedure was screened at bench scale on a 17 cm bed height column with short durations for each phase, including 40 g/L-r column loading and a strip cleaning phase of less than 1 column volume. Due to the low loading for this screening run, a representative product pool was not collected. However, excessive tailing of the UV absorbance was observed during the strip cleaning phase, demonstrating slow desorption of the bound product and impurities even at low loading and suggesting that Procedure 1 may provide inefficient cleaning of the frontal chromatography CEX column.

Procedure 2 employed an isocratic strip cleaning phase with 50 mM acetate, 1M calcium chloride, pH 5.0, and was used during a process robustness study testing various loading conditions over multiple cycles (Table 1). Procedure 2 was moderately effective at cleaning the frontal CEX resin based on the observation of the UV absorbance peak reaching baseline during the strip cleaning phase for individual cycles; however, increasing column pressures were observed over successive cycles of the column when cleaning according to Procedure 2.

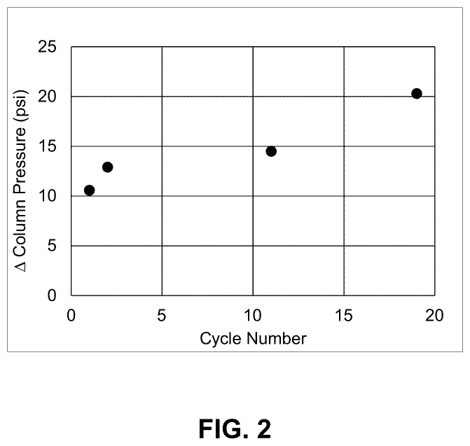

FIG. 2 shows the maximum ΔP observed during the strip cleaning phase for cycles 1, 2, 11, and 19 of the process robustness study. The cycles were performed with 750 g/L-r column loading and load material with pH 5.0 and 5.3 mS/cm conductivity. Although specific limits on operating pressure for any chromatography step vary based on process details such as column hardware, a robust cleaning procedure would be expected to yield minimal change in pressure over successive cycles. As shown in FIG. 2, the maximum ΔP increased by approximately 2-fold over 19 cycles. The observed increase over relatively few cycles at bench scale suggests that cleaning using Procedure 2 may not enable the number of cycles (e.g., 50 or more) typically expected for chromatography resin reuse in large-scale manufacturing, increasing manufacturing costs and limiting its commercial scale utility.

Procedure 3 consisted of a non-denaturing linear salt gradient used to desorb and elute bound product and impurities at a well-controlled rate in order to minimize pressure increases during the cleaning step. Additionally, the high salt concentration achieved at the end of the gradient was designed to enable cleaning over multiple cycles. Sodium chloride was employed to facilitate column regeneration with sodium hydroxide immediately after the strip cleaning phase, a process sequence that may be higher risk if an alternative salt such as calcium chloride was used due to potential precipitation during hydroxide mixing.

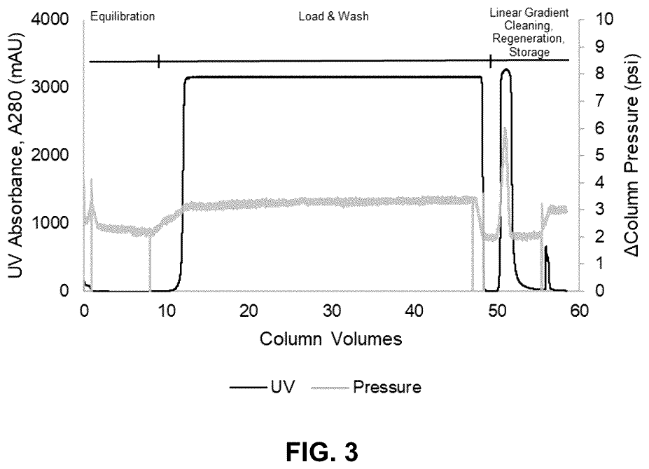

FIG. 3 shows the pressure trend over a representative chromatography run, which demonstrated well-controlled pressure over the strip cleaning. The linear salt gradient of Procedure 3 was also effective at controlling column pressure over multiple cycles, as observed in a resin reuse study that achieved over 100 cycles.

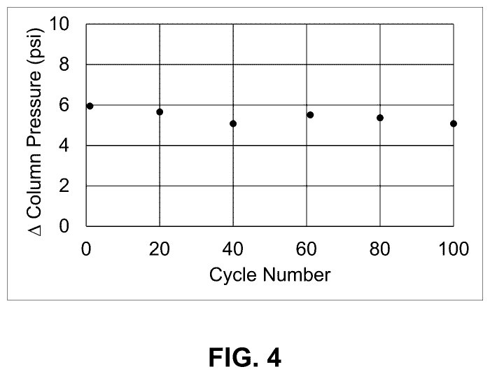

FIG. 4 shows the maximum ΔP observed during the linear gradient strip cleaning phase for representative control runs conducted at 750 g/L-r loading and 150 cm/hr linear velocity for all phases. The relatively constant observed pressures demonstrate the effectiveness of Procedure 3's linear gradient cleaning in minimizing fouling, which tends to increase pressures over successive cycles. An additional objective for column cleaning is to minimize potential product carryover between cycles. This was evaluated for Procedure 3 over 100 cycles by performing a mock frontal CEX run (with a “blank” load consisting of equilibration buffer instead of actual load material), collecting the blank product pool, and testing for trace amounts of protein via a fluorescence-based protein quantification assay (excitation/emission wavelengths: 280/335 nm). The measured protein carryover met the pre-determined acceptance criteria of ≤19.43 μg/mL, which corresponds to 1/1000^{th} of the expected product concentration of the typical product pool, a standard acceptance criterion for this testing.

### Example 2. Impact of Viral Inactivation Acid Titrant on HMW Clearance Over Frontal CEX Column

Pilot-scale studies were performed on a ESHMUNO CP-FT® (EMD Millipore Corporation, Burlington, MA) column operated in frontal mode at high column loading to investigate the impact of the acid titrant used for viral inactivation (VI) on high molecular weight (HMW) clearance over the column. The acid titrant used for viral inactivation (VI) is often selected based on the matrix conditions of the preceding pool; for example, acetic acid is commonly chosen when the Protein A chromatography elution buffer is sodium acetate.

Table 2 summarizes the operating parameters used in the study. For each study condition, a filtered, neutralized viral inactivated pool (FVIP) containing mAb1 was prepared using one of two acid titrants and filtered using a cellulose-based depth filter; the FVIP was used as the load feed for the CEX column. Start and stop collection criteria for the product pool were determined by monitoring the UV absorbance at 280 nm.

Table 3 summarizes the observed HMW clearance for mAb1 pools when 1M formic acid or 10% acetic acid was used as a VI acid titrant. While the absolute HMW % levels in the CEX pool did not vary significantly based on the choice of VI acid titrant, Sample 2 (prepared with formic acid) was loaded at a significantly higher CEX column loading relative to Sample 1 (prepared with acetic acid). Accordingly, the observation of comparable final HMW levels suggests that formic acid use allows for robust impurity clearance over a wider loading range, likely due to the lower FVIP conductivity achieved with formic acid compared to acetic acid. The relative increase in buffer capacity in the acetate-based product pool during VI is lower when using formic acid, which in turn reduces the volume of base required to achieve the target pH during subsequent neutralization and the resulting FVIP conductivity. Reduced load conductivity may lead to stronger electrostatic interactions with the oppositely-charged CEX resin, thereby promoting stronger binding to the resin and greater impurity removal, albeit with somewhat reduced step yield due to greater product retention on the resin.

TABLE 2
Operating Parameters

| | Sample 1 | Sample 2 |
|---|---|---|
| Starting material | mAb1 Protein A pool | mAb1 Protein A pool |
| Protein A elution | 50 mM acetate buffer, pH 3.7 | |
| buffer | | |
| Acid titrant/Base | 10% Acetic | 1M Formic |
| titrant | Acid/2M Tris | Acid/2M Tris |
| Final pH of FVIP | 5.0 | 5.0 |
| Final conductivity | 6.4 | 4.5 |
| (mS/cm) of FVIP | | |
| Chromatography | ESHMUNO CP-FT ® CEX chromatography resin | |
| resin | | |
| Chromatography | 10 cm dia × 15 cm | 20 cm dia × 15 cm |
| column dimensions | bed height (1.2 L) | bed height (4.7 L) |
| Column loading, | 542 | 1100 |
| g/Lr | | |
| Linear velocity | 150 cm/hr | |
| during loading | | |
| Equilibration | 50 mM Acetate; pH 5.0 | |
| Collection start/stop | Start 0.5 AU/cm at A280 | Start 0.5 AU/cm at A280 |
| | Stop 1.0 AU/cm at A280 | Stop 0.75 CVs of wash |
| Wash | 50 mM Acetate; pH 5.0 | 50 mM Acetate; |
| | | 20 mM Sodium |
| | | Chloride; pH 5.0 |

TABLE 3
HMW Removal

| | Sample 1 | Sample 2 |
|---|---|---|
| Starting Material | mAb1 Protein A pool | mAb1 Protein A pool |
| Acid titrant/Base titrant | 10% Acetic | 1M Formic |
| | Acid/2M Tris | Acid/2M Tris |
| Column loading (g/L-r) | 542 | 1100 |
| Step yield (%) | 84.0 | 87.7 |
| % HMW, CEX load | 2.6 | 2.8 |
| (FVIP) | | |
| % HMW, CEX pool | 1.2 | 1.3 |
| Analytical method, | SE-HPLC | SE-HPLC |
| HMW determination | | |

### Example 3. Impact of Viral Inactivation Acid Titrant on Viral Clearance Over Frontal CEX Column

To ensure product safety, a downstream purification process must demonstrate excess retrovirus clearance capability across various steps for virus inactivation (e.g., by low pH) and removal (e.g., by chromatography or filtration). The viral clearance capability of the ESHMUNO CP-FT® resin at bench scale was characterized by spiking FVIP with a model virus, Xenotropic Murine Leukemia virus-related Virus (XMuLV), and loading the spiked FVIP onto the CEX resin at high loading. The FVIP load materials were obtained from pilot-scale runs that utilized either 10% acetic acid or 1M formic acid as the viral inactivation titrant, followed by neutralization to pH 5 and filtration using a cellulose-based depth filter. Table 4 summarizes relevant details of the load preparation and column operation.

Product pool fractions were collected over the course of loading, and virus titers were determined for the load starting material, the various fractions, and a pseudo-pool created to approximate conditions of the product pool. XMuLV titers were determined by an in-house qPCR assay. The virus log reduction value (LRV) over the unit operation was estimated based on the load and the pseudo-pool titers as follows: LRV=log₁₀(Total virus, load/Total virus, pseudo-pool). Table 5 summarizes the results of these studies. For mAb1, the FVIP generated with 1M formic acid led to additional XMuLV clearance of over 1.5 LRVs. The improvement in viral clearance capability with formic acid-generated load may be due to its relatively lower conductivity, which promotes stronger binding of impurities such as adventitious virus.

TABLE 4
Operating Parameters

| | Sample 1 | Sample 2 |
|---|---|---|
| Starting Material | mAb1 Protein A pool | mAb1 Protein A pool |
| Protein A elution | 50 mM acetate buffer, | 50 mM acetate buffer, |
| conditions | pH 3.7 | pH 3.7 |
| Acid titrant/Base | 10% Acetic Acid/ | 1M Formic Acid/ |
| titrant | 2M Tris | 2M Tris |
| Final pH | 5.0 | 5.0 |
| Final Conductivity | 6.4 | 4.5 |
| (mS/cm) of FVIP | | |
| Chromatography | 1 cm dia × 17 cm bed height | |
| column | | |
| dimensions | | |
| Column loading | 1250 | 1100 |
| (g/L-r) | | |
| XMuLV spike | 0.5 | 0.5 |
| percentage | | |
| (% volume) | | |
| Linear velocity | 150 cm/hr | 200 cm/hr |
| during loading | | |
| Equilibration | 50 mM Acetate; | 50 mM Acetate; |
| | 0 mM Sodium | 20 mM Sodium |
| | Chloride; pH 5.0 | Chloride; pH 5.0 |
| Collection | Start 0.5 AU/cm at A280 | Start 0.5 AU/cm at A280 |
| start/stop | Stop 1.0 AU/cm at A280 | Stop 1.0 AU/cm at A280 |

TABLE 5
XMuLV Log Reduction Values

| | Load Conductivity | XMuLV Log Reduction |
|---|---|---|
| | (mS/cm) | Value (LRV) |
| Sample 1 | 6.9 | 2.2 |
| Sample 2 | 4.7 | 3.9 |

### Example 4. CEX Chromatography Yields and Variable Column Volume

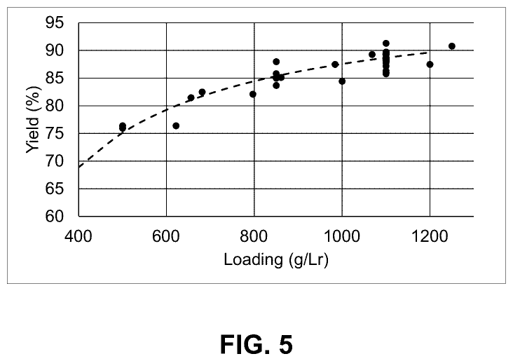

Step yield is an important performance metric for preparative chromatography, particularly for high volume products that require highly productive drug substance processes. Chromatography step yield can vary significantly with column loading for operating modes in which there is significant product binding to the column, e.g., bind and elute and frontal chromatography. In frontal chromatography, yield is expected to increase monotonically with increased loading because of the significant binding of both monomer and impurities to the chromatography resin. As a column is loaded beyond its saturation point and binding sites become occupied, an increasing fraction of the load material flows-through in the absence of free binding sites and is therefore recoverable. This leads to a general trend of increasing step yield with respect to column loading, independent of whether the process includes a post-load wash to recover bound product. FIG. 5 illustrates this trend with pilot-scale and bench-scale process data for frontal CEX chromatography performed on mAb1 pools using the Eshmuno® CP-FT resin. For all depicted runs, the load was FVIP material generated with Protein A pool as the starting material (50 mM acetate buffer, pH 3.7 elution buffer), followed by viral inactivation with 1M formic acid to pH 3.6, neutralization with 2M Tris base to pH 5.0, and depth filtration with a cellulose-based depth filter.

In an idealized large-scale manufacturing scenario, the amount of load mass to be purified over a given sized column leads to a near-optimal column loading (or load factor) with respect to step yield, using multiple column cycles to process the entire batch if necessary. However, the constraints of scaling a process to fit into a given manufacturing site and/or variability in upstream yields often lead to a load mass that does not allow for optimal column loadings. This typically results in either scrapping the excess product that exceeds the maximum allowable loading (thereby decreasing yield), or processing the batch with an additional cycle and using a lower, sub-optimal column loading for each cycle (also decreasing yield due to the process trend mentioned previously). The risk of sub-optimal yields due to upstream mass variability is higher when a given chromatography step requires few cycles to process a batch, as any additional cycle will significantly reduce the loading to process the same amount of material.

Column bed height may be used to modulate column volume in order to achieve a well-controlled and near-optimal loading. In theory, column volume could alternatively be modulated via column diameter; however, this strategy may not be practical in large-scale manufacturing facilities that have limited hardware options. Furthermore, because column volume varies with bed height linearly and with the square of diameter, bed height may provide more granular control of column volume.

In order to modulate column volume based on column bed height, the chromatography process step should be designed to achieve acceptable and consistent performance over a functional bed height range. A bench-scale study was performed to compare the performance of a frontal CEX chromatography step using Eshmuno® CP-FT resin and mAb1 FVIP load material at bed heights of approximately 10 cm and 15 cm. The mAb1 FVIP load material was generated from pilot scale runs, similar to the FVIP loads described above. Table 6 summarizes relevant inputs and a performance indicator (SE-HPLC HMW) used in this study. In addition to study arms in which 10 cm and 15 cm columns were operated at the standard flow rate (“SFR”, i.e. the linear velocity during product loading and wash) for the step, a third arm was included with a 10 cm bed height and the flow rate reduced by 33% (“LFR” condition, i.e. a lower flow rate during product loading and wash) in order to match the residence time of the control arm with 15 cm bed height and 200 cm/hr linear flow rate. Chromatographic resolution typically improves with increasing residence time and bed height; accordingly, the 10 cm, SFR condition were expected to represent the worst case scenario.

Removal of high molecular weight (HMW) species is an important performance indicator for frontal CEX chromatography. SE-HPLC HMW levels were tested for the load (starting levels) as well as for a pseudo-pool (ending levels), which was prepared by combining fractions collected over the course of loading up to 1100 g/L-r. All three conditions achieved HMW % levels of 1.5% or less, exhibiting over a 50% reduction relative to the starting levels. These results indicate that this frontal CEX step for mAb1 may be effectively operated with a range of bed heights, including a bed height of 10 cm, while maintaining a standard process flow rate.

TABLE 6
Bed Height Study Conditions and SE-HPLC Results

| | 10 cm bed | 10 cm bed | |
|---|---|---|---|
| | height, | height | |
| | standard flow | low flow | 15 cm |
| Condition | rate (SFR) | rate (LFR) | bed height |
| Column | 1 cm dia × 10 cm | 1 cm dia × 10 cm | 1 cm dia × 15 cm |
| dimensions | bed height | bed height | bed height |
| Linear velocity | 200 | 133 | 200 |
| during loading & | | | |
| wash (cm/hr) | | | |
| SE-HPLC HMW | 3.3 | 3.3 | 3.3 |
| %, load | | | |
| SE-HPLC HMW | 1.5 | 1.4 | 1.3 |
| %, pseudo-pool | | | |

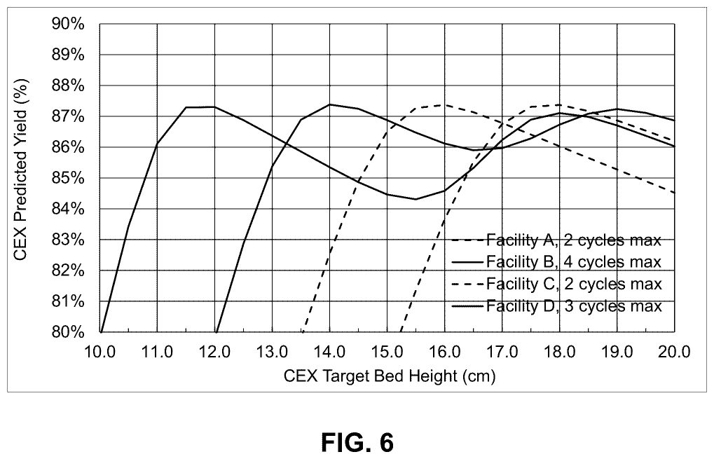

Due to facility constraints on equipment size and forward-processing strategies, it is not uncommon for a given manufacturing facility to present sub-optimal options for column diameter and/or number of chromatography cycles required to process mass within a given time. Designing a process with enhanced flexibility for varying column volume via bed height may limit sub-optimal column loadings due to existing architecture. Process modeling was also performed to predict the impact of varying the column bed height on frontal CEX step yield. FIG. 6 shows results from this modeling, which predicts that on average, the step yield could vary by up to approximately 7% based on bed height for a given set of facility assumptions. As shown, the maximum predicted step yield was approximately 87% (achievable at the maximum loading for the frontal CEX step), and a wide range for the target bed height allows this maximum to be achieved under different manufacturing scenarios.

All documents, or portions of documents, cited in this application, including but not limited to patents, patent applications, articles, books, and treatises, are hereby expressly incorporated by reference. What is described in an embodiment of the disclosure can be combined with one or more other embodiments of the disclosure unless context clearly indicates otherwise.

The disclosed subject matter is not intended to be limited in scope by the specific embodiments described herein, which are instead intended as non-limiting illustrations of individual aspects of the disclosure. Functionally equivalent methods and components are within the scope of the disclosure. Indeed, various modifications of the disclosed subject matter, in addition to those shown and described herein, will be apparent to those skilled in the art from the foregoing description and accompanying drawings. Such modifications are intended to fall within the scope of the disclosed subject matter.

The descriptions of the various embodiments and/or examples of the disclosed subject matter have been presented for purposes of illustration but are not intended to be exhaustive or limiting in any way. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, and/or to enable others of ordinary skill in the art to understand the disclosed subject matter.

What is claimed is:

1. A method of cleaning a chromatography medium for reuse, comprising:
loading a composition comprising a protein and at least one impurity onto the chromatography medium, wherein the loading density exceeds the dynamic binding capacity of the chromatography medium for the protein;
collecting fractions comprising the protein; and
cleaning the chromatography medium using a linear salt gradient.

2. The method of claim 1, wherein the chromatography medium is a cation exchange (CEX) chromatography resin and is packed in a chromatography column.

3. The method of claim 1, wherein the loading density is in the range of 1000 g/L-r to 1500 g/L-r.

4. The method of claim 1, wherein the chromatography medium is loaded at or near saturation with the at least one impurity.

5. The method of claim 1, wherein the linear salt gradient comprises an increase in salt concentration from less than 50 mM to 500 mM.

6. The method of claim 1, wherein the linear salt gradient is an NaCl gradient, a KCl gradient, a CaCl₂ gradient, or an Na₂SO₄ gradient.

7. The method of claim 1, wherein the linear salt gradient is an NaCl gradient.

8. The method of claim 1, wherein the linear salt gradient is generated using at least two buffers, wherein each of the at least two buffers used to generate the linear salt gradient is independently selected from acetate buffers, phosphate buffers, Tris buffers, and 2-(N-morpholino) ethanesulfonic acid buffers.

9. The method of claim 8, wherein each of the at least two buffers used to generate the linear salt gradient comprises acetate.

10. The method of claim 8, wherein the pH of each of the at least two buffers used to generate the linear salt gradient is greater than 3.6 and less than 5.6.

11. The method of claim 1, wherein the linear salt gradient is generated by Buffer A and Buffer B, wherein Buffer A comprises 50 mM acetate and 0 mM or 20 mM sodium chloride at a pH value of 5.0±0.1 and Buffer B comprises 50 mM acetate and 500 mM sodium chloride at a pH value of 5.0±0.1.

12. The method of claim 1, wherein the slope of the linear salt gradient is less than or equal to 0.07 M salt/medium volume buffer.

13. The method of claim 12, wherein the gradient length is at least 7 medium volumes.

14. The method of claim 1, wherein the cleaning using the linear salt gradient is non-denaturing.

15. The method of claim 1, wherein the method enables reuse of the chromatography medium for at least 25 cycles.

16. A method for purifying a protein from a composition comprising the protein and at least one impurity, comprising:
equilibrating a chromatography medium using an equilibration buffer;
loading the composition comprising the protein and the at least one impurity onto the chromatography medium in frontal mode at a loading density of at least 500 g/L-r;
collecting fractions comprising the protein;
passing less than 2 medium volumes of a post-load wash solution through the chromatography medium and adding the eluate to the fractions;
cleaning the chromatography medium using a linear salt gradient; and
cleaning the chromatography medium using a denaturing solution.

17. The method of claim 16, wherein fraction collection begins at 0.5 OD, A280 and is stopped after less than or equal to one medium volume of the post-load wash solution is passed through the chromatography medium.

18. The method of claim 16, wherein the equilibration buffer does not contain salt.

19. The method of claim 16, wherein the denaturing solution is an used in an isocratic cleaning process.

20. The method of claim 16, wherein the chromatography medium is a cation exchange (CEX) chromatography resin and is packed in a chromatography column.

21. A method for improving step yield for a frontal chromatography operation, comprising:
estimating an amount of a protein in a load solution; and
determining a bed height for a frontal chromatography medium based on a target residence time, wherein the flow rate is a constant value in the range of 50 cm/hr to 250 cm/hr.

22. A method for improving step yield for a frontal chromatography operation, comprising:
estimating an amount of a protein in a load solution; and
determining a flow rate for the frontal chromatography operation based on a target residence time, wherein the flow rate is in the range of 50 cm/hr to 250 cm/hr and a bed height for a frontal chromatography medium is a constant value in the range of 5 cm to 30 cm.
